(19) United States
(12) Patent Application Publication
(10) Pub. No.: US 2003/0078369 A1
(43) Pub. Date: Apr. 24, 2003

(54) Apicidin-derived cyclic tetrapeptides

(76) Inventors: Peter T. Meinke, Plainfield, NJ (US); Dennis Schmatz, Cranford, NJ (US); Robert W. Myers, Cresskill, NJ (US); Sandra J. Rattray, Somerset, NJ (US); Steven L. Colletti, Princeton Junction, NJ (US); Matthew J. Wyvratt, Mountainside, NJ (US); Michael H. Fisher, Ringoes, NJ (US); Anne M. Gurnett, New York, NY (US)

Correspondence Address: MERCK AND CO INC, P O BOX 2000, RAHWAY, NJ, 070650907

(21) Appl. No.: 10/066,451

(22) Filed: Jan. 31, 2002

Related U.S. Application Data
(63) Continuation-in-part of application No. 09/614,793, filed on Jul. 12, 2000.
(60) Provisional application No. 60/145,329, filed on Jul. 23, 1999.

(51) Int. Cl. C07K 7/54; C07D 245/00

(52) U.S. Cl. 530/317; 540/460

(57) ABSTRACT

Cyclic tetrapeptide compounds derived from apicidin therapeutically inhibit histone deacetylase activity, are represented by Formula I:

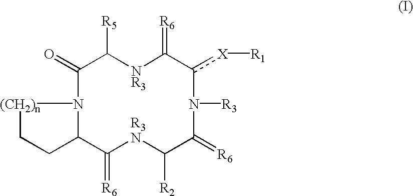

and are useful in the treatment of protozoal infections.

This is a continuation-in-part of U.S. patent application No. 09/614,793, filed Jul. 12, 2000, which claims the benefit of U.S. Patent Application No. 60/145,329, filed Jul. 23, 1999.

### BACKGROUND OF THE INVENTION

1. Field of the invention

The present invention relates to anti-protozoal agents. In particular, the present invention relates to cyclic tetrapeptide compounds derived from apicidin that therapeutically inhibit histone deacetylase activity by protozoa.

2. Related background

Parasitic protozoa are responsible for a wide variety of infections in man and animals. Many of the diseases are life threatening to the host and cause considerable economic loss in animal husbandry. Malaria remains a significant health threat to humans despite massive international attempts to eradicate the disease. Trypanosomiasis such as i) Chagas disease caused by Trypanosome cruzi and ii) African sleeping sickness caused by T. brucei are not uncommon in Africa and South America. Furthermore; opportunistic infections, caused by Pneumocystis carinii, Toxoplasma gondii, and Cryptosporidium sp., in immunocompromised hosts are becoming increasingly significant in developed countries.

A protozoal infection of great economic importance is coccidiosis, a widespread disease of domesticated animals produced by infections by protozoa of the genus Eimeria. Some of the most significant of Eimeria species are those in poultry, namely E. tenella, E. acervulina, E. necatrix, E. praecox, E. mitis, E. brunetti and E. maxima. Coccidiosis can cause high levels of morbidity and mortality in poultry, resulting in extreme economic losses.

In some protozoal diseases, such as Chagas disease, there is no satisfactory treatment. In other protozoal diseases, drug-resistant strains of the protozoa may develop or have developed. Accordingly, there exists a continued need to identify new and effective anti-protozoal drugs. However, antiparasitic drug discovery has been, for the most part, a random and laborious process—through biological screening of natural products and synthetic compounds against a panel of parasites. Drug discovery can be greatly facilitated and made more directed if a specific target of antiprotozoal drugs can be identified, and incorporated into the screening process.

Histone deacetylase (“HDA”) and histone acetyltransferase (“HAT”) together control the net level of acetylation of histones. Inhibition of the action of HDA results in the accumulation of hyperacetylated histones, which in turn is implicated in a variety of cellular responses, including altered gene expression, cell differentiation and cell-cycle arrest. Recently, trichostatin A and trapoxin A have been reported as reversible and irreversible inhibitors, respectively, of mammalian HDA (see e.g., Yoshida et al., BioAssays, 17(5), 423-430 (1995)). Trichostatin A has also been reported to inhibit partially purified yeast HDA (Sanchez del Pino et al., Biochem. J., 303, 723-729 (1994)). Trichostatin A is an antifungal antibiotic and has been shown i) to have anti-trichomonal activity as well as cell differentiating activity in murine erythroleukemia cells, and ii) the ability to induce phenotypic reversion in sis-transformed fibroblast cells (see e.g., U.S. Pat. No. 4,218,478; Yoshida et al., BioAssays, 17(5), 423-430 (1995); and references cited therein). Trapoxin A, a cyclic tetrapeptide, induces morphological reversion of v-sis-transformed NIH3T3 cells (Yoshida and Sugita, Jap. J. Cancer Res., 83(4), 324-328 (1992).

HDA inhibition as a target for cancer research is described in Saito et al., Proc. Natl Acad. Sci. USA, 96, 4592-4597(1999); Bernardi et al., Amino Acids 6, 315-318 (1994); and R. E. Shute et al., J. Med. Chem. 30, 71-78 (1987).

U.S. Pat. No. 5,620,953 describes novel cyclic tetrapeptides, including apicidin. Apicidin [cyclo(N-O-methyl-L-Trp-L-Ile-D-Pip-L-2-amino-8-oxo-decanoyl)] is a broad-spectrum antiprotozoal, antifungal and antineoplastic agent isolated from the fermentation culture of Fusarium fungus. The structure of apicidin is shown below:

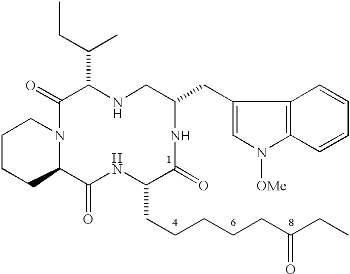

Nevertheless, there remains a need to develop novel antiprotozoic compounds. The present inventors have found that a number of cyclic tetrapeptides derived from apicidin, structurally related to trapoxin A, are inhibitors of histone deacetylase and possess antiprotozoal activity.

### SUMMARY OF THE INVENTION

The present invention relates to novel cyclic tetrapeptides and pharmaceutical compositions containing the tetrapeptides. The invention also concerns a method for treating protozoal infections by administering to a host suffering from protozoal infection a therapeutically effective amount of a compound that inhibits histone deacetylase. Additionally, the invention relates to the use of known cyclic tetrapeptides to inhibit histone deacetylase activity and effective as antiprotozoal agents.

This invention relates i) to new antiprotozoal, antifungal and antineoplastic agents related to apicidin, ii) to processes for preparation of such novel agents, iii) to compositions containing such novel agents, iv) to the use of such novel agents in the treatment of parasitic infections, including malaria, in human and animals and v) the use of such novel agents in treating cancer.

In treating cancer the compounds of this invention can be used as cytostatic compounds, as agents in treating abnormal cell differentiation or proliferation, as agents against tumor growth, or as antimitotic agents for cancer chemotherapy.

### DETAILED DESCRIPTION OF THE INVENTION

In one aspect, according to one embodiment, the present invention relates to a novel cyclic tetrapeptide represented by Formula I shown below:

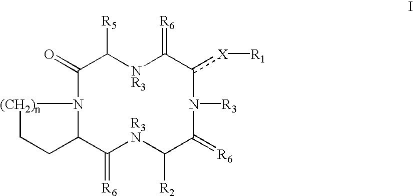

or a pharmaceutically acceptable salt thereof wherein

X is

(1) —CH₂—,

(2) —C(O)—,

(3) —CH(OR^{a})—,

(4) ═CH—, or

(5) not present;

n is

(1) one, or

(2) two;

R₁ is

(1) R⁷,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, ORₐ, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent;

R₂ is

(1) optionally substituted C₂-C₁₂alkyl,

(2) optionally substituted C₂-C₁₂alkenyl,

(3) optionally substituted C₂-C₁₂alkynyl, or

(4) (CH₂)ₙᵢᵢ—O—(CH₂)ₘᵢᵢ wherein nii, mii=0 to 7,

wherein the optional substituents on the alkyl, alkenyl, and alkynyl are 1 to 8 groups and each group independently is

(a) CO₂R^{a},

(b) C(O)R^{b},

(c) C(O)N(OR^{b})R^{c},

(d) C(O)NR^{c}R^{d},

(e) C(O)NR^{c}NR^{c}R^{d},

(f) C(O)NR^{c}SO₂R₇,

(g) C₃-C₈cycloalkyl,

(h) C₂-C₅alkenyl,

(i) cyano,

(j) ═NOR^{a},

(k) ═NNR^{b}R^{c},

(l) ═NNR^{b}S(O)ₙᵢR₇,

(m) N(OR^{b)C(O)NR}^{b}R^{c},

(n) N(OR^{b})C(O)R₇,

(o) NHC(O)N(OR^{b})R^{c},

(p) NR^{c}CO₂R^{b},

(q) NR^{c}C(O)NR^{c}R^{d},

(r) NR^{c}C(S)NR^{c}R^{d},

(s) NR^{c}C(O)R₇,

(t) NR^{b}S(O)ₙᵢR₇,

(u) NR^{c}CH₂CO₂R^{a},

(v) NR^{c}C(S)R₇,

(x) NR^{c}C(O)CH₂OH,

(y) NR^{c}C(O)CH₂SH,

(z) NR^{c}CH₂CO₂R^{a},

(aa) NR^{c}CH₂CH(OH)R₇,

(bb) NR^{c}P(O)(OR^{a})R₇,

(cc) NY¹Y², wherein Y¹ and Y² are independently H or C₁-C₁₀alkyl,

(dd) N₂O,

(ee) N(OR^{b})C(O)R^{b},

(ff) C₁-C₁₀alkanoylamino,

(gg) OR^{a},

(hh) OS(O)ₙᵢR₇,

(ii) oxo,

(jj) OCO₂R^{b},

(kk) OC(O)NR^{c}R^{d},

(ll) P(O)(OR^{a})₂,

(mm) P(O)(OR^{a})R₇,

(nn) SC(O)R₇,

(oo) S(O)ₙᵢR₇,

(pp) SR₇,

(qq) S(O)ₙᵢNR^{c}R^{d},

(rr) NR^{c}CH₂CO₂R^{a},

(ss) diazo,

(tt) C₁-C₅ perfluoroalkyl,

(uu) B(O)(OR^{a})OR^{a},

(vv) halogen,

(ww) aryl(C₀-C₅alkyl), wherein the aryl is optionally substituted with 1 to 3 groups, wherein each group independently is R^{f}, or

(xx) a 3- to 8-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle may be saturated or partly unsaturated;

R₃ each independently is

(1) hydrogen,

(2) halogen,

(3) OR^{a},

(4) C₁-C₄alkyl, or

(5) C₁-C₄aryl;

R₅ is

(1) isopropyl, or

(2) sec-butyl;

R₆ each independently is

(1) O,

(2) S, or

(3) H;

R₇ is (1) hydrogen,

(2) optionally substituted C₂-C₁₀alkyl,

(3) optionally substituted C₂-C₁₀alkenyl,

(4) optionally substituted C₂-C₁₀alkynyl,

(5) optionally substituted C₃-C₈cycloalkyl,

(6) optionally substituted C₅-C₈cycloalkenyl,

(7) optionally substituted aryl,

wherein the optional substituents on the alkyl, alkenyl, alkynyl, cycloalkyl, cycloalkenyl and aryl are 1 to 4 groups, and each group independently is

(a) C₁-C₅alkyl,

(b) X¹-C₁-C₁₀alkyl, wherein X¹ is O or S(O)ₙᵢ,

(c) C₃-C₈cycloalkyl,

(d) hydroxy,

(e) halogen,

(f) cyano,

(g) carboxy,

(h) NY¹Y², wherein Y¹ and Y² are independently H or C₁-C₁₀alkyl,

(i) nitro,

(j) C₁-C₁₀alkanoylamino,

(k) aroyl amino wherein the aroyl is optionally substituted with 1 to 3 groups wherein each group independently is R^{f1}, wherein R^{f1} is defined by any of the definitions below for R^{f} except for (14), (26), (27), and (32),

(l) oxo,

(m) aryl C₀-C₅alkyl wherein the aryl is optionally substituted with 1 to 3 groups, wherein each group independently is R^{f1},

(n) C₁-C₅perfluoroalkyl,

(o) N(OR^{b})C(O)R_{7′}, wherein R₇′ is any of the above definitions of R₇ from (1) to (7)(n), and below of R₇ from (8) to (12), or

(p) NR^{c}C(O)R_{7′},

(8) a 5- to 10-membered heterocycle containing from 1 to 4 heteroatoms, each heteroatom independently is oxygen, sulfur or nitrogen and the heterocycle is optionally substituted by 1 to 3 groups, each group independently is R^{f1}, and the heterocycle may be saturated or partly unsaturated,

(9) a benzene ring fused to a 5- to 10-membered heterocyclic ring containing from 1 to 4 heteroatoms, each heteroatom independently is oxygen, sulfur or nitrogen and the heterocycle is optionally substituted by 1 to 3 groups, each group independently is R^{f1}, and the heterocycle may be saturated or partly unsaturated,

(10) a 5- to 10-membered heterocyclic ring containing from 1 to 4 heteroatoms fused to a second 5- to 10-membered heterocyclic ring containing from 1 to 4 heteroatoms, each heteroatom in either heterocyclic ring independently is oxygen, sulfur or nitrogen and the second heterocyclic ring is optionally substituted by 1 to 3 groups, each group independently is R^{f1}, and each heterocycle independently may be saturated or partly unsaturated,

(11) a benzene ring fused to a C₃-C₈cycloalkyl ring, wherein the cycloalkyl is optionally substituted by 1 to 3 groups each independently being R^{f1}, and the cycloalkyl ring may be saturated or partly unsaturated, or

(12) a 5- to 10-membered heterocyclic ring containing from 1 to 4 heteroatoms, each heteroatom independently is oxygen, sulfur or nitrogen, the heterocyclic ring is fused to a C₃-C₈cycloalkyl ring, wherein the cycloalkyl ring is optionally substituted by 1 to 3 groups each independently being R^{f1}, and the cycloalkyl ring may be saturated or partly unsaturated,

R^{a} is

(1) hydrogen,

(2) optionally substituted C₁-C₁₀alkyl,

(3) optionally substituted C₃-C₁₀alkenyl,

(4) optionally substituted C₃-C₁₀alkynyl,

(5) optionally substituted C₁-C₁₀alkanoyl,

(6) optionally substituted C₃-C₁₀alkenoyl,

(7) optionally substituted C₃-C₁₀alkynoyl,

(8) optionally substituted aroyl,

(9) optionally substituted aryl,

(10) optionally substituted C₃-C₇cycloalkanoyl,

(11) optionally substituted C₅-C₇cycloalkenoyl,

(12) optionally substituted C₁-C₁₀alkylsulfonyl,

(13) optionally substituted C₃-C₈cycloalkyl,

(14) optionally substituted C₅-C₈cycloalkenyl,

wherein the optional substituents on the C₁-C₁₀alkyl, C₃-C₁₀alkenyl, C₃-C₁₀alkynyl, C₁-C₁₀alkanoyl, C₃-C₁₀alkenoyl, C₃-C₁₀alkynoyl, aroyl, aryl, C₃-C₇cycloalkanoyl, C₅-C₇cycloalkenoyl, C₁-C₁₀alkylsulfonyl, C₃-C₈cycloalkyl and C₅-C₈cycloalkenyl are from 1 to 10 groups, wherein each group independently is hydroxy, C₁-C₆alkoxy, C₃-C₇cycloalkyl, aryl C₁l-C₃alkoxy, NR^{x}R^{x}X, CO₂R^{b}, CONR^{c}R^{d}, or halogen,

(15) C₁-C₅perfluoroalkyl,

(16) arylsulfonyl optionally substituted with 1 to 3 groups, wherein each group independently is C₁-C₅alkyl, C₁-C₅perfluoroalkyl, nitro, halogen or cyano,

(17) a 5- or 6-membered heterocycle containing 1 to 4 heteroatoms, wherein each heteroatom is oxygen, sulfur or nitrogen, wherein the heterocycle is optionally substituted by 1 to 4 groups, wherein each group independently is C₁-C₅alkyl, C₁-C₅alkenyl, C₁-C₅perfluoroalkyl, amino, C(O)NR^{c}R^{d}, cyano, CO₂R^{b} or halogen, and wherein the heterocycle may be saturated or partly unsaturated, or

(18) OP(O)(OR^{b})₂;

R^{b} is

(1) H,

(2) optionally substituted aryl,

(3) optionally substituted C₁-C₁₀alkyl,

(4) optionally substituted C₃-C₁₀alkenyl,

(5) optionally substituted C₃-C₁₀alkynyl,

(6) optionally substituted C₃-C₁₅cycloalkyl,

(7) optionally substituted C₅-C₁₀cycloalkenyl, or

(8) optionally substituted 5- to 10-membered heterocycle containing 1 to 4 heteroatoms, wherein each heteroatom independently is oxygen, sulfur, or nitrogen,

wherein the optional substituents on the aryl, C₁-C₁₀alkyl, C₃-C₁₀alkenyl, C₃-C₁₀alkynyl l, C₃-C₁₅cycloalkyl, C₅-C₁₀cycloalkenyl, or 5- to 10-membered heterocycle are from 1 to 10 groups, wherein each group independently is

(a) hydroxy,

(b) C₁-C₆alkyl,

(c) oxo,

(d) SO₂NR^{x}R^{x},

(e) aryl C₁-C₆alkoxy,

(f) hydroxy C₁-C₆alkyl,

(g) C₁-C₁₂alkoxy,

(h) hydroxy C₁-C₆alkoxy,

(I) amino C₁-C₆alkoxy,

(j) cyano,

(k) mercapto,

(l) (C₁-C₆alkyl)—S(O)ₙᵢ—(C₀-C₆alkyl),

(m) C₃-C₇cycloalkyl optionally substituted with 1 to 4 groups, wherein each group independently is R^{e},

(n) C₅-C₇cycloalkenyl,

(o) halogen,

(p) C₁-C₅alkanoyloxy,

(q) C(O)NR^{x}R^{x},

(r) CO₂Rⁱ,

(s) formyl,

(t) —NR^{x}R^{x},

(u) 5 to 9-membered heterocycle, which may be saturated or partially unsaturated, containing from 1 to 4 heteroatoms, wherein each heteroatom independently is oxygen, sulfur or nitrogen, and the heterocycle is optionally substituted with 1 to 5 groups, wherein each group independently is R^{e},

(v) optionally substituted aryl, wherein the optional substituents are 1,2-methylenedioxy or 1 to 5 groups, wherein each group independently is R^{e},

(x) optionally substituted aryl C₁-C₃alkoxy, wherein the optional substituents are 1,2-methylenedioxy or 1 to 5 groups, wherein each group independently is R^{e}, or

(y) C₁-C₅perfluoroalkyl;

R^{c} and R^{d} are independently selected from R^{b}; or R^{c} and R^{d} together with the N to which they are attached form a 3- to 10-membered ring containing 0 to 2 additional heteroatoms, each additional heteroatom independently being oxygen, nitrogen, or (O)ₙᵢ substituted sulfur, wherein the ring is optionally substituted with 1 to 3 groups, wherein each group independently is R^{g}, hydroxy, thioxo, or oxo;

R^{e} is

(1) halogen,

(2) C₁-C₇alkyl,

(3) C₁-C₃perfluoroalkyl,

(4) —S(O)ₘRⁱ,

(5) cyano,

(6) nitro,

(7) RⁱO(CH₂)ᵥ—,

(8) RⁱCO₂(CH₂)ᵥ—,

(9) RⁱOCO(CH₂)ᵥ,

(10) optionally substituted aryl wherein the optional substituents are from 1 to 3 groups, wherein each group independently is halogen, C₁-C₆alkyl, C₁-C₆alkoxy, or hydroxy,

(11) SO₂NR^{x}R^{x},

(12) CO₂R^{x}, or

(13) NR^{x}R^{x};

R^{f} is

(1) C₁-C₄alkyl,

(2) X¹-C₁-C₄alkyl, wherein X¹ is O or S(O)ₘᵢ,

(3) C₂-C₄alkenyl,

(4) C₂-C₄ alkynyl,

(5) C₁-C₃perfluoroalkyl,

(6) NY³Y⁴, wherein Y³ and Y⁴ are each independently hydrogen, C₁-C₅alkyl, or SO₂R^{b},

(7) hydroxy,

(8) halogen,

(9) C₁-C₅alkanoyl amino,

(10) (C₀-C₄alkyl)CO₂R^{a},

(11) (C₀-C₄alkyl)C(O)NR^{b}R^{c},

(12) (C₀-C₄alkyl)NY⁵Y⁶ wherein Y⁵ and Y⁶ together with the N to which they are attached form a 3- to 7-membered ring containing 0 to 2 additional heteroatoms, wherein the additional heteroatoms independently are oxygen, nitrogen, or (O)ₘᵢ substituted sulfur, wherein the ring is optionally substituted with 1 to 3 groups, wherein each group independently is Re or oxo,

(13) (C₀-C₄alkyl)NO₂,

(14) (C₀-C₄alkyl)C(O)R₇,

(15) (C₀-C₄alkyl)CN,

(16) oxo,

(17) (C₀-C₄alkyl)C(O)N(OR^{b})R^{c},

(18) (C₀-C₄alkyl)C(O)NR^{c}R^{d},

(19) (C₀-C₄alkyl)NH^{c}(O)OR^{b},

(20) (C₀-C₄alkyl)NHC(O)NR^{c}R^{d},

(21) (C₀-C₄alkyl)OR^{a},

(22) (C₀-C₄alkyl)OCO₂R^{b},

(23) (C₀-C₄alkyl)OC(O)NR^{c}R^{d},

(24) (C₀-C₄alkyl)C(O)NR^{c}NR^{c}R^{d},

(25) (C₀-C₄alkyl)C(O)NR^{c}SO₂R^{b},

(26) (C₀-C₄alkyl)OS(O)ₙᵢR₇,

(27) (C₀-C₄alkyl)NR^{b}S(O)ₙᵢR₇,

(28) C₀-C₄alkyl halogen,

(29) (C₀-C₄alkyl) SR^{a},

(30) P(O)(OR^{a})₂,

(31) C₀-C₄alkyl azide,

(32) C₀-C₄aryl substituted with from 1 to 4 groups, wherein each group independently is S(O)₂R₇, or

(33) C₀-C₄aryl where the aryl group is optionally substituted from 1 to 4 groups, wherein each group independently is CO₂R^{b}, C(O)NR^{c}R^{d}, NO₂, halogen, OC(O)R^{a}, OR^{a} or C₁-C₄alkyl;

R^{g} and R^{h} together with the N to which they are attached form a 3- to 7-membered ring containing 0 to 2 additional heteroatoms, wherein each additional heteroatom independently is oxygen, nitrogen, or (O)ₘᵢ substituted sulfur, and the ring is optionally substituted with 1 to 3 groups, wherein each group independently is Re or oxo; or

R^{g} and R^{h} are each independently

(1) hydrogen,

(2) C₁-C₆alkyl optionally substituted with hydroxy, amino, or CO₂Rⁱ,

(3) aryl optionally substituted with halogen, 1,2-methylenedioxy, C₁-C₇alkoxy, C₁-C₇alkyl, or C₁-C₃perfluoroalkyl,

(4) aryl C₁-C₆alkyl, wherein the aryl is optionally substituted with C₁-C₃perfluoroalkyl or 1,2-methylenedioxy,

(5) C₁-C₅alkoxycarbonyl,

(6) C₁-C₅alkanoyl,

(7) C₁-C₅alkanoyl C₁-C₆alkyl,

(8) arylC₁-C₅ alkoxycarbonyl,

(9) aminocarbonyl,

(10) (C₁-C₅monoalkyl)aminocarbonyl,

(11) (C₁-C₅dialkyl)aminocarbonyl, or

(12) CO₂R^{b};

Rⁱ is

(1) hydrogen,

(2) C₁-C₃perfluoroalkyl,

(3) C₁-C₆alkyl, or

(4) optionally substituted aryl C₀-C₆alkyl, wherein the aryl optional substituents are from 1 to 3 groups, wherein each group independently is halogen, C₁-C₆alkyl, C₁-C₆alkoxy, or hydroxy;

R^{x} is a C₁-C₄alkyl;

m is 0 to 2;

mi is 0 to 2;

ni is 0 to 2;

mii is 0 to 7;

nii is 0 to 7;

v is 0 to 3; and

excluding apicidin, N-desmethoxy apicidin and compounds represented by the following chemical Formula IIA and Formula IIB:

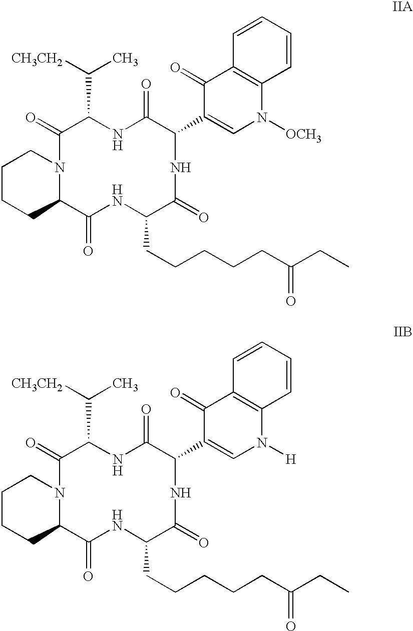

Within this embodiment, the novel cyclic tetrapeptide of this invention includes a genus of compounds represented by Formula I or a pharmaceutically acceptable salt thereof wherein:

X is

(1) —CH₂—,

(2) —C(O)—,

(3) —CH(OR^{a})—,

(4) ═CH—, or

(5) not present; and

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(13) OS(O)ₙᵢR₇,

(14) NR^{b}S(O)ₙᵢR₇, wherein ni is from 0 to 2,

(15) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(16) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(17) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within this genus there is a class of compounds represented by Formula I or a pharmaceutically acceptable salt thereof wherein:

X is

(1) —CH₂—,

(2) —C(O)—,

(3) —CH(OR^{a})—,

(4) ═CH—, or

(5) not present;

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent; and

R₂ is

(1) optionally substituted C₂-C₁₂alkyl,

(2) optionally substituted C₂-C₁₂alkenyl,

(3) optionally substituted C₂-C₁₂alkynyl, or

(4) (CH₂)ₙᵢᵢ—O—(CH₂)ₘᵢᵢ wherein nii, mii=0 to 7,

wherein the optional substituents on the C₂-C₁₂alkyl, C₂-C₁₂alkenyl, and C₂-C₁₂alkynyl are 1 to 8 groups and each group independently is

(a) CO₂R^{a},

(b) C(O)R^{b},

(c) C(O)N(OR^{b})R^{c},

(d) C(O)NR^{c}R^{d},

(e) C(O)NR^{c}NR^{c}R^{d},

(f) C(O)NR^{c}SO₂R₇,

(g) C₃-C₈cycloalkyl,

(h) C₂-C₅alkenyl,

(i) cyano,

(j) ═NOR^{a},

(k) ═NNR^{b}R^{c},

(l) ═NNR^{b}S(O)ₙᵢR₇,

(m) N(OR^{b})C(O)NR^{b}R^{c},

(n) N(OR^{b})C(O)R₇,

(o) NHC(O)N(OR^{b})R^{c},

(p) NR^{c}CO₂R^{b},

(q) NR^{c}C(O)NR^{c}R^{d},

(r) NR^{c}C(S)NR^{c}R^{d},

(s) NR^{c}C(O)R₇,

(t) NR^{b}S(O)ₙᵢR₇,

(u) NR^{c}CH₂CO₂R^{a},

(v) NR^{c}C(S)R₇,

(x) NR^{c}C(O)CH₂OH,

(y) NR^{c}C(O)CH₂SH,

(z) NR^{c}CH₂CO₂R^{a},

(aa) NR^{c}CH₂CH(OH)R₇,

(bb) NR^{c}P(O)(OR^{a})R₇,

(cc) NY¹Y², wherein Y¹ and Y² are independently H or C₁-C₁₀alkyl,

(dd) NO₂,

(ee) N(OR^{b})C(O)R^{b},

(ff) C₁-C₁₀alkanoyl amino,

(gg) OR^{a},

(hh) OS(O)ₙᵢR₇,

(ii) oxo,

(jj) OCO₂R^{b},

(kk) OC(O)NR^{c}R^{d},

(ll) P(O)(OR^{a})₂,

(mm) P(O)(OR^{a})R₇,

(nn) SC(O)R₇,

(oo) S(O)ₙᵢR₇,

(pp) SR₇,

(qq) S(O)ₙᵢNR^{c}R^{d},

(rr) NR^{c}CH₂CO₂R^{a},

(ss) diazo,

(tt) C₁-C₅ perfluoroalkyl,

(uu) B(O)(OR^{a})OR^{a},

(vv) halogen,

(ww) aryl(C₀-C₅alkyl), wherein the aryl is optionally substituted with 1 to 3 groups, wherein each group independently is R^{f}, or

(xx) a 3- to 8-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle may be saturated or partly unsaturated.

Within the above class of compounds, there is a subclass of compounds represented by Formula I or a pharmaceutically acceptable salt thereof wherein n is 1 or 2.

Within this genus there is another class of compounds represented by Formula I or a pharmaceutically acceptable salt thereof wherein:

X is

(1) —CH₂—,

(2) —C(O)—, or

(3) not present; and

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within the above class of compounds, there is a subclass of compounds represented by Formula I or a pharmaceutically acceptable salt thereof wherein n is 1 or 2.

Within this genus there is yet another class of compounds represented by Formula I or a pharmaceutically acceptable salt thereof wherein:

X is

(1) —CH₂—,

(2) —C(O)—, or

(3) not present; and

R₁ is

(1) R₇,

(2) C(O)R₇,

(3) CO₂R^{b},

(4) C(O)N(OR^{b})R^{c},

(5) C(O)NR^{c}R^{d},

(6) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(7) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(8) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within the above class of compounds, there is a subclass of compounds represented by Formula I or a pharmaceutically acceptable salt thereof wherein n is 1 or 2.

Within this genus there is yet another class of compounds represented by Formula I or a pharmaceutically acceptable salt thereof wherein:

X is

(1) —CH₂—,

(2) —C(O)—, or

(3) not present;

R₁ is

(1) R₇,

(2) C(O)R₇,

(3) CO₂R^{b},

(4) C(O)N(OR^{b})R^{c},

(5) C(O)NR^{c}R^{d},

(6) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(7) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(8) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent; and

R₂ is

(1) optionally substituted C₂-C₁₂alkyl,

(2) optionally substituted C₂-C₁₂alkenyl,

(3) optionally substituted C₂-C₁₂alkynyl, or

(4) (CH₂)ₙᵢᵢ—O—(CH₂)ₘᵢᵢ wherein nii, mii=0 to 7,

wherein the optional substituents on the C₂-C₁₂alkyl, C₂-C₁₂alkenyl, and C₂-C₁₂alkynyl are 1 to 5 groups and each group independently is

(a) CO₂R^{a},

(b) C(O)R^{b},

(c) C(O)N(OR^{b})R^{c},

(d) C(O)NR^{c}R^{d},

(e) C(O)NR^{c}NR^{c}R^{d},

(f) C(O)NR^{c}SO₂R₇,

(g) C₃-C₈cycloalkyl,

(h) C₂-C₅alkenyl,

(i) cyano,

(j) ═NOR^{a},

(k) ═NNR^{b}R^{c},

(l) ═NNR^{b}S(O)ₙᵢR₇,

(m) N(OR^{b})C(O)NR^{b}R^{c},

(n) N(OR^{b})C(O)R₇,

(o) NHC(O)N(OR^{b})R^{c},

(p) NR^{c}CO₂R^{b},

(q) NR^{c}C(O)NR^{c}R^{d},

(r) NR^{c}C(S)NR^{c}R^{d},

(s) NR^{c}C(O)R₇,

(t) NR^{b}S(O)ₙᵢR₇,

(u) NR^{c}CH₂CO₂R^{a},

(v) NR^{c}C(S)R₇,

(x) NR^{c}C(O)CH₂OH,

(y) NR^{c}C(O)CH₂SH,

(z) NR^{c}CH₂CO₂R^{a},

(aa) NR^{c}CH₂CH(OH)R₇,

(bb) NR^{c}P(O)(OR^{a})R₇,

(cc) NY¹Y², wherein Y¹ and Y² are independently H or methyl,

(dd) NO₂,

(ee) N(OR^{b})C(O)R^{b},

(ff) C₁-C₃alkanoylamino,

(gg) OR^{a},

(hh) OS(O)ₙᵢR₇,

(ii) oxo,

(jj) OCO₂R^{b},

(kk) OC(O)NR^{c}R^{d},

(ll) P(O)(OR^{a})₂,

(mm) P(O)(OR^{a})R₇,

(nn) SC(O)R₇,

(oo) S(O)ₙᵢR₇,

(pp) SR₇,

(qq) S(O)ₙᵢNR^{c}R^{d},

(rr) NR^{c}CH₂CO₂R^{a},

(ss) diazo,

(tt) C₁-C₅ perfluoroalkyl,

(uu) B(O)(OR^{a})OR^{a},

(vv) halogen,

(ww) aryl(C₀-C₅alkyl), wherein the aryl is optionally substituted with 1 to 3 groups, wherein each group independently is R^{f}, or

(xx) a 3- to 6-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle may be saturated or partly unsaturated.

Within the above class of compounds, there is a subclass of compounds represented by Formula I or a pharmaceutically acceptable salt thereof wherein n is 1 or 2.

Within this embodiment there is a second genus of compounds represented by Formula I or a pharmaceutically acceptable salt thereof wherein:

R₃ each independently is

(1) hydrogen,

(2) halogen,

(3) OR^{a},

(4) C₁-C₄alkyl, or

(5) C₁-C₄aryl; and

Ra is

(1) hydrogen,

(2) optionally substituted C₁-C₆alkyl,

(3) optionally substituted C₃-C₆alkenyl,

(4) optionally substituted C₂-C₄alkanoyl,

(5) optionally substituted C₃-C₄alkenoyl,

(6) optionally substituted aroyl,

(7) optionally substituted aryl,

(8) optionally substituted C₅-C₆cycloalkanoyl,

(9) optionally substituted C₁-C₄alkylsulfonyl,

(10) optionally substituted C₅-C₆cycloalkyl,

(11) optionally substituted C₅-C₆cycloalkenyl, wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,

(12) CF₃,

(13) arylsulfonyl optionally substituted with 1 to 3 groups, wherein each group independently is methyl, CF₃, nitro, halogen or cyano, or

(14) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle may be saturated or partly unsaturated.

Within this second genus is a class of compounds represented by Formula I or a pharmaceutically acceptable salt thereof wherein:

R₃ each independently is

(1) hydrogen,

(2) halogen,

(3) OR^{a},

(4) C₁-C₄alkyl, or

(5) C₁-C₄aryl;

R^{a} is

(1) hydrogen,

(2) optionally substituted C₁-C₆alkyl,

(6) optionally substituted C₃-C₆alkenyl,

(7) optionally substituted C₂-C₄alkanoyl,

(5) optionally substituted C₃-C₄alkenoyl,

(6) optionally substituted aroyl,

(7) optionally substituted aryl,

(8) optionally substituted C₅-C₆cycloalkanoyl,

(9) optionally substituted C₁-C₄alkylsulfonyl,

(10) optionally substituted C₅-C₆cycloalkyl,

(11) optionally substituted C₅-C₆cycloalkenyl,

wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,

(12) CF₃,

(13) arylsulfonyl optionally substituted with 1 to 3 groups, wherein each group independently is methyl, CF₃, nitro, halogen or cyano, or

(14) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle may be saturated or partly unsaturated.

X is

(1) —CH₂—,

(2) —C(O)—,

(3) ═CH—, or

(4) not present; and

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within the above class of compounds, there is a subclass of compounds represented by Formula I or a pharmaceutically acceptable salt thereof wherein n is 1 or 2.

Within this second genus is a class of compounds represented by Formula I or a pharmaceutically acceptable salt thereof wherein:

R3 each independently is

(1) hydrogen,

(2) halogen,

(3) OR^{a},

(4) C₁-C₄alkyl, or (5) C₁-C₄aryl;);

R^{a} is

(1) hydrogen,

(2) optionally substituted C₁-C₆alkyl,

(3) optionally substituted C₃-C₆alkenyl,

(4) optionally substituted C₂-C₄alkanoyl,

(5) optionally substituted C₃-C₄alkenoyl,

(6) optionally substituted aroyl,

(7) optionally substituted aryl,

(8) optionally substituted C₅-C₆cycloalkanoyl,

(9) optionally substituted C₁-C₄alkylsulfonyl, (10) optionally substituted C₅-C₆cycloalkyl, (11) optionally substituted C₅-C₆cycloalkenyl, wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,

(12) CF₃,

(13) arylsulfonyl optionally substituted with 1 to 3 groups, wherein each group independently is methyl, CF₃, nitro, halogen or cyano, or

(14) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle may be saturated or partly unsaturated;

X is

(1) —CH₂—,

(2) —C(O)—,

(3) ═CH—, or

(4) not present; and

R₁ is

(1) R₇,

(2) C(O)R₇,

(9) CO₂R^{b},

(10) C(O)N(OR^{b})R^{c},

(11) C(O)NR^{c}R^{d},

(12) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(13) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(14) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within the above class of compounds, there is a subclass of compounds represented by Formula I or a pharmaceutically acceptable salt thereof wherein n is 1 or 2.

Within this embodiment there is a third genus of compounds represented by Formula I or a pharmaceutically acceptable salt thereof wherein:

R6 each independently is

(1) O,

(2) S, or

(3) H;

X is

(1) —CH₂—,

(2) —C(O)—,

(3) ═CH—, or

(4) not present; and

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₆perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within this third genus is a class of compounds represented by Formula I or a pharmaceutically acceptable salt thereof wherein n is 1 or 2.

Within this third genus is a class of compounds represented by Formula I or a pharmaceutically acceptable salt thereof wherein:

R₃ each independently is

(1) hydrogen,

(2) halogen,

(3) OR^{a},

(4) C₁-C₄alkyl, or

(5) C₁-C₄aryl;

R₆ each independently is

(1) O,

(2) S, or

(3) H;

X is

(1) —CH₂—,

(2) —C(O)—,

(3) ═CH—, or

(4) not present; and

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(O)ₙᵢR₇,

(15) NR^{b}S(O)ₙᵢR₇, wherein ni is from 0 to 2,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

Within the above class of compounds, there is a subclass of compounds represented by Formula I or a pharmaceutically acceptable salt thereof wherein n is 1 or 2.

In an aspect, the invention is directed to a compound represented by Formula I:

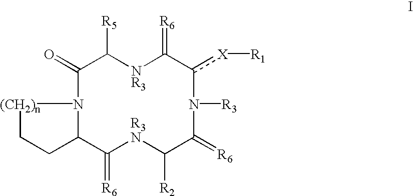

or a pharmaceutically acceptable salt thereof, wherein

X is

(1) —CH₂—,

(2) —C(O)—,

(3) —CH(OR^{a})—,

(4) ═CH—, or

(5) not present;

n is

(1) one, or

(2) two;

R₁ is

(1) R₇,

(2) C(O)R₇,

(3) CN,

(4) CO₂R^{b},

(5) C(O)N(OR^{b})R^{c},

(6) C(O)NR^{c}R^{d},

(7) NHCO₂R^{b},

(8) NHC(O)NR^{c}R^{d},

(9) (C₀-C₄alkyl)OR^{a},

(10) (C₀-C₄alkyl)OCO₂R^{b},

(11) (C₀-C₄alkyl)OC(O)NR^{c}R^{d},

(12) C(O)NR^{c}NR^{c}R^{d},

(13) C(O)NR^{c}SO₂R^{b},

(14) OS(Q)ₙᵢR₇,

(15) NR^{b}S(O)ₙᵢR₇,

(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a}, C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,

(17) a benzene ring fused to a 4- to 8-membered heterocyclic ring with from 1 to 4 heteroatoms, optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or

(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent;

R₂ is

(1) optionally substituted C₂-C₁₂alkyl,

(2) optionally substituted C₂-C₁₂alkenyl,

(3) optionally substituted C₂-C₁₂alkynyl, or

(4) (CH₂)ₙᵢᵢ—O—(CH₂)ₘᵢᵢ—CH₃,

wherein the optional substituents on the C₂-C₁₂alkyl, C₂-C₁₂alkenyl, and C₂-C₁₂alkynyl are 1 to 8 groups and each group independently is

(a) CO₂R^{a},

(b) C(O)R^{b},

(c) C(O)N(OR^{b})R^{c},

(d) C(O)NR^{c}R^{d},

(e) C(O)NR^{c}NR^{c}R^{d},

(f) C(O)NR^{c}SO₂R₇,

(g) C₃-C₈cycloalkyl,

(h) C₂-C₅alkenyl,

(i) cyano,

(j) ═NOR^{a},

(k) ═NNR^{b}R^{c},

(l) ═NNR^{b}S(O)ₙᵢR₇,

(m) N(OR^{b})C(O)NR^{b}R^{c},

(n) N(OR^{b})C(O)R₇,

(o) NHC(O)N(OR^{b})R^{c},

(p) NR^{c}CO₂R^{b},

(q) NR^{c}C(O)NR^{c}R^{d},

(r) NR^{c}C(S)NR^{c}R^{d},

(s) NR^{c}C(O)R₇,

(t) NR^{b}S(O)ₙᵢR₇,

(u) NR^{c}CH₂CO₂R^{a},

(v) NR^{c}C(S)R₇,

(x) NR^{c}C(O)CH₂OH,

(y) NR^{c}C(O)CH₂SH,

(z) NR^{c}CH₂CH(OH)R₇,

(aa) NR^{c}P(O)(OR^{a})R₇,

(bb) NY¹Y², wherein Y¹ and Y² are independently H or C₁-C₁₀alkyl,

(cc) NO₂,

(dd) N(OR^{b})C(O)R^{b},

(ee) C₁-C₁₀alkanoylamino,

(ff) OR^{a},

(gg) OS(O)ₙᵢR₇,

(hh) oxo,

(ii) OCO₂R^{b},

(jj) OC(O)NR^{c}R^{d},

(kk) P(O)(OR^{a})₂,

(ll) P(O)(OR^{a})R₇,

(mm) SC(O)R₇,

(nn) S(O)ₙᵢR₇,

(oo) SR₇,

(pp) S(O)ₙᵢNR^{c}R^{d},

(qq) diazo,

(rr) C₁-C₅ perfluoroalkyl,

(ss) B(O)(OR^{a})OR^{a},

(tt) halogen,

(uu) aryl(C₀-C₅alkyl), wherein the aryl is optionally substituted with 1 to 3 groups, wherein each group independently is R^{f}, or

(vv) a 3- to 8-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle is saturated or partly unsaturated;

R₃ each independently is

(1) hydrogen,

(2) halogen,

(3) OR^{a},

(4) C₁-C₄alkyl, or

(5) aryl;

R₅ is

(1) isopropyl, or

(2) sec-butyl;

R₆ each independently is

(1) O,

(2) S, or

(3) H;

R₇ is

(1) hydrogen,

(2) optionally substituted C₂-C₁₀alkyl,

(3) optionally substituted C₂-C₁₀alkenyl,

(4) optionally substituted C₂-C₁₀alkynyl,

(5) optionally substituted C₃-C₈cycloalkyl,

(6) optionally substituted C₅-C₈cycloalkenyl,

(7) optionally substituted aryl,

wherein the optional substituents on the C₂-C₁₀alkyl, C₂-C₁₀alkenyl, C₂-C₁₀alkynyl, C₃-C₈cycloalkyl, C₅-C₈cycloalkenyl and aryl are 1 to 4 groups, and each group independently is

(a) C₁-C₅alkyl,

(b) X¹-C₁-C₁₀alkyl, wherein X¹ is 0 or S(O)ₙᵢ,

(c) C₃-C₈cycloalkyl,

(d) hydroxy,

(e) halogen,

(f) cyano,

(g) carboxy,

(h) NY¹Y², wherein Y¹ and Y² are independently H or C₁-C₁₀alkyl,

(i) nitro,

(j) C₁-C₁₀alkanoylamino,

(k) aroyl amino wherein the aroyl is optionally substituted with 1 to 3 groups wherein each group independently is Rf¹, wherein R^{f1} is defined by any of the definitions below for R^{f} except for (14), (26), (27), and (32),

(l) oxo,

(m) aryl C₀-C₅alkyl wherein the aryl is optionally substituted with 1 to 3 groups, wherein each group independently is R^{f1},

(q) C₁-C₅perfluoroalkyl,

(r) N(OR^{b})C(O)R_{7′}, wherein R₇′ is any of the above definitions of R₇ from (1) to (7)(m), and below of R₇ from (8) to (12), or

(s) NR^{c}C(O)R_{7′},

(8) a 5- to 10-membered heterocycle containing from 1 to 4 heteroatoms, each heteroatom independently is oxygen, sulfur or nitrogen and the heterocycle is optionally substituted by 1 to 3 groups, each group independently is R^{f1}, and the heterocycle is saturated or partly unsaturated,

(9) a benzene ring fused to a 5- to 10-membered heterocyclic ring containing from 1 to 4 heteroatoms, each heteroatom independently is oxygen, sulfur or nitrogen and the heterocycle is optionally substituted by 1 to 3 groups, each group independently is R^{f1}, and the heterocycle is saturated or partly unsaturated,

(10) a 5- to 10-membered heterocyclic ring containing from 1 to 4 heteroatoms fused to a second 5- to 10-membered heterocyclic ring containing from 1 to 4 heteroatoms, each heteroatom in either heterocyclic ring independently is oxygen, sulfur or nitrogen and the second heterocyclic ring is optionally substituted by 1 to 3 groups, each group independently is R^{f1}, and each heterocycle independently is saturated or partly unsaturated,

(11) a benzene ring fused to a C₃-C₈cycloalkyl ring, wherein the cycloalkyl is optionally substituted by 1 to 3 groups each independently being R^{f1}, and the cycloalkyl ring is saturated or partly unsaturated, or

(12) a 5- to 10-membered heterocyclic ring containing from 1 to 4 heteroatoms, each heteroatom independently is oxygen, sulfur or nitrogen, the heterocyclic ring is fused to a C₃-C₈cycloalkyl ring, wherein the cycloalkyl ring is optionally substituted by 1 to 3 groups each independently being R^{f1}, and the cycloalkyl ring is saturated or partly unsaturated,

R^{a} is

(1) hydrogen,

(2) optionally substituted C₁-C₁₀alkyl,

(3) optionally substituted C₃-C₁₀alkenyl,

(4) optionally substituted C₃-C₁₀alkynyl,

(5) optionally substituted C₁-C₁₀alkanoyl,

(6) optionally substituted C₃-C₁₀alkenoyl,

(7) optionally substituted C₃-C₁₀alkynoyl,

(8) optionally substituted aroyl,

(9) optionally substituted aryl,

(10) optionally substituted C₃-C₇cycloalkanoyl,

(11) optionally substituted C₅-C₇cycloalkenoyl,

(12) optionally substituted C₁-C₁₀alkylsulfonyl,

(13) optionally substituted C₃-C₈cycloalkyl,

(14) optionally substituted C₅-C₈cycloalkenyl, wherein the optional substituents on the C₁-C₁₀alkyl, C₃-C₁₀alkenyl, C₃-C₁₀alkynyl, C₁-C₁₀alkanoyl, C₃-C₁₀alkenoyl, C₃-C₁₀alkynoyl, aroyl, aryl, C₃-C₇cycloalkanoyl, C₅-C₇cycloalkenoyl, C₁-C₁₀alkylsulfonyl, C₃-C₈cycloalkyl and C₅-C₈cycloalkenyl are from 1 to 10 groups, wherein each group independently is hydroxy, C₁-C₆alkoxy, C₃-C₇cycloalkyl, aryl C₁-C₃alkoxy, NR^{x}R^{x}, CO₂R^{b}, CONR^{c}R^{d}, or halogen,

(15) C₁-C₅perfluoroalkyl,

(16) arylsulfonyl optionally substituted with 1 to 3 groups, wherein each group independently is C₁-C₅alkyl, C₁-C₅perfluoroalkyl, nitro, halogen or cyano,

(17) a 5- or 6-membered heterocycle containing 1 to 4 heteroatoms, wherein each heteroatom is oxygen, sulfur or nitrogen, wherein the heterocycle is optionally substituted by 1 to 4 groups, wherein each group independently is C₁-C₅alkyl, C₂-C₅alkenyl, C₁-C₅perfluoroalkyl, amino, NMe₂, C(O)NR^{c}R^{d}, cyano, CO₂R^{b} or halogen, and wherein the heterocycle is saturated or partly unsaturated, or

(18) OP(O)(OR^{b})₂;

R^{b} is

(1) H,

(2) optionally substituted aryl,

(3) optionally substituted C₁-C₁₀alkyl,

(4) optionally substituted C₃-C₁₀alkenyl,

(5) optionally substituted C₃-C₁₀alkynyl,

(6) optionally substituted C₃-C₁₅cycloalkyl,

(7) optionally substituted C₅-C₁₀cycloalkenyl, or

(8) optionally substituted 5- to 10-membered heterocycle containing 1 to 4 heteroatoms, wherein each heteroatom independently is oxygen, sulfur, or nitrogen,

wherein the optional substituents on the aryl, C₁-C₁₀alkyl, C₃-C₁₀alkenyl, C₃-C₁₀alkynyl, C₃-C₁₅cycloalkyl, C₅-C₁₀cycloalkenyl, or 5- to 10-membered heterocycle are from 1 to 10 groups, wherein each group independently is

(a) hydroxy,

(b) C₁-C₆alkyl,

(c) oxo,

(d) SO₂NR^{x}R^{x},

(e) aryl C₁-C₆alkoxy,

(f) hydroxy C₁-C₆alkyl,

(g) C₁-C₁₂alkoxy,

(h) hydroxy C₁-C₆alkoxy,

(i) amino C₁-C₆alkoxy,

(j) cyano,

(k) mercapto,

(l) (C₁-C₆alkyl)—S(O)ₙᵢ—(C-C₆alkyl),

(m) C₃-C₇cycloalkyl optionally substituted with 1 to 4 groups, wherein each group independently is R^{e},

(n) C₅-C₇cycloalkenyl,

(o) halogen,

(p) C₁-C₅alkanoyloxy, p2 (q) C(O)NR^{x}R^{x},

(r) CO₂Rⁱ,

(s) formyl,

(t) NR^{x}R^{x},

(u) 5 to 9-membered heterocycle, which is saturated or partially unsaturated, containing from 1 to 4 heteroatoms, wherein each heteroatom independently is oxygen, sulfur or nitrogen, and the heterocycle is optionally substituted with 1 to 5 groups, wherein each group independently is R^{e},

(v) optionally substituted aryl, wherein the optional substituents are 1,2-methylenedioxy or 1 to 5 groups, wherein each group independently is R^{e},

(w) optionally substituted aryl C₁-C₃alkoxy, wherein the optional substituents are 1,2-methylenedioxy or 1 to 5 groups, wherein each group independently is R^{e}, or

(x) C₁-C₅perfluoroalkyl;

R^{c} and R^{d} are independently selected from R^{b}; or R^{c} and R^{d} together with the N to which they are attached form a 3- to 10-membered ring containing 0 to 2 additional heteroatoms, each additional heteroatom independently being oxygen, nitrogen, or (O)ₙᵢ substituted sulfur, wherein the ring is optionally substituted with 1 to 3 groups, wherein each group independently is R^{g}, hydroxy, thioxo, or oxo;

R^{e} is

(1) halogen,

(2) C₁-C₇alkyl,

(3) C₁-C₃perfluoroalkyl,

(4) —S(O)ₘRⁱ,

(5) cyano,

(6) nitro,

(7) RⁱO(CH₂)ᵥ—,

(8) RⁱCO₂(CH₂)ᵥ—,

(9) RⁱOCO(CH₂)ᵥ,

(10) optionally substituted aryl wherein the optional substituents are from 1 to 3 groups, wherein each group independently is halogen, C₁-C₆alkyl, C₁-C₆alkoxy, or hydroxy,

(11) SO₂NR^{x}R^{x},

(12) CO₂R^{x}, or

(13) NR^{x}R^{x};

R^{f} is

(1) C₁-C₄alkyl,

(2) X¹-C₁-C₄alkyl, wherein X¹ is 0 or S(O)ₘᵢ,

(3) C₂-C₄alkenyl,

(4) C₂-C₄ alkynyl,

(5) C₁-C₃perfluoroalkyl,

(6) NY³Y⁴, wherein Y³ and Y⁴ are each independently hydrogen, C₁-C₅alkyl, or SO₂R^{b},

(7) hydroxy,

(8) halogen,

(9) C₁-C₅alkanoyl amino,

(10) (C₀-C₄alkyl)CO₂R^{a},

(11) (C₀-C₄alkyl)C(O)NR^{b}R^{c},

(12) (C₀-C₄alkyl)NY⁵Y⁶ wherein Y⁵ and Y⁶ together with the N to which they are attached form a 3- to 7-membered ring containing 0 to 2 additional heteroatoms, wherein the additional heteroatoms independently are oxygen, nitrogen, or (O)ₘᵢ substituted sulfur, wherein the ring is optionally substituted with 1 to 3 groups, wherein each group independently is Re or oxo,

(13) (C₀-C₄alkyl)NO₂,

(14) (C₀-C₄alkyl)C(O)R₇,

(15) (C₀-C₄alkyl)CN,

(16) oxo,

(17) (C₀-C₄alkyl)C(O)N(OR^{b})R^{c},

(18) (C₀-C₄alkyl)C(O)NR^{c}R^{d},

(19) (C₀-C₄alkyl)NHC(O)OR^{b},

(20) (C₀-C₄alkyl)NHC(O)NR^{c}R^{d},

(21) (C₀-C₄alkyl)OR^{a},

(22) (C₀-C₄alkyl)OCO₂R^{b},

(23) (C₀-C₄alkyl)OC(O)NR^{c}R^{d},

(24) (C₀-C₄alkyl)C(O)NR^{c}NR^{c}R^{d},

(25) (C₀-C₄alkyl)C(O)NR^{c}SO₂R^{b},

(26) (C₀-C₄alkyl)OS(O)ₙᵢR₇,

(27) (C₀-C₄alkyl)NR^{b}S(O)ₙᵢR₇,

(28) C₀-C₄alkyl halogen,

(29) (C₀-C₄alkyl) SR^{a},

(30) P(O)(OR^{a})₂,

(31) C₀-C₄alkyl azide,

(32) aryl substituted with from 1 to 4 groups, wherein each group independently is S(O)₂R₇, CO₂R^{b}, C(O)NR^{c}R^{d}, NO₂, halogen, OC(O)R^{a}, OR^{a} or C₁-C₄alkyl;

R_{g} is

(1) hydrogen,

(2) C₁-C₆alkyl optionally substituted with hydroxy, amino, or CO₂Rⁱ,

(3) aryl optionally substituted with halogen, 1,2-methylenedioxy, C₁-C₇alkoxy, C₁-C₇alkyl, or C₁-C₃perfluoroalkyl,

(4) aryl C₁-C₆alkyl, wherein the aryl is optionally substituted with C₁-C₃perfluoroalkyl or 1,2-methylenedioxy,

(5) C₁-C₅alkoxycarbonyl,

(6) C₁-C₅alkanoyl,

(7) C₁-C₅alkanoyl C₁-C₆alkyl,

(8) arylC₁-C₅ alkoxycarbonyl,

(9) aminocarbonyl,

(10) (C₁-C₅monoalkyl)aminocarbonyl,

(11) (C₁-C₅dialkyl)aminocarbonyl, or

(12) CO₂R^{b};

Rⁱ is

(1) hydrogen,

(2) C₁-C₃perfluoroalkyl,

(3) C₁-C₆alkyl, or

(4) optionally substituted aryl C₀-C₆alkyl, wherein the aryl optional substituents are from 1 to 3 groups, wherein each group independently is halogen, C₁-C₆alkyl, C₁-C₆alkoxy, or hydroxy;

R^{x} is a C₁-C₄alkyl;

m is 0 to 2;

mi is 0 to 2;

ni is 0 to 2;

mii is 0 to 6;

nii is 0 to 7;

v is 0 to 3; and

excluding apicidin, N-desmethoxy apicidin, chlamydocin, Cly-2, HC-Toxin, Trapoxin A, β-hydroxy-HC-toxin and compounds represented by chemical Formula IIA and chemical Formula IIB:

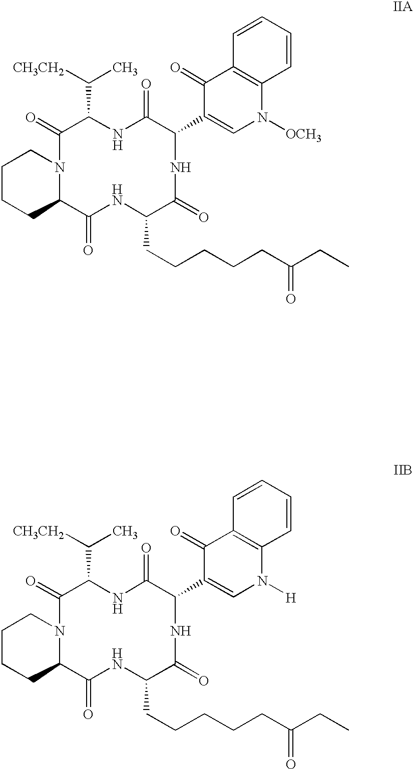

and excluding compounds having the formula IIC

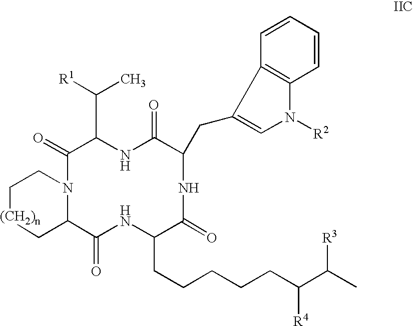

wherein R¹ is CH₃ or CH₂CH₃;

R² is H or —OCH₃;

R³ is H and R⁴ is ═O or (H, OH); or

R³ is OH and R⁴ is ═O or (H, OH); and

n is 0 or 1.

In one aspect, the present invention provides a novel cyclic tetrapeptide represented by Formula I, wherein X is preferably —CH₂—.

In another aspect, the present invention provides a novel cyclic tetrapeptide represented by Formula I, wherein X is preferably —C(O)—.

In still another aspect, the present invention provides a novel cyclic tetrapeptide represented by Formula I, wherein X is preferably not present.

In yet another aspect, the present invention provides a novel cyclic tetrapeptide represented by Formula I, wherein R₁ is preferably a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group may be saturated, partly unsaturated or fully unsaturated, wherein the heteroatoms are each independently oxygen, sulfur, or nitrogen, in which the nitrogen optionally has an R^{c} substituent.

In another aspect, the present invention provides a novel cyclic tetrapeptide represented by Formula I, wherein R₁ is preferably a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group may be saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide.

In another aspect, the present invention provides a novel cyclic tetrapeptide represented by Formula I, wherein R₁ is preferably a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle may be saturated, partly unsaturated or fully unsaturated, and wherein each heteroatom independently is oxygen, sulfur, or nitrogen, and the nitrogen optionally has an R^{c} substituent.

In another aspect, the present invention provides a novel cyclic tetrapeptide represented by Formula I, wherein the compound is Example 2, 3a, 3b, 3d, 10, 11, 12d, 12e, 17, or 18:

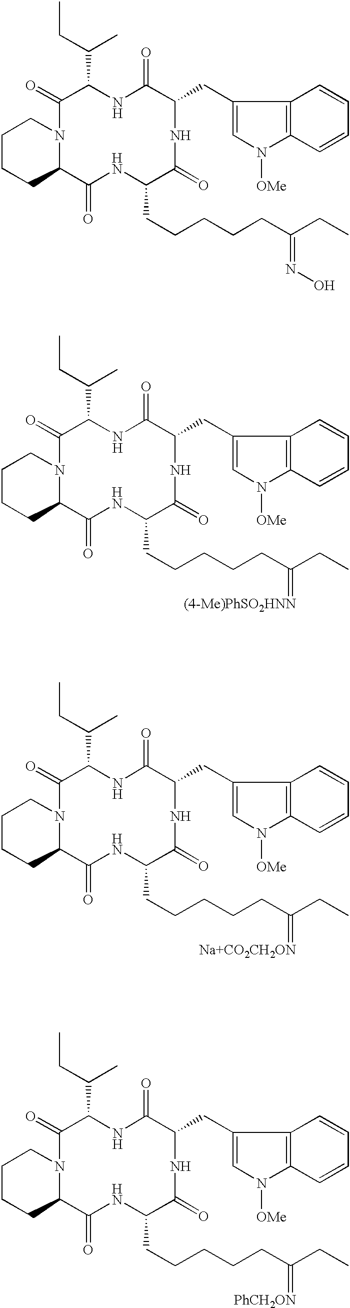

In another aspect, the present invention provides a novel cyclic tetrapeptide represented by Formula I, wherein the compound is Example 22a, 22b, 23a, 23b, 145, 146c, 146d, 146e, 146f, or 147:

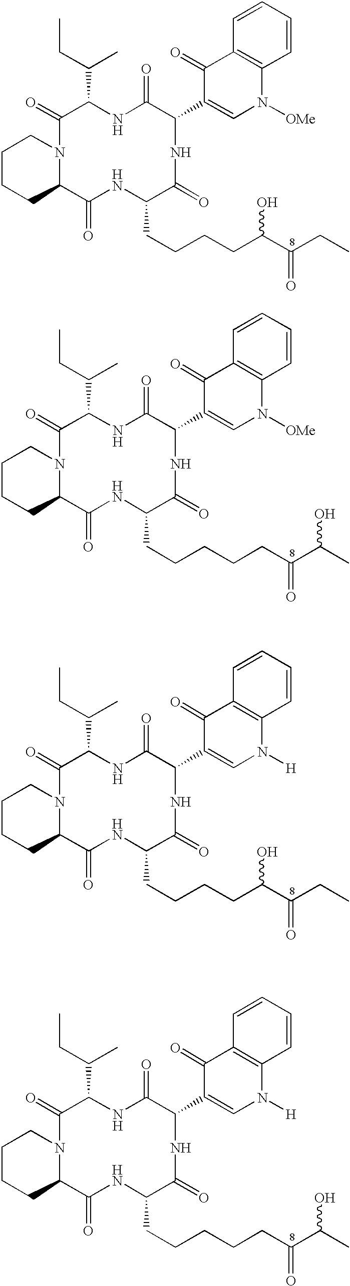

In yet another aspect, the present invention provides a novel cyclic tetrapeptide represented by Formula I, wherein the compound is Example 21a, 21b, 24a, 24b, 26, 27, 28, 29, 30, 32, 37, 39, 43, 44, 46, 51, 56a, 63, 64, or 67:

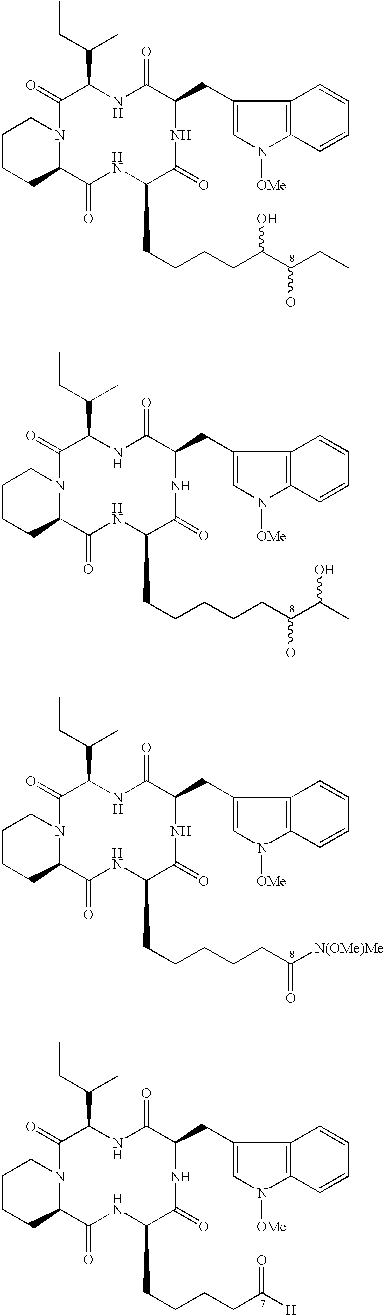

In another aspect, the present invention provides a novel cyclic tetrapeptide represented by Formula I, wherein the compound is Example 69, 70, 72, 73, 74a. 74b, 74c, 74d, 74e, 74f, 74g, 74h, 74i, 74j, 75, 79, 91, 93, 97, 98, 129a, or 129b:

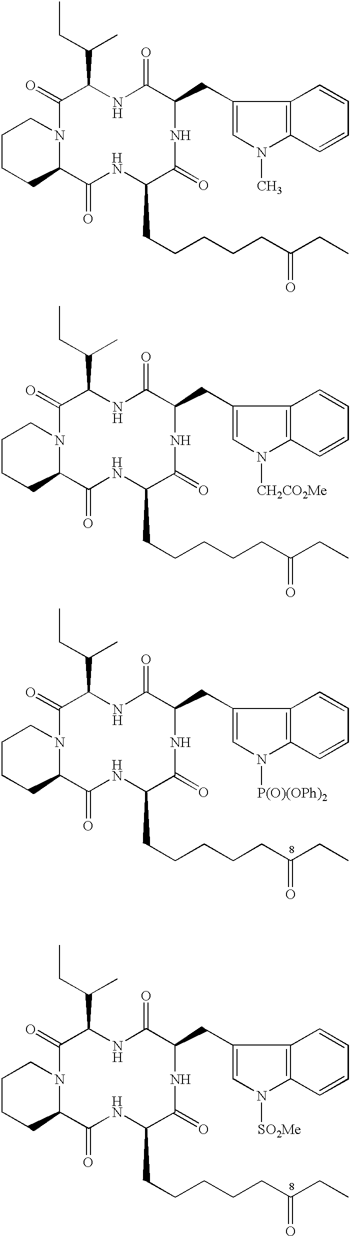

In yet another aspect, the present invention provides a novel cyclic tetrapeptide represented by Formula I, wherein the compound is Example 132a, 133, 135, 138, 139a, 139b, 139c, 139d, 139e, 139f, 139g, 139h, 139i, 139j, 140, 141, 142, 144b, 144d, 144f, 158, 159, 160, 162a, or 162b.

In still another aspect, the present invention provides a novel cyclic tetrapeptide represented by Formula I, wherein the compound is Example 102, 103, 108a, or 108b.

In another aspect, the present invention provides a novel cyclic tetrapeptide represented by Formula I, wherein the compound is Example 109 or 110.

In another aspect, the present invention provides a novel cyclic tetrapeptide represented by Formula I, wherein the compound is Example 168.

In another aspect, the present invention provides a novel cyclic tetrapeptide represented by Formula I, wherein the compound is Example 156, 157a, 157b, 157c, or 157d.

In another aspect, the present invention provides a novel cyclic tetrapeptide represented by Formula I, wherein the compound is

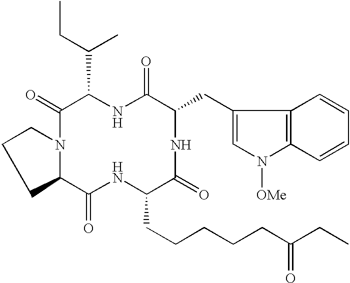

In another aspect, the present invention provides a novel cyclic tetrapeptide represented by Formula I, wherein the compound is

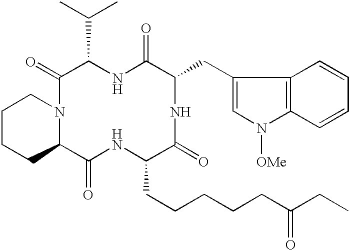

In another aspect, the present invention provides a novel cyclic tetrapeptide represented by Formula I, wherein the compound is Example 153 or 154.

In another aspect, the present invention provides a method for the treatment of protozoal infections comprising the step of administering to a host suffering from a protozoal infection a therapeutically effective amount of the novel compounds of the invention which inhibits histone deacetylase. A therapeutically effective amount is that safe amount sufficient to inhibit histone deacetylase activity of the causative protozoa to control and overcome the infection. The present invention also provides a method for the prevention of protozoal infections comprising the step of administering to a host an effective preventative amount of the novel compounds of the invention, which inhibits histone deacetylase. An effective preventative amount is that safe amount sufficient to inhibit the infection of the host.

In yet another aspect, the present invention provides a composition useful for the treatment or prevention of protozoal diseases which comprises an inert carrier and an effective amount of a compound of formula I.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalene, adamantane, indanyl, indenyl, fluorenyl, 1,2,3,4-tetrahydronaphalene and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “halogen” includes fluorine, chlorine, bromine and iodine atoms.

The term “heterocycle”, unless otherwise specified, means cyclic systems such as those described above for cycloalkyl and cycloalkenyl in which at least one atom is a sulfur, oxygen or nitrogen atom in a group of atoms that form the backbone of a ring. Such heterocycles include mono- or bicyclic compounds that are saturated or partly unsaturated, as well as benzo- or heteroaromatic ring fused saturated heterocycles or partly unsaturated heterocycles, and containing from 1 to 4 heteroatoms independently selected from oxygen, sulfur and nitrogen. Examples of saturated heterocycles include morpholine, thiomorpholine, piperidine, piperazine, tetrahydropyran, tetrahydrofuran, dioxane, tetrahydrothiophene, oxazolidine, pyrrolidine; examples of partly unsaturated heterocycles include dihydropyran, dihydropyridazine, dihydrofuran, dihydrooxazole, dihydropyrazole, dihydropyridine, dihydropyridazine and the like. Examples of benzo- or heteroaromatic ring fused heterocycle include 2,3-dihydrobenzofuranyl, benzopyranyl, tetrahydroquinoline, tetrahydroisoquinoline, benzomorpholinyl, 1,4-benzodioxanyl, 2,3-dihydrofuro(2,3-b)pyridyl and the like.

The term “aryl” is intended to include mono- and bicyclic aromatic and heteroaromatic rings containing from 0 to 5 heteroatoms independently selected from nitrogen, oxygen and sulfur. The term “aryl” is also meant to include benzofused cycloalkyl, benzofused cycloalkenyl, and benzofused heterocyclic groups. Examples of “aryl” groups include phenyl, pyrrolyl, isoxazolyl, pyrazinyl, pyridinyl, oxazolyl, thiazolyl, imidazolyl, triazolyl, tetrazolyl, furanyl, triazinyl, thienyl, pyrimidinyl, pyridazinyl, pyrazinyl, naphthyl, benzoxazolyl, benzothiazolyl, benzimidazolyl, benzofuranyl, furo(2,3-B)pyridyl, 2,3-dihydrofuro(2,3-b)pyridyl, benzoxazinyl, benzothiophenyl, quinolinyl, indolyl, 2,3-dihydrobenzofuranyl, benzopyranyl, 1,4-benzodioxanyl, indanyl, indenyl, fluorenyl, 1,2,3,4-tetrahydronaphthalene and the like.

Aroyl means arylcarbonyl in which aryl is as defined above.

Examples of NR^{c}R^{d} or NR^{g}R^{h} forming a 3- to 10-membered ring containing 0 to 2 additional heteroatoms selected from O, S(O)ₘ and N are aziridine, azetidine, pyrrolidine, piperidine, thiomorpholine, morpholine, piperazine, octahydroindole, tetrahydroisoquinoline and the like.

The term “C₀” means that the carbon is not present. Thus, “C₀-C₅” means that there are from none to five carbons present—that is, five, four, three, two, one, or no carbons present.

The term “optionally substituted” is intended to include both substituted and unsubstituted. Thus, for example, optionally substituted aryl could represent a pentafluorophenyl or a phenyl ring.

Compounds described herein contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I. Further, mixtures of stereoisomers as well as isolated specific stereoisomers are also included. During the the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The concept of the inhibition of histone deacetylase as a target for antiprotozoal compounds is described in pending U.S. patent application 09/296,834, filed Apr. 22, 1999, and U.S. Pat. No. 6,110,697, issued Aug. 29, 2000. Known compounds that may be histone deacetylase inhibitors and therefore useful in the treatment of protozoal diseases include, for example, trichostatin A, trapoxin A and B, HC-toxin, chlamydocin, Cly-2, WF-3161, Tan-1746, apicidin, and analogs thereof. Trapoxin A is described in Itazaki et al., J. Antibiot. 43, 1524-1532(1990); HC-Toxin is described in Liesch et al., Tetrahedron 38, 45-48(1982); chlamydocin is described in Closse et al., Helv. Chim. Acta 57, 533-545(1974); Cly-2 is described in Hirota et al., Agri. Biol. Chem 37, 955-56(1973); WF-3161 is described in Umehana et al., J. Antibiot. 36, 478-483(1983); and Tan-1746 is described in Japanese Patent No. 7196686. Unlike the ethyl ketone sidechain found in apicidin, HC toxin, chlamydocin, trapoxin A and trapoxin B contain a C8 α-ketoepoxide functionality.

Apicidin and analogs thereof referred to herein are described by the following chemical formula:

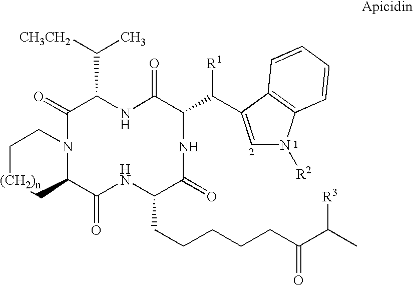

Examples include

| Compound | n | R¹ | R² | R³ |
|---|---|---|---|---|
| Apicidin Ia | 1 | H | OMe | H |
| Ib | 0 | H | OMe | H |
| Ic | 1 | H | OMe | OH |
| IIA | 1 | ═O | OMe | H |
| IIB | 1 | ═O | H | H |

These compounds are described in U.S. patent application No. 08/281,325, filed Jul. 27, 1994, now abandoned, and U.S. Pat. No. 5,620,953, issued Apr. 1, 1997. The compounds are produced from a strain of Fusarium as disclosed in the applications.

The compounds of the present invention have been found to be histone deacetylase inhibitors. Accordingly, they can be useful in the treatment and prevention of protozoal diseases in human and animals, including poultry. Examples of protozoal diseases against which histone deacetylase inhibitors may be used, and their respective causative pathogens, include: 1) amoebiasis (Dientamoeba sp., Entamoeba histolytica); 2) giardiasis (Giardia lamblia); 3) malaria (Plasmodium species including P. vivax, P. falciparum, P. malariae and P. ovale); 4) leishmaniasis (Leishmania species including L. donovani, L. tropica, L. mexicana, and L. braziliensis); 5) trypanosomiasis and Chagas disease (Trypanosoma species including T. brucei, T. theileri, T. rhodesiense, T. gambiense, T. evansi, T. equiperdum, T. equinum, T. congolense, T. vivax and T. cruzi); 6) toxoplasmosis (Toxoplasma gondii); 7) neosporosis (Neospora caninum); 8) babesiosis (Babesia sp.); 9) cryptosporidiosis (Cryptosporidium sp.); 10) dysentary (Balantidium coli); 11) vaginitis (Trichomonas species including T. vaginitis, and T. foetus); 12) coccidiosis (Eimeria species including E. tenella, E. necatrix, E. acervulina, E. maxima and E. brunetti, E. mitis, E. bovis, E. melagramatis, and Isospora sp.); 13) enterohepatitis (Histomonas gallinarum); and 14) infections caused by Anaplasma sp., Besnoitia sp., Leucocytozoan sp., Microsporidia sp., Sarcocystis sp., Theileria sp., and Pneumocystis cannii.

The histone deacetylase inhibiting compounds and compositions of the present invention are preferably used in the treatment or prevention of protozoal infections caused by a member of the sub-phylum Apicomplexans. More preferably the compounds and compositions are used i) in the treatment or prevention of malaria, toxoplasmosis, cryptosporidiosis and trypanosomiasis in humans and animals, and ii) in the management of coccidiosis, particularly in poultry, either to treat coccidial infection or to prevent the occurrence of such infection.

When the histone deacetylase inhibiting compounds and compositions of this invention are administered on a chronic basis, such as in the prevention of coccidiosis in poultry, the histone deacetylase inhibitor preferably is selective for protozoal histone deacetylase over the host histone deacetylase. Such a selective inhibitor would minimize adverse histone deacetylase inhibition effects to the host over the long term.

Two specific examples of the method of this invention of administering an effective preventative amount of an histone deacetylase inhibitor to prevent the establishment of parasitic infections in humans and animals are 1) the prevention of Plasmodium (malaria) infection in humans in endemic areas and 2) the prevention of coccidiosis in poultry. The histone deacetylase-inhibiting compound can be conveniently administered continually in the feed or drinking water, or regularly by oral or parenteral dosing.

Malaria is the number one cause of death in the world. The disease is transmitted by mosquitoes in endemic areas and can very rapidly progress to a life threatening infection. Therefore, individuals living in or visiting areas where malaria carrying mosquitoes are present routinely take prophylactic drugs to prevent infection. Thus, according to an embodiment of the present invention, a histone deacetylase inhibitor is administered orally or parenterally one or more time(s) a day, preferably each dose ranges from about 0.01 mg/kg to about 100 mg/kg. The compound may be administered for the entire period during which the patient or animal is at risk of acquiring a parasitic infection.

Coccidiosis is a disease that can occur in humans and animals and is caused by several genera of coccidia. The most economically important occurrence of coccidiosis is the disease in poultry. Coccidiosis in poultry is caused by protozoan parasites of the genus Eimeria. The disease can spread quite rapidly throughout flocks of birds via contaminated feces. The parasites destroy gut tissue and damage the gut lining, thereby impairing nutrient absorption. An outbreak of coccidiosis in a poultry house can cause such dramatic economic losses for poultry producers that it has become standard practice to use anticoccidial agents prophylactically in the poultry feed. Thus, according to another embodiment of this invention, a histone deacetylase inhibitor is administered in the feed or drinking water for the entire or a portion of the lifetime of domestic birds with a dose that ranges between about 0.1 ppm to about 500 ppm in the feed or water.

For treatment of established parasitic infections in humans or animals, the histone deacetylase inhibitor is conveniently administered orally or parenterally when the infection is suspected or diagnosed. The treatment period varies according to the specific parasitic disease and the severity of the infection. In general the treatment is continued until the parasites are effectively eradicated and/or the symptoms of the disease are resolved. Two specific examples of the method of this invention for the treatment of protozoal infections by administering a therapeutically effective amount of a histone deacetylase inhibitor are 1) the treatment of a Cryptosporidium parvum infection in an animal or human and 2) the treatment of acute Plasmodium falciparum malaria in humans.

Cryptosporidium parvum is a protozoan parasite that infects and destroys cells lining the intestinal tract of humans and animals. The infection establishes quite rapidly and has acute effects on the patient. In the case of humans, patients get severe dysentery for a period of 5-7 days. In immune compromised patients C. parvum infections can persist and can be life threatening. In animals C. parvum infection is the leading cause of death in young dairy calves. A C. parvum infection can be easily diagnosed by symptoms and examination of a stool sample. When the disease is suspected and/or diagnosed, treatment with a histone deacetylase inhibitor according to the method of this invention can be initiated. The dose preferably ranges from about 0.01 mg/kg to about 500 mg/kg. The histone deacetylase is administered one or more time(s) a day, orally or parenterally until the infection is eliminated. The dosing period typically is in the range of about 1-3 weeks.

P. falciparum causes acute life threatening malarial infections in humans. The infection if left untreated can often result in the death of the patient. A malaria infection can be easily diagnosed by symptoms and examination of a blood sample from the patient. Treatment would be initiated following diagnosis. According to an embodiment of this invention, a histone deacetylase inhibitor is administered one or more time(s) a day, orally or parenterally, until the infection is eliminated. The dose preferably ranges from about 0.01 mg/kg to about 200 mg/kg.

The histone deacetylase inhibiting compositions of this invention may be administered to a host in need of treatment in a manner similar to that used for other known antiprotozoal agents. For example, the compositions may be administered parenterally, orally, topically, or rectally. The dosage to be administered will vary according to the particular compound used, the infectious organism involved, the particular host, the severity of the disease, the physical condition of the host, and the selected route of administration; the appropriate dosage can be readily determined by a person skilled in the art. For the treatment of protozoal diseases in human and animals, the dosage preferably ranges from about 0.01 mg/kg to about 500 mg/kg. For prophylactic use in human and animals, the dosage preferably ranges from about 0.01 mg/kg to about 100 mg/kg. For use as an anticoccidial agent, particularly in poultry, the compound is preferably administered in the animals′ feed or drinking water. The dosage preferably ranges from about 0.1 ppm to about 500 ppm.

In one aspect, the composition of the present invention comprises a histone deacetylase inhibitor and an inert carrier. The compositions include pharmaceutical compositions for human and veterinary usage, and feed compositions for the control of coccidiosis in poultry.

The pharmaceutical compositions of the present invention comprise a histone deacetylase inhibitor as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the histone deacetylase inhibitor of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the histone deacetylase inhibitors may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 1 mg to about 500 mg of the active ingredient and each cachet or capsule preferably containing from about 1 to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing the histone deacetylase inhibiting compounds of this invention, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient.

As described above, to manage coccidiosis in poultry, the histone deacetylase inhibitor of this invention can be conveniently administered as a component of a feed composition. The poultry feed preferably contains from about 1 ppm to about 1000 ppm, more preferably from about 10 ppm to about 150 ppm of the histone deacetylase inhibitor of this invention. The optimum levels will vary with the species of Eimeria involved, and can be readily determined by one skilled in the art. It is preferred that the histone deacetylase inhibitor of this invention be added to poultry feed in the amount of from about 0.01% to about 0.1% by weight of the diet. The compositions of this invention are especially useful in controlling the pathology associated with E. tenella. The preferred concentration for similar control of intestinal-dwelling species is from about 0.01% to about 0.1% by weight of the diet. Amounts of about 0.01% to about 0.1% percent by weight are advantageous in reducing the pathogenic effects of both fecal coccidiosis and intestinal coccidiosis.

In the preparation of poultry feed incorporating the compositions of the invention, the histone deacetylase inhibitor can be conveniently dispersed, for example, by i) being mechanically mixed in a finely ground form with the poultry feedstuff, or ii) being first mixed with an intermediate formulation (to form a premix) that is subsequently blended with other poultry feedstuff components. Typical components of poultry feedstuffs include molasses, fermentation residues, corn meal, ground and rolled oats, wheat shorts and middlings, alfalfa, clover and meat scraps, together with mineral supplements such as bone meal, calcium carbonate and vitamins.

Compositions containing a compound described by formula I may also be prepared in powder or liquid concentrate form. In accordance with standard veterinary formulation practice, conventional water-soluble excipients, such as lactose or sucrose, may be incorporated in the powders to improve their physical properties. It is preferable that the powder compositions of this invention comprise from about 50 wt % to about 100 wt %, and more preferably about 60 wt % to about 80 wt % of the compound. These powders may either be added to animal feedstuffs, for example, by way of an intermediate premix, or added to the animal drinking water by dilution.

Liquid concentrates of this invention suitably contain a water-soluble compound combination and may optionally further include a veterinary acceptable water miscible solvent. For example, a solvent such as polyethylene glycol, propylene glycol, glycerol, or glycerol formal can be mixed with up to 30% v/v of ethanol. It is preferable that the liquid concentrates of this invention comprise from about 50 wt % to about 100 wt %, and more preferably about 60 wt % to about 80 wt % of the compound. The liquid concentrates may be administered to the drinking water of animals, particularly poultry.

The following examples are provided to more fully illustrate the present invention, and are not to be construed as limiting the scope of the claims in any manner.

Preparation of Side Chain-Modified Apicidin Analogs

Referring to Scheme I below, apicidin can be converted into alpha-substituted analog compounds 4 and 5.

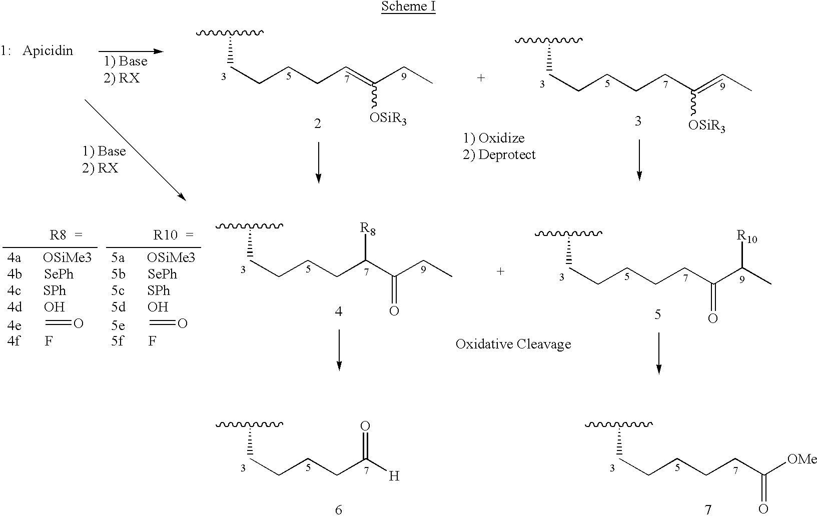

Apicidin is first enolized with an appropriate amine base including, but not limited to, LiN(iPr)₂, NaN(SiMe₃)₂, KN(SiMe₃)₂, and the like at temperatures ranging from −78° C. to 0° C. to form an enolate. The amine base is preferably KN(SiMe₃)₂. Appropriate solvents for this reaction include, but are not limited to, Et₂O, dioxane, tetrahydrofuran (THF), dimethoxyethane, and the like. The solvent is preferably THF. The enolate is reacted with an appropriate electrophile RX including, but not limited to, MeI, EtI, allyl bromide, benzyl bromide, PhSeCl, PhSCl, PhSSPh, (MeO)₂P(O)Cl, (CF₂SO₂)₂O, Et₃SiCl, tBu(Me)₂SiCl, (nPr)₃SiCl, Me₃SiCl, Ph(Me)₂SiCl, and the like to form a silyl enol ether. The electrophile is preferably Me₃SiCl.

Treatment of the thus prepared silyl enol ethers with an oxidant, including but not limited to, H₂O₂, tBuOOH, Me₃SiOOH, AcOOH, dimethydioxirane and the like, or preferably MCPBA (meta-chloroperbenzoic acid), at temperatures from −78° C. to RT (room temperature) but preferably 0° C. to RT will produce the corresponding alpha-silyloxyketones, compounds 4a/5a. The silyl protecting groups can be then removed using a variety of acid or fluoride sources including, but not restricted to, HCl, H₂SO₄, HBF₄, acetic acid, PPTS (pyridinium p-toluenesulfonate), TsOH (p-toluenesulfonyl hydroxide), HF, HF.pyridine, or nBu₄NF and the like in protic or aprotic solvents including, but not limited to, CH₂Cl₂, CHCl₃, MeOH, EtOH, iPrOH, THF, Et₂O and dioxane and the like at temperatures from 0° C. to 50° C. to generate the alpha-hydroxyketones, compounds 4d/5d.

The alpha-hydroxyketone compounds 4d/5d may be separated or used with no further separation, as desired. Compounds 4d/5d can be oxidized to the corresponding diketones, compounds 4e/5e, by treatment including, but not limited to, Swern oxidation, Dess-Martin oxidation, PCC (pyridinium chlorochromate), PDC (pyridinium dichromate), Moffat-oxidation, and the like, or most preferably TPAP/NMO (tetrapropylammonium perruthenate(VII)/4-methylmorpholine N-oxide) in solvents including, but not limited to, toluene, CH₂Cl₂, CHCl₃ and the like at temperatures ranging from −78° C. to RT.

The alpha-hydroxyketone compounds 4d/5d can be converted into the corresponding alpha-haloketone compounds such as 4f/5f by treatment with Ph₃P/CBr₄, Ph₃P/I₂, PH₃P/CCl₄, Ph₃P/CHCl₂CHCl₂, DAST (diethylaminosulfur trifluoride), morpholinyl sulfur trifluoride, and the like in solvents such as CH₂Cl₂, CHCl₃, benzene, toluene and the like at temperatures from −78° C. to RT.

The alpha-hydroxyketone compounds 4d/5d can be treated with an oxidizing agent including, but not restricted to, NaIO₄, HIO₄, MnO₂, Amberlite® IRA-904 ion-exchange resin available from Aldrich Chemical Company, Milwaukee, Wis., NaIO₄, KIO₄, and nBu₄NIO₄, or most preferably Pb(OAc)₄ to yield a C7-aldehyde compound 6, and a C8-methyl ester compound 7, by an oxidative cleavage reaction. The oxidative cleavage reaction may be performed in a variety of solvents or mixtures of solvents, including water, EtOH, iPrOH (isopropanol), tBuOH (tert-butanol), acetone, ether, THF, benzene, toluene, CH₂Cl₂, CHCl₃, and the like, or most preferably MeOH. Generally, the oxidative cleavage reaction is performed at temperatures from about −78° C. to about 80° C. When utilizing MeOH, the reaction should be performed at temperatures from −20° C. to RT. The oxidative cleavage reaction may be improved by the addition of a base, including but not restricted to NaHCO₃, Et₃N, EtN(iPr)₂, lutidine and the like, or most preferably pyridine. The oxidative cleavage reaction is generally complete in from about 5 minutes to about 24 hours.

Referring to Scheme II below, the phenylsulfide compounds 4c/5c or phenylselenide compounds 4b/5b, analogs of apicidin, are oxidized to the corresponding sulfoxide or selenoxide compounds (not shown) using reagents which include, but not limited to, Oxone, MCPBA, tBuOOH, AcOOH, NaIO₄, dimethyldioxirane, and the like, or most preferably H₂O₂, in solvents or mixtures of solvents, including, but not limited to toluene, CHCl₃, MeOH, water, or most preferably CH₂Cl₂ and at temperatures ranging from −20° C. to 50° C.

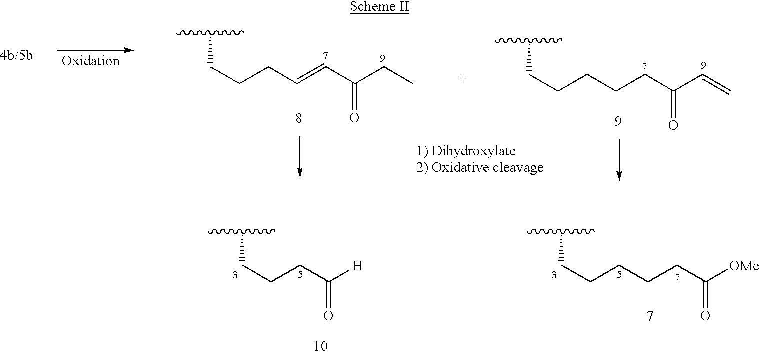

Although the Scheme II shows only compounds 4b/5b as the starting compounds, the same scheme applies just as well to using compounds 4c/5c as starting compounds. The sulfoxides and selenoxides are thermally eliminated to generate the corresponding enone compounds 8 and 9 in solvents including, but not limited to, CH₂Cl₂, CHCl₃, MeOH, or most preferably toluene, at temperatures ranging from RT to 110° C.

Enone compounds 8 and 9 can be epoxidized (not shown) with appropriate epoxidizing agents including, but not limited to, dimethyldioxirane, H₂O₂, tBuOOH, AcOOH, and the like, or most preferably MCPBA, in solvents or mixtures of solvents including, but not limited to, toluene, CHCl₃, MeOH, or most preferably CH₂Cl₂, at temperatures ranging from −20° C. to RT.

Enone compounds 8 and 9 also may be dihydroxylated with OsO₄ under conditions known to those skilled in the art to form the corresponding diols. Osmium tetroxide may be used either stoichiometrically or catalytically in the presence of an oxidant including, but not restricted to, morpholine N-oxide, trimethylamine N-oxide, hydrogen peroxide, tert-butyl hydroperoxide and the like. The dihydroxylation reactions are performed in a variety of solvents or mixtures of solvents. The solvents include both protic and aprotic solvents such as water, MeOH, EtOH, tert-butanol, ether, TEF, benzene, pyridine, acetone, and the like. The dihydroxylation reactions are performed at from −78° C. to 80° C. and are complete in from 5 minutes to 24 hours. The diol products thus obtained can be oxidatively cleaved as described previously for compounds 6 and 7 to yield a C₆-aldehyde compound 10 and a C8 methyl ester compound 7 from compounds 8 and 9, respectively.

Referring to Scheme III below, apicidin's sidechain C8-ketone group can be a starting point for analog synthesis.

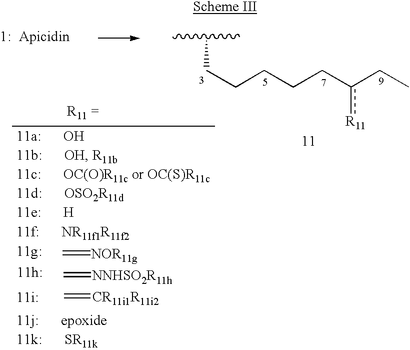

R_{11b}, R_{11c}, R_{11d}, R_{11f1}, R_{11f2}, R_{11g}, R₁₁ₕ, R₁₁ᵢ₁, R₁₁ᵢ₂, and R₁₁ₖ are each independently an alkyl or aryl group which optionally is substituted.

By Scheme III, the sidechain C8-ketone group can be reduced using reagents known to those skilled in the art, including, but not limited to LiBH₄, LiAlH₄, DIBAL-H (diisobutylaluminum hydride), K-Selectride® (potassium tri-sec-butylborohydride) available from Aldrich Chemical Company, Milwaukee, Wis., L-Selectride® (lithium tri-sec-butylborohydride) available from Aldrich, Alpine-Borane® (B-isopinocampheyl-9-borabicyclo[3.3.1]-nonane) available from Aldrich, and the like or most preferably NaBH₄ to yield the C8 alcohol compound 11a. These reduction reactions may be performed in protic or aprotic solvents including, but not limited to, THF, ether, dimethyl ether, dioxane, EtOH, CH₂Cl₂, EtOAc, CHCl₃, benzene, toluene, or most preferably MeOH, and at temperatures from −78° C. to RT.

Apicidin's sidechain C8-ketone group can also be treated with RMgBr, RMgCl, RMgI, RLi, R₂CuLi, RCeCl₂Li and the like to generate substituted alcohol compounds 11b. In these RLi, RLiX, or RMgX type reactants, R is an alkyl or aryl group, and the alkyl and aryl groups are optionally substituted. These substitution reactions may be performed in solvents or mixtures of solvents, including but not limited to, Et₂O, dioxane, HMPA (hexamethylphosphoramide), DMSO, NMP (1-methyl-2-pyrrolidinone), dimethoxyethane, and the like, or most preferably THF, at temperatures from −78° C. to RT, and are complete in from 5 minutes to 12 hours.

The C8-alcohol compound 11a generated above can be alkylated, acylated or sulfonylated using known methods for acylation, sulfonylation and alkylation of alcohols to generate apicidin derivative compounds 11c or 11d. Thus, acylation may be accomplished using reagents such as acid anhydrides, acid chlorides, chloroformates, carbamoyl chlorides, ClC(S)OPh(F₅), thiocarbonyldimidazole, isocyanates, and the like, and amine bases according to general procedures known to those skilled in the art. Sulfonylations may be carried out using sulfonyl chlorides or sulfonic anhydrides. Alkylations may be carried out using alkyl halides or trichloroacetimidates. Suitable solvents for these reactions include benzene, toluene, CHCl₃, CH₂ClCH₂Cl, and the like, or most preferably CH₂Cl₂, and may be performed from temperatures of 40° C. to 80° C.

The hydroxyl group at C8 of compound 11a can be eliminated using Burgess reagent, Martin's sulfurane reagent or by treating compound 11d with a base to generate a mixture of C6, C7- and C7, C8-olefin isomers. Suitable bases include, but are not limited to, Et₃N, EtN(iPr)₂, NaOMe, KOtBu, and the like or most preferably DBU in solvents such as CH₂Cl₂, CHCl₃, toluene, benzene, MeOH, EtOH, pyridine and the like and at temperatures from 0° C. to 110° C. The C8-hydroxyl group of compound 11a can also be eliminated by reduction via the intermediary compound 11c wherein R is OPh, OPh(F₅), Set, and the like, or most preferably N-1-imidazolyl. Intermediary compound 11c is treated with i) a radical initiator such as oxygen/Et₃B, AIBN (2,2′-azobisisobutyronitrile), benzoyl peroxide and the like, and ii) a hydride source, including, but not limited to, Et₃SiH, Me₃SnH, Ph₃SnH, Ph₃AsH, nBu₃SnCl/NaBH₄, and the like, or most preferably nBu₃SnH in solvents including but not limited to CH₂Cl₂, CHCl₃, benzene, MeOH, EtOH, or most preferably toluene, and the like, at temperatures from −78° C. to 110° C., to form compound 11e.

Apicidin can be treated with mono- or disubstituted amines, a hydride source, and a proton source to generate compound 11f. Suitable solvents include, but are not restricted to, benzene, toluene, EtOH, iPrOH and the like, or more preferably, MeOH. Suitable proton sources include, but are not limited to, TsOH, HCl, HCO₂H, PPTS and the like, or most preferably HOAc. The intermediate imine may be reduced in situ as it is formed or after azeotropic removal of water using a Dean-Stark trap. Suitable reducing agents include, but are not limited to, LiAlH₄, NaBH₄, LiBH₄, H₂/(10% Pd/C) and the like, or most preferably NaBH₃CN.

Oxime compound 11g and hydrazone compound 11h are prepared by treating apicidin with hydrazine in a solvent with a proton source. For example, apicidin can be treated with mono- or disubstituted amines, and a proton source. Suitable solvents include, but are not restricted to, benzene, toluene, EtOH, iPrOH and the like, or more preferably, MeOH. Suitable proton sources include, but are not limited to, TsOH, HCl, HCO₂H, PPTS and the like, or most preferably HOAc.

Apicidin is treated with stabilized Wittig reagents, unstabilized Wittig reagents or Homer-Emmons reagents to generate the unsaturated product, compound 11i. Suitable reagents include, but are not limited to, Ph₃P═CH₂, Ph₃P═CHMe, Ph₃P═CH(nPr), (MeO)₂P(O)CH₂CO₂Me, Ph₃P═CH₂C(O)Me and the like. These olefination reactions may be performed in solvents including, but not limited to, DMF (N,N-dimethylformamide), MeOH, CH₂Cl₂, toluene, Et₂O, MeCN, THF and the like and may be performed at from −78° C. to 110° C. The C8 ketone of apicidin may be converted into an epoxide (compound 11j) by treated with CH₂═N₂ or Me₃SiCH═N₂ in MeOH, or Me₃S(O)I in a solvent such as tBuOH, dimethoxyethane, THF, DMF, DMSO, or more preferably HMPA and a strong base such a tBuOK, nBuLi, or more preferably NaH at temperatures from −78° C. to 50° C.

Treatment of compound 11d with an appropriate sulfur containing nucleophile permitted the introduction of sulfur at C8 to form compound 11k. Suitable nucleophiles include NaSMe, KSAc, HSPh/Et₃N, HSCH₂CH₂OH/EtN(iPr)₂ and the like. These reactions proceed readily in polar solvents such as MeOH, EtOH, DMF, DMSO, HMPA, NMP and the like at temperatures from 0° C. to 50° C.

Referring to Scheme IV below, a Beckmann rearrangement to form compounds 12a and 12b can be induced by treatment of compound 11g with an acylating agent, including but not limited to, POCl₃, SOCl₂, MeSO₂Cl and the like or more preferably TsCl and an amine base at temperatures from 0° C. to 50° C. Suitable amine bases include Et₃N, EtN(iPr)₂, lutidine, DBU (1,8-diazabicyclo[5.4.0]undec-7-ene) and the like, or most preferably pyridine. Pyridine also may serve as a solvent for this reaction or alternatively MeCN, benzene, toluene, dioxane and the like may be used.

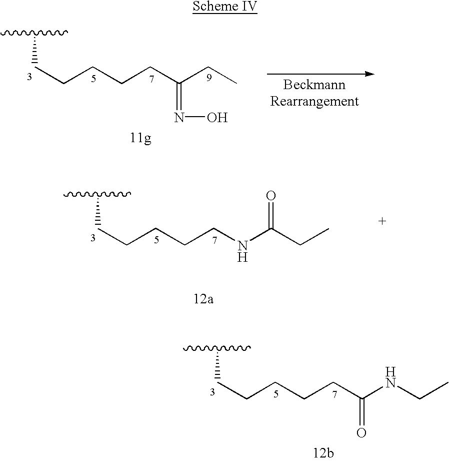

Referring to Scheme V below, the C7-aldehyde compound 6 could be oxidized to the corresponding C7 methyl ester compound 13 by treating with suitable oxidants including NaOCl/HOAc/MeOH, tBuOCl/MeOH/pyridine, and the like, or most preferably PDC/DMF/MeOH under conditions known in the art. The C7 methyl ester compound 13 can further serve as the starting material for additional derivatives. Similarly, the C₆-aldehyde compound 10 can be oxidized to its corresponding C6 methyl ester (not shown).

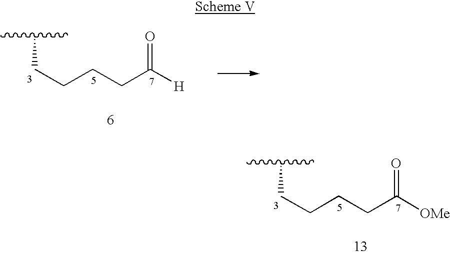

Referring to Scheme VI below, the methyl ester compounds 7 and 13 can be converted into a series of esters, amides and ketones.

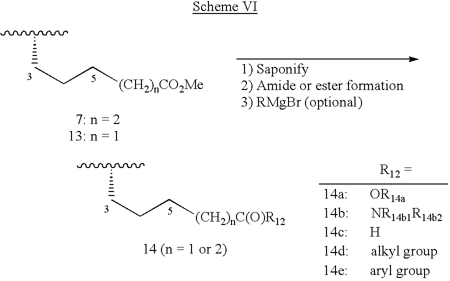

R₁₄ₐ, R_{14b1}, and R_{14b2}, is each independently an alkyl or aryl group, which optionally is substituted.

Saponification could be accomplished by treating compound 7 with reagents including, but not limited to, NaOH, KOH, Me₃SiOOK, LiOOH and the like, or more preferably LiOH. Solvents, or mixtures of solvents, include MeOH, EtOH, tBuOH, DMF, DMSO, HMPA, Et₂O, THF, water and the like. The reaction proceeds at temperatures from 0° C. to 100° C. Amide and ester formation may be accomplished by reacting the C8-carboxylic acid (compound 14a) thus prepared using standard ester- and amide-forming reagents known to those skilled in the art. The esterification reaction is carried out using at least one equivalent of an alcohol, HOR. Although preferably ten to one hundred equivalents of alcohol are used, the esterification also may be carried out using the alcohol as solvent. Esterification reagents include, but are not restricted to, dicyclohexylcarbodiimide, 1-(3-dimethylaminopropyl)-3-ethylcarbodiimide hydrochloride (EDC.HCl), diisopropylcarbodiimide, benzotriazol-1-yloxy-tris(dimethylamino)phosphonium hexafluorphosphate (BOP), bis(2-oxo-3-oxazolidinyl)phosphinic chloride (BOP-Cl), benzotriazole-1-yl-oxy-tris-pyrrolidino-phosphonium hexafluorophosphate (PyBOP), chloro-tris-pyrrolidino-phosphonium hexafluorophosphate (PyClOP), bromo-tris-pyrrolidino-phosphonium hexafluorophosphate (PyBrOP), diphenylphosphoryl azide (DPPA), 2-(1H-benzotriazole-1-yl)-1,1,3,3-tetramethyluronium hexafluorophosphate (HBTU), O-benzotriazol-1-yl-N,N,N′,N′-bis(pentamethylene)uronium hexafluorophosphate and 2-chloro-1-methylpyridinium iodide. The ester-forming reactions may be facilitated by the optional addition of N-hydroxybenzotriazole, N-hydroxy-7-aza-benzotriazole, 4-(N,N-dimethylamino)pyridine or 4-pyrrolidinopyridine. The ester-forming reaction is generally performed using at least one equivalent (although several equivalents may be employed) of amine bases such as triethylamine, diisopropylethylamine, pyridine and the like. The carboxyl group may be activated for ester bond formation via its corresponding acid chloride or mixed anhydride, using conditions known to those skilled in the art. The ester-forming reaction is carried out in an aprotic solvent such as, for example, methylene chloride, tetrahydrofuran, diethyl ether, dimethylformamide, N-methylpyrrolidine, and the like, at temperatures ranging from −20° C. to 60° C., and is complete in about 15 minutes to about 24 hours. Amides (where R₁₂ is NR_{14b1}R_{14b2}) are prepared as described for esters (vida supra) from the corresponding carboxylic acids using and an appropriate amine, HNR_{14b1}R_{14b2}.

The amide compound 14b (in which NR_{14b1}R_{14b2} is N(OMe)Me) can be treated with nucleophilic agents to yield the corresponding aldehyde (compound 14c) and ketones (compounds 14d and 14e). Suitable nucleophiles include, but are not limited to, hydride reagents, RLi or RMgX and the like as described above for the preparation of compounds 11a and 11b. In addition, the aldehyde and ketone products 14c, 14b and 14e can be further reacted with hydride reagents, RLi or RMgX, to generate the corresponding alcohol adducts as described previously.

Referring to Scheme VII below, the aldehyde compounds 6, 10 and 14c serve as starting material for the preparation of a variety of derivatives.

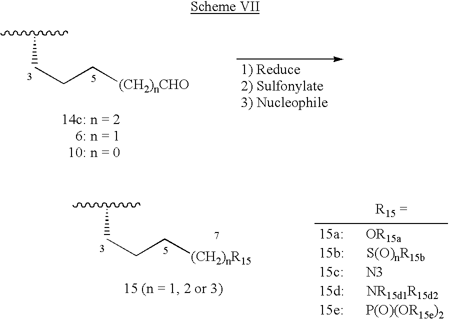

R₁₅ₐ, R_{15b}, R_{15d1}, R_{15d2}, and R₁₅ₑ, are each independently an alkyl or aryl group which optionally is substituted.

Reduction of the side chain aldehyde group in compounds 6, 10 and 14c with hydride reagents produced compound 15a (where R₁₅ₐ=H). The side chain alcohol thus obtained can then be sulfonylated, as described above in Scheme III. The sulfonyl group can then be displaced with an appropriate sulfur, nitrogen or phosphorous nucleophile to form compounds 15b, 15c and 15e respectively. Suitable nucleophiles include NaSMe, KSAc, NaN₃, (PhCH₂O)₂P(O)H, (P(OCH₂Ph)₃, (MeO)₂P(O)H, P(OMe)₃ and the like.

Further, the side chain azide compound 15c can be reduced using conditions known to those skilled in the art including, but not restricted to, H₂/10% Pd/C, HSAc/MeOH, SnCl₂, Ph₃P/H₂O and the like to form a side chain amine compound (not shown). The amine compound thus obtained can be acylated, alkylated or sulfonylated as described above. Alternatively, reductive amination of the aldehyde compounds 6, 10 and 14c with a suitable amine as described above will generate the amine compound 15d.

Referring to Scheme VIII below, the side chain of compounds 6, 10 or 14c can be extended by reacting the aldehyde with stabilized Wittig reagents, unstabilized Wittig reagents or Homer-Emmons reagents to form compound 16a.

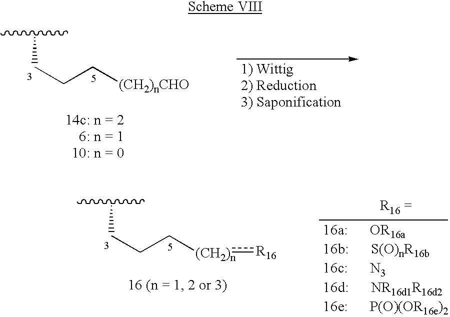

R₁₆ₐ, R_{16b}, R_{16d1}, R_{16d2}, and R₁₆ₑ are each independently an alkyl or aryl group, which optionally is substituted.

The side chain unsaturation of compound 16a can be reduced by catalytic hydrogenation using conditions known to those skilled in the art. Suitable catalysts include 5% Pd/C, 10% Pd/C, Pd(OH)₂, PtO₂, RhCl₃, RuCl₂(PPh₃)₃, and the like. The hydrogenation reactions may be performed in solvents or mixtures of solvents including CH₂Cl₂, CHCl₃, toluene, MeOH, EtOH, EtOAc, acetone, THF, Et₂O, dimethoxyethane, DMF, DMSO, and the like. The reductions may be run at from one to 10 atmospheres of hydrogen pressure and the reactions are complete in from 5 min to 24 h. For apicidin analog compounds 16a or 16b in which R₁₆ₐ or R_{16b} represents an ester moiety, the ester may be saponified and the carboxylic acid thus obtained may be converted into other esters or amides as described previously.

Referring to Scheme IX below, the N-methoxy group of apicidin may be removed by hydrogenation as described previously and the liberated indole nitrogen compound thus generated may be N-alkylated, acylated or sulfonylated using known methods for acylation, sulfonylation and alkylation of indoles to generate apicidin derivative compound 17.

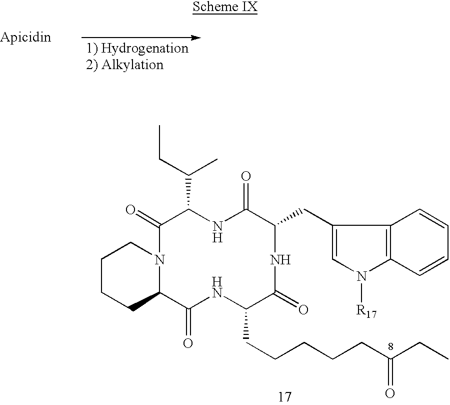

R₁₇ is an alkyl or aryl group, which optionally is substituted.

Thus, acylation may be accomplished using reagents such as acid anhydrides, acid chlorides, chloroformates, carbamoyl chlorides, isocyanates and the like according to general procedures known to those skilled in the art. Sulfonylations may be carried out using sulfonyl chlorides or sulfonic anhydrides. Alkylations may be carried out using alkyl halides. Suitable bases for these acylation, sulfonylation and alkylation reactions include KH, nBuLi, tBuLi, LiN(iPr)₂, NaN(SiMe₃)₂, KN(SiMe₃)₂ and the like or more preferably NaH. Suitable solvents, or mixtures of solvents for these reactions include benzene, toluene, CHCl₃, CH₂ClCH₂Cl, CH₂Cl₂, DMSO, HMPA, NMP and the like or most preferably DMF and may be performed from temperatures of −40° C. to 80° C.

When the newly incorporated R₁₇ group contains an ester moiety, the apicidin derivative compound 17 can be saponified to the corresponding carboxylic acid and converted into a series of amides using conditions described previously.

When the newly incorporated R₁₇ group contains an alcohol function, the apicidin derivative compound 17, can be acylated, alkylated, phosphorylated or sulfonylated as described previously. Alternatively, this alcohol function may be converted into a leaving group such as a sulfonate or halide and displaced with appropriate sulfur, nitrogen or phosphorus nucleophiles as described previously Referring to Scheme X below, apicidin's tryptophan may be allylically oxidized to generate beta-oxo apicidin analog compound 18 using conditions known to those skilled in the art. (What is R₁₈?)

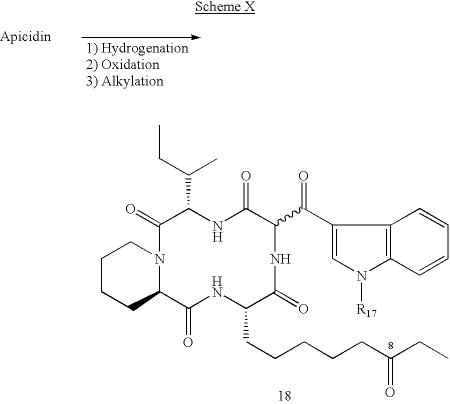

R₁₈ is an alkyl or aryl group, which optionally is substituted.

Suitable oxidants include but are not restricted to tBuOOH, SeO₂, CrO₃, Na₂CrO₄, PCC, and the like, or more preferably DDQ (2,3-dichloro-5,6-dicyano-1,4-benzoquinone). Appropriate solvents, or mixtures of solvents, include DMF, toluene, benzene, CH₂Cl₂, CHCl₃, HOAc, pyridine, THF, MeOH, EtOH, water, and the like, or more preferably MeCN. These reactions are performed at from −20° C. to 50° C. and are complete in from 5 min to 24 h. The stereochemistry of the beta-oxo-tryptophan attachment of compound 18 may be changed by treatment with bases such as pyridine, EtN(iPr)₂, NaH, KH, DBU, lutidine, or most preferably Et₃N. The epimerization reaction proceeds at from 0° C. to 50° C. in solvents including CHCl₃, CH₂ClCH₂Cl, MeOH, EtOH, DMF, DMSO, NMP, and the like, or most preferably CH₂Cl₂. The nitrogen of the beta-oxo-tryptophan may be alkylated, acylated, sulfonylated or phosphorylated as described previously.

The beta-oxo carbonyl of compound 18 may be selectively reduced using a hydride source under radical conditions. Suitable hydride sources include Me₃SnH, nBu₃SnCl/NaBH₄, Ph₃SnH, Ph₃AsH, and the like, or most preferably nBu₃SnH, in the presence of radical initiators. Suitable radical initiators include, for example, benzoyl peroxide, Et₃B/O₂, and the like, or most preferably AIBN. Suitable solvents for the carbonyl reduction include MeOH, EtOH, water, benzene, or most preferably toluene. The reaction proceeds at temperatures from 0° C. to 110° C.

Referring to Scheme XI below, the indole of apicidin may be subjected to oxidative degradation to prepare carboxylic acid compound 19a (where R₂=OH) using conditions known to those skilled in the art.

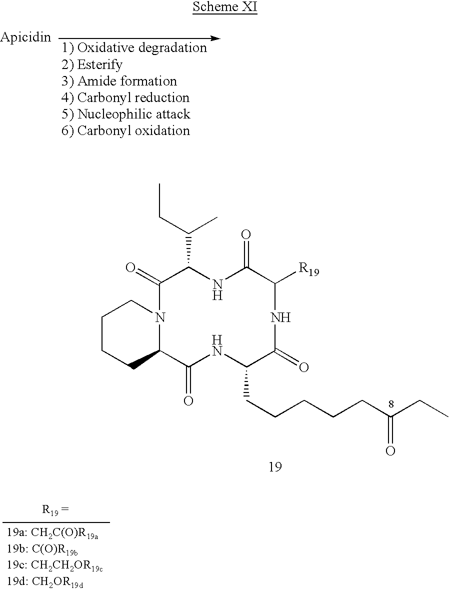

R₁₉ₐ, R_{19b}, R_{19c}, and R_{19d} are each independently an alkyl or aryl group, which optionally is substituted.

Suitable oxidants include, but are not restricted to, KMnO₄, KMnO₄/NaIO₄, NaIO₄/RuO₄, and the like, or most preferably NaIO₄/RuCl₃. Suitable solvents, or mixtures of solvents include CHCl₃, CH₂ClCH₂Cl, MeCN, MeOH, EtOH, tBuOH, and the like, or most preferably CH₂Cl₂. The reaction proceeds at temperatures from 0° C. to 50° C. This carboxylic acid may be converted into esters or amides as described previously. Alternatively, a methyl ester may be prepared first (eg. compound 19a, wherein R₁₉ₐ is OMe) and reacted with LiN(OMe)Me, Me₂AlN(OMe)Me, or most preferably BrMgN(OMe)Me, to produce a Weinreb amide compound 19a, in which R₁₉ₐ is N(OMe)Me. Suitable solvents for this reaction include Et₂O, dimethoxyethane, dioxane, and the like, or most preferably THF. The reactions are performed at from −78° C. to 50° C. and are complete in from 30 min to 12 h.

Reduction of the sidechain C8-ketone group of compound 19a to the corresponding alcohol proceeds as described previously. The Weinreb amide thus directly generated can then be reacted with hydride reagents, RLi, or RMgX as described previously to prepare the corresponding aldehyde or ketones (eg. 19a where R₁₉ₐ is H, alkyl or aryl group). At this point, the side chain C8-alcohol may be oxidized back to regenerate the C8-ketone as described previously.

When R₁₉ₐ is OH in compound 19a, the carboxylic acid may be reduced using BH₃ to form an alcohol compound 19c (where R_{19c} is H). This alcohol may be acylated, sulfonylated or phosphorylated as described previously. Treatment of the alcohol compound 19c with Ar₃Bi reagents will generate the corresponding aryl ether compound 19c in which R_{19c} is an aryl group. Both the alpha- and beta-stereoisomers at the tetrapeptide are accessible as described previously.

Substitution of beta-oxo apicidin derivative compound 18 for apicidin in Scheme XI above results in the formation of the truncated apicidin analog compounds 19b and 19d.

Referring to Scheme XII, the 2,3-indole bond in Apicidin can be cleaved oxidatively to form compound 20 using conditions known to those skilled in the art.

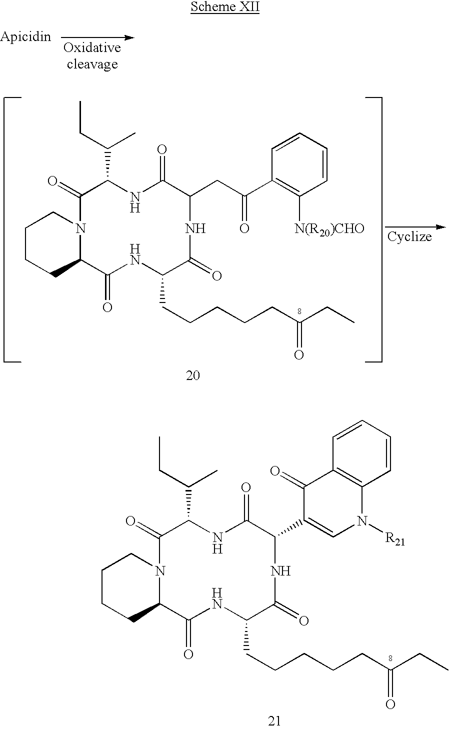

R₂₀ and R₂₁ are each independently an alkyl or aryl group which optionally is substituted.

Suitable oxidants include KMnO₄, NaIO₄, Pb(OAc)₄, and the like, or more preferably ozone. This reaction may be run in solvents such as CHCl₃, CH₂ClCH₂Cl, and the like, or more preferably CH₂Cl₂, at temperatures from −78° C. to RT and the reaction is complete in from 1 min to 2 h. Treatment of compound 20 with a base induces Aldol cyclization to form a quinolone compound 21. Suitable bases for this reaction include Et₃N, EtN(iPr)₂, pyridine, DBU, NaOMe, NaOEt, NaHCO₃, and the like, or more preferably KOtBu. The Aldol cyclization may be performed in solvents, or mixtures of solvents including CH₂Cl₂, CHCl₃, MeOH, EtOH, DMF, THF, Et₂O, DMSO, water, and the like, or more preferably tBuOH. The reaction is complete in from 10 min to 12 h at 0° C. to RT. Substitution of N-substituted-N-desmethoxy-apicidin derivatives (Compound 17) for apicidin in Scheme XII leads to the formation of N-substituted quinolone derivatives.

Referring to Scheme XIII below, the quinolone compound 21 can be treated with sulfonylating agents as described previously to form compound 22 wherein R₂₂ is a sulfonate moiety

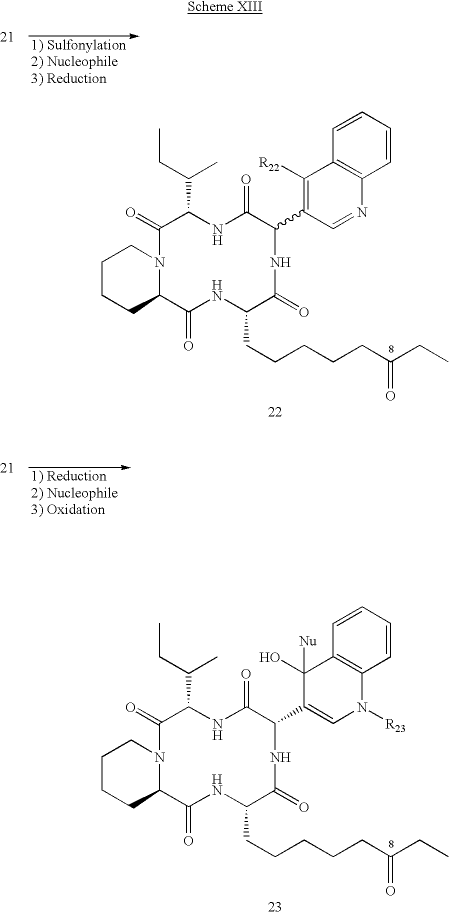

R₂₂ and R₂₃ are each independently an alkyl or aryl group, which optionally is substituted.

During this reaction, some inversion of stereochemistry at the tetrapeptide ring juncture occurs. When R₂₂ of compound 22 is OSO₂CF₃, the triflate can be displaced with suitable nucleophiles, such as halogen, sulfur nucleophiles or nitrogen nucleophiles including, but not limited to, NaBr, NaCl, KI, NaN₃, NaSMe, KSAc, pyridine and the like. The resulting compounds are not shown. Suitable solvents for the displacement reaction include, but are not limited to, CH₂Cl₂, CHCl₃, DMF, DMSO, HMPA, NMP, and the like. The reactions proceed at temperatures from 0° C. to 80° C.

For apicidin derivative compound 22 in which R₂₂ is N-1-pyridinium, the pyridinium group may be reduced using catalytic hydrogenation as described previously.

Further, the C8-ketone group of apicidin derivative compound 21 may be reduced first. The thus formed quinolone carbonyl can then be reacted with nucleophiles such as hydride reagents, RLi or RMgX as described previously. The apicidin derivative compound 23 can be prepared by reoxidation of the C8-alcohol as described previously.

Referring to Scheme XIV below, apicidin may be brominated at the indole C2 position following removal of the N-methoxy group using conditions known to those skilled in the art to form compound 24 where R₂₄ is Br.

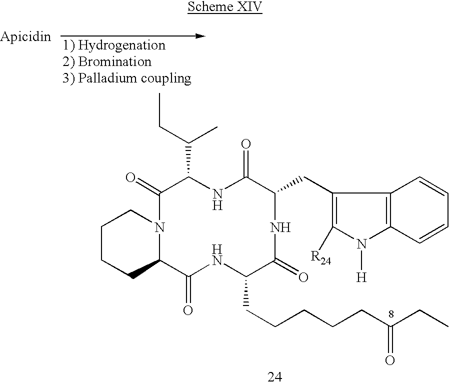

Suitable brominating agents include, but are not limited to, Br₂, Hg(OAc)₂/Br₂, CBr₄, CuBr₂, HOBr, Br₂HOAc/NaOAc, and the like, or most preferably N-bromosuccinamide. The bromination reaction can be facilitated by a radical initiator such as benzoyl peroxide, Et₃B/O₂ or AIBN.

The 2-bromo-indole thus obtained can be further reacted with a palladium catalyst, a base and ArX to induce an aryl coupling reaction. Suitable palladium catalysts include, but are not limited to, Pd(OAc)₂, Pd(OAc)/PPh₃, PdCl₂(PPh₃)₂, Pd(dba)₂/PPh₃, and the like, or most preferably Pd(PPh₃)₄. Suitable bases for this reaction include, but are not limited to, KOtBu, CsCO₃, or most preferably NaHCO₃. Suitable solvents, or mixtures of solvent for this coupling reaction include toluene, DMF, MeCN, NMP, DMSO, H₂O, EtOH, or most preferably dioxane/water. Suitable ArX groups include, but are not limited to, PhB(OH)₂, 2-napthylboronic acid, (4-Me)PhB(OH)₂, (4-F)PhOTf, and the like. The reactions are complete in from 30 min to 48 h at temperatures from RT to 110° C.

### Synthesis of Side Chain Modified Apicidin Derivatives

In the Examples, and elsewhere herein, all percentages are by weight unless specifically stated otherwise. Further, all ratios of compounds are by volume unless specifically stated otherwise. Room temperature (RT) means a temperature from about 18° C. to about 25° C. If no temperature is specified, then the conditions are understood to be room temperature. In certain steps that describe using an ingredient without specifying an amount, one of ordinary skill would understand the desired result and can determine the amount without difficulty. In general, the purities of the pure Examples were better than about 95% pure.

### EXAMPLE 1

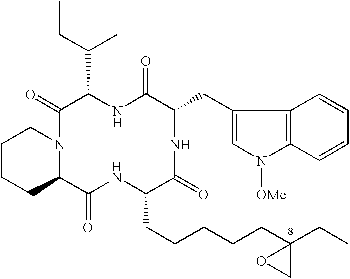

Example 1 was prepared by the following procedure. At room temperature, 27 mg of Me₃S(O)I was added to a mixture of i) 5.6 mg of 60% NaH and ii) 0.35 mL HMPA. The resulting solution was allowed to stand for 5 min. Then, a mixture of 12 mg apicidin in 96 μL DMF was added to form a reacting mixture. After 48 hours, the reaction was quenched with water, extracted with EtOAc and dried in Na₂SO₄ to produce 8 mg Example 1. Example 1 was thus obtained without requiring further purification and was characterized by ¹H NMR and MS [m/z: 638 (M⁺+1)].

### EXAMPLE 2

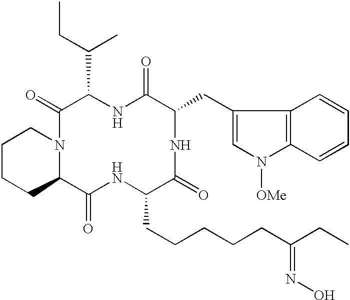

Example 2 was prepared by the following procedure. At room temperature, 60 mg HCl.H₂NOH and 181 μL Et₃N was added to 20 mg apicidin in 10 mL CH₂Cl₂. The resulting solution was aged for 12 h. The volatiles were then removed under reduced pressure. Example 2 was obtained following preparative RP-HPLC (reversed phase high performance liquid chromatography), without workup, using a gradient elution characterized by 1:3 MeCN:H₂O to 100% MeCN, with a 60 min linear ramp. The pure Example 2 thus obtained was characterized by ¹H NMR and MS [m/z: 639.3 (M⁺+1)].

### EXAMPLES 3A-3M

Examples 3a-3m were prepared following the general procedure described in Scheme III for compound 11f, 11g, and 11h, and for Ex. 2. Examples 3a-3m are described by the following chemical formula and were characterized by NMR and mass spectroscopy:

TABLE 1

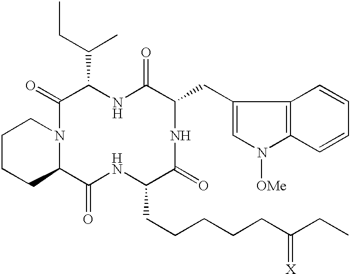

| Example | X Group | Mass Spec |
|---|---|---|
| Ex. 3a | NNHSO₂Ph(4-Me) | — |
| Ex. 3b | NOCH₂Ph | 729.2 (M⁺ + 1) |
| Ex. 3c | NNH-Dansyl | 871.2 (M⁺ + 1) |
| Ex. 3d | NOCH₂CO₂—Na+ | — |
| Ex. 3e | NOCH₂CO₂H | 697.2 (M⁺ + 1) |
| Ex. 3f | NOMe | 653.2 (M⁺ + 1) |
| Ex. 3g | NNH-Texas Red | 1227.2 (M⁺ + 1) |
| Ex. 3h | NOCH₂C(O)NHCH₂CH₂OH | — |
| Ex. 3I | NOCH₂C(O)(N-1-pyrrolindinyl) | — |
| Ex. 3j | NOCH₂CO₂Me | — |
| Ex. 3k | NOC(O)Ph | — |
| Ex. 3l | NOC(O)Me | — |
| Ex. 3m | NOC(O)tBu | — |

### EXAMPLES 4A AND 4B

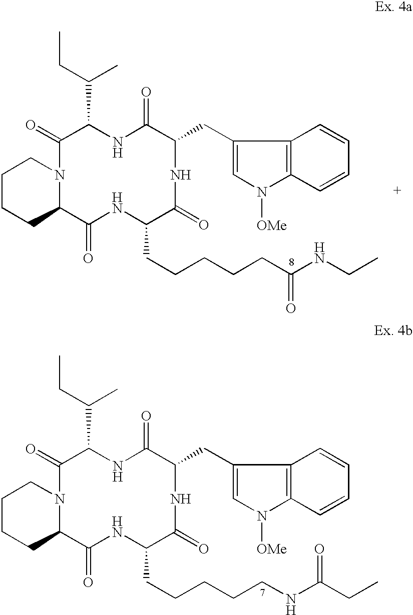

Examples 4a and 4b were prepared by the following procedure. At 0° C., 4.5 mg of p-toluenesulfonyl chloride was added to 3 mg of Example 2 (the C8-ketoxime of apicidin) in 0.5 mL pyridine to form a solution. The solution was maintained at 0° C. for 10 min, then warmed to RT and aged for 50 min. Then 1 mL each of saturated brine and saturated NaHCO₃(aq) were added. Next, the solution was extracted with EtOAc and dried with Na₂SO₄. A mixture of pure Examples 4a and 4b was obtained following preparative RP-HPLC using gradient elution (1:3 MeCN:H₂O isocratic for 10 min, then a 75 min linear ramp to 100% MeCN). The pure mixture thus obtained was characterized by ¹H NMR and MS [m/z: 639.2 (M⁺+1)].

### EXAMPLE 5

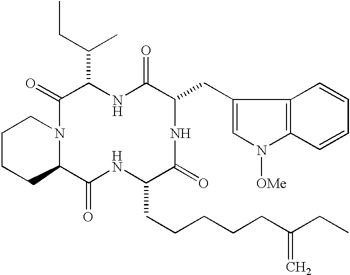

Example 5 was prepared by the following procedure. At room temperature (RT), 114 mg of Ph₃PCH₃⁺Br⁻ was added to i) 16.8 mg of a 60% dispersion of NaH in oil, ii) 2 mL DMF, and iii) 0.2 mL HMPA to form a mixture. After the mixture ceased foaming, a solution of 20 mg apicidin in 1 mL DMF was added. The resulting solution was aged for 4 hours. Preparative RP-HPLC without workup using gradient elution (1:3 MeCN:H₂O isocratic for 10 min, then a 75 min linear ramp to 100% MeCN yielded 14 mg of pure Example 5 which was characterized by ¹H NMR and MS [m/z: 622.3 (M⁺+1)].

### EXAMPLES 6A-6D

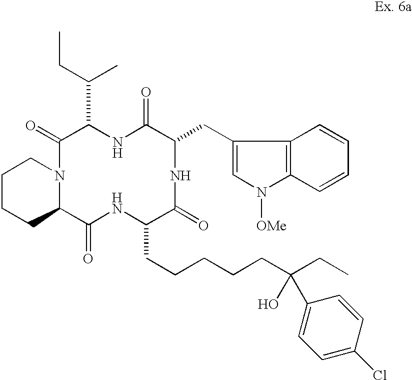

Example 6a was prepared by the following procedure. At 0° C., 0.12 mL of 1.0M (4-Cl)PhMgBr in Et₂O was dropwise added to 15 mg apicidin in a mixture of 1.75 mL THF and 0.25 mL pyridine. After 1 h at 0° C., an additional 0.12 mL of 1.0M (4-Cl)PhMgBr in Et₂O was added. The resulting solution was aged for 1 h at 0° C. and then 1 h at RT. The reaction was quenched by the addition of saturated NH₄Cl(aq) to the solution. The quenched mixture was extracted with EtOAc and dried with Na₂SO₄. Preparative RP-HPLC using gradient elution (1:3 MeCN:H₂O isocratic for 10 min, then a 75 min linear ramp to 100% MeCN) yielded 8 mg of pure Example 6a, which was characterized by ¹H NMR and MS [m/z: 736.3 (M⁺+1)].

Examples 6b, 6c, and 6d are described by the chemical structure shown below. The specific substituents are tabulated in Table 2. Examples 6b, 6c, and 6d were prepared following the general procedure described in Scheme III for compound 11b under conditions similar to those described above for Ex. 6a

TABLE 2

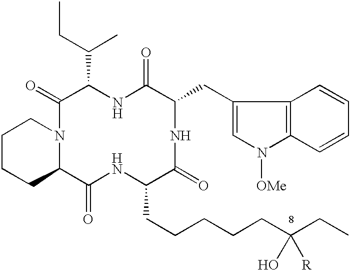

| Example | R Group | Mass Spec |
|---|---|---|
| Ex. 6b | CH₂Ph | 716.4 (M⁺ + 1) |
| Ex. 6c | C₆H₁₁ | 708.4 (M⁺ + 1) |
| Ex. 6d | CH₂CH₃ | 654.4 (M⁺ + 1) |

### EXAMPLE 7

### cyclo(N-O-Methyl-L-Trp-L-Ile-D-Pip-L-2-amino-8-hydroxy-decanoyl)

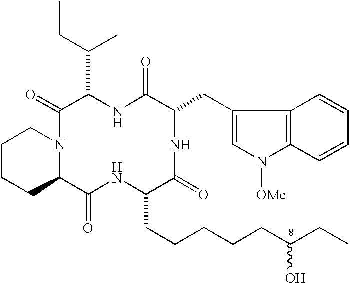

Example 7 was made by first adding 18 mg NaBH₄ to 300 mg apicidin in 12 mL MEOH at 0° C. Next, the ice bath was removed immediately and the solution was stirred at RT for 4 hours. Acetone was added to quench the reaction and the solvents were removed under reduced pressure at ambient temperature. The residue was dissolved in CH₂Cl₂, poured into saturated NaHCO₃, extracted with 1:9 iPrOH:CH₂Cl₂ and dried with Na₂SO₄. The pure product was obtained following flash chromatography on silica gel using 1:1 acetone:hexanes as eluant. The pure Example 7 was characterized by ¹H NMR. TLC: R_{f}=0.32 (1:1 acetone:hexanes).

### EXAMPLE 8

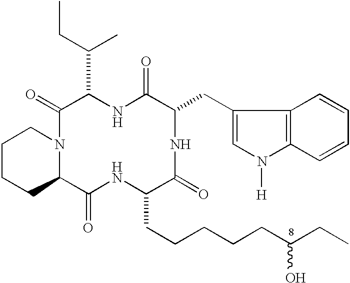

Example 8 was prepared following the general procedure of Example 7 but using N-desmethoxy-apicidin as the starting material. Example 8 was characterized by ¹H NMR and MS [m/z: 596 (M⁺+1)].

### EXAMPLE 9

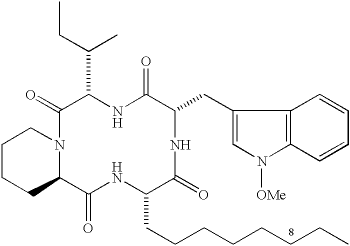

Example 9 was prepared by the following process. At room temperature, 57 mg of thiocarbonylimidazole was added to 40 mg of cyclo(N-O-methyl-L-Trp-L-Ile-D-Pip-L-2-amino-8-hydroxy-decanoyl) in 1.6 mL CH₂Cl₂. The resulting solution was heated to 75° C. for 2 hours. Next, 1 mg of DMAP (4-dimethylaminopyridine) was added and the solution aged for 1 h at 75° C. and 48 h at RT. The solvent then was removed under reduced pressure. 59 mg of the pure intermediary product 8-OC(S)imidazolyl-apicidin (also known as cyclo(N-O-methyl-L-Trp-L-Ile-D-Pip-L-2-amino-8-imidazoylylthionooxy-decanoyl) was obtained by PTLC (2×1500 μm plate) using 1:3:96 NH₄OH:MeOH:CHCl₃ as eluant and was characterized by ¹H NMR and MS [m/z: 736 (M⁺+1)].

To the above prepared 59 mg of intermediary product 8-OC(S)imidazolyl-apicidin in 1.6 mL toluene was added 2.6 mg AIBN and 53 μL nBu₃SnH. The solution was then degassed and heated to 80° C. for 1 h, concentrated under reduced pressure, and partitioned between MeCN and hexanes. The hexanes layer was discarded. The volatiles were removed under reduced pressure and pure Example 9 product was obtained following RP-HPLC using gradient elution (4:6 to 1:0 MeCN:H₂O). Example 9 was characterized by ¹H NMR and MS [m/z: 610 (M⁺+1)].

### EXAMPLE 10

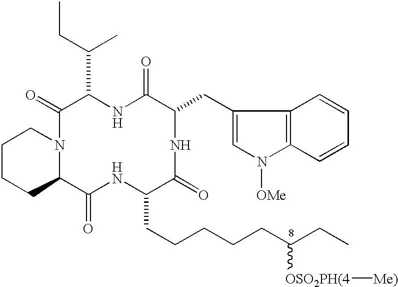

Example 10 was made by adding 10 mg DMAP to 100 mg cyclo(N-O-methyl-L-Trp-L-Ile-D-Pip-L-2-amino-8-hydroxy-decanoyl) in 2 mL pyridine at RT. Next, 94 mg tosic anhydride was added. After 3 d at RT, the solution was poured into saturated NaHCO₃, extracted with CH₂Cl₂ and dried with Na₂SO₄. Pure Example 10 was obtained following flash chromatography on silica gel using gradient elution (1:1:98 then 1:2:97 then 1:3:96 NH₄OH:MeOH:CHCl₃ as eluant). Example 10 was characterized by ¹H NMR. TLC: R_{f}=0.36 (1:3:96 NH₄OH:MeOH:CHCl₃).

### EXAMPLE 11

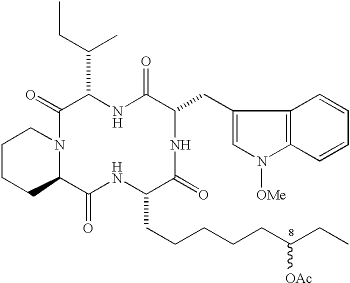

The procedure to form Example 11 was as follows. At room temperature, 50 mg NaBH₄ was added to 100 mg apicidin in 10 mL 1:1 THF:MeOH. After 30 min at RT, the solution was poured into brine, extracted with CH₂Cl₂ and dried with Na₂SO₄. To the residue thus obtained was added 2 mL pyridine, followed by addition of 10 mg DMAP and 10 drops of Ac₂O. After another 15 min at RT, the solution was heated to 80° C. for 10 min. without noting any reaction. An additional 5 drops of fresh Ac₂O (from an unopened bottle) were added and the solution stirred at RT for 24 hours. The solvents were removed under reduced pressure and the residue was lyophilized from dioxane. Preparative RP-HPLC using gradient elution (3:7 to 6:4 MeCN:H₂O) yielded 69 mg of pure Example 11 product, which was characterized by ¹H NMR and MS [m/z: 668.6 (M⁺+1)]. HPLC: t_{R}=4.95 min (6:4 MeCN:H₂O, 1.5 mL/min, Zorbax™ RX-8 available from Rainin Co.

### EXAMPLES 12A-12E

Following the general procedure described in Scheme III, compounds 11c and 11d, and similarly to the procedure for Examples 10 and 11, the following Examples 12a-12f were prepared and characterized by NMR and mass spectroscopy:

TABLE 3

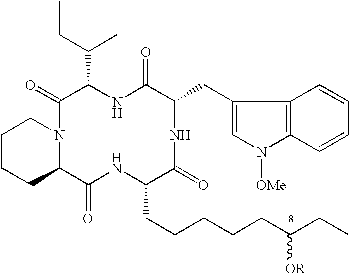

| Example | R Group |
|---|---|
| 12a | C(O)Ph |
| 12b | C(O)tBu |
| 12c | C(O)Ph(F₅) |
| 12d | SO₂Me |
| 12e | SO₂Ph(4-NO₂) |
| 10 | SO₂Ph(4-Me) |

### EXAMPLE 13

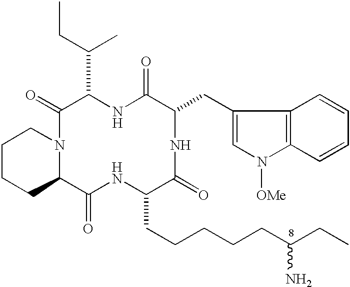

To form Example 13, 0.16 mL (Me₃Si)₂NH and 235 mg ZnCl₂ was added to 100 mg apicidin in 5 mL EtOAc at RT. The solution was heated to 55° C. for 12 hours. The solution was then cooled to 0° C. and 12 mg NaBH₄ was added. After 1 h, the solution was warmed to RT and aged an additional 2 h. The solution was poured into 1:1 brine:saturated NaHCO₃, extracted with CH₂Cl₂ and dried with Na₂SO₄. Pure Example 13 product was obtained following preparative RP-HPLC using gradient elution (3:7 to 6:4 MeCN:H₂O) and was characterized by ¹H NMR and MS [m/z: 625.3 (M⁺+1)]. TLC: R_{f}=0.22 min (1:9:90 NH₄OH:MeOH:CHCl₃).

### EXAMPLE 14

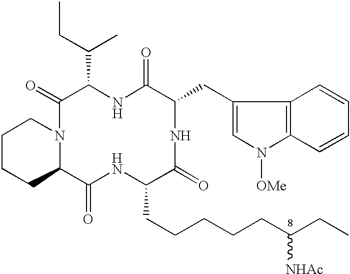

To form Example 14, 2 drops Ac₂O and a catalytic amount of DMAP was added to 14 mg 8-amino-8-desoxo apicidin in 2 mL pyridine at 0° C. The solution was stirred at 0° C. for 30 min and at RT for another 30 min. Next, 1 mL methanol was added and the solution was then concentrated under reduced pressure. Pure Example 14 was obtained following preparative RP-HPLC purification (gradient elution using 25:75 MeCN:H₂O for 10 min, then a 70 min ramp to 100% MeCN) and was characterized by ¹H NMR and MS [m/z: 667.4 (M⁺+1)]. TLC: R_{f}=0.67 (1:9:90 NH₄OH:MeOH:CHCl₃). HPLC: t_{R}=4.60 min, 1:1 MeCN:H₂O, 1.5 mL/min, Zorbax™ RX-8).

### EXAMPLE 15

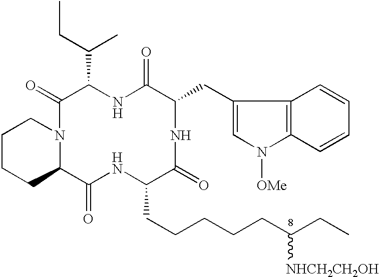

Example 15 was made by first adding to 60 mg apicidin in 0.5 mL MeOH at RT i) 1 mL pyridine, ii) 40 μL ethanolamine, iii) 60 μL glacial HOAc (pH˜5.0), and iv) powdered 4 Å sieves. The solution was cooled to 0° C. and 7.9 mg NaCNBH₃ was added. After 2 h, the solution was warmed to RT and aged for 12 h. The solution was then filtered through Celite filter agent (available from Aldrich Chemical Company, Milwaukee, Wis.) using 1:1 CH₂Cl₂:MeOH as eluant, reduced in volume in vacuo, poured into saturated NaHCO₃, extracted with CH₂Cl₂ and dried with Na₂SO₄. Following preparative RP-HPLC using 1:1 MeCN:H₂O to 100% MeCN gradient elution, 4.2 mg pure Example 15 was obtained. The product thus obtained was characterized by ¹H NMR and MS [m/z: 669 (M⁺+1)].

### EXAMPLE 16

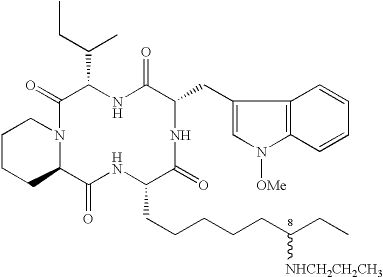

Example 16 was prepared similarly to Example 15. At room temperature, to 60 mg apicidin in 0.5 mL MeOH was added i) 2 mL pyridine, ii) 0.5 mL propylamine, iii) 1 mL glacial HOAc (pH˜4.5), and iv) powdered 4 Å sieves. The solution was cooled to 0° C. and 60 mg NaCNBH₃ was added. After 2 h, the solution was warmed to RT and aged for 12 h. The solution was filtered through Celite using 1:1 CH₂Cl₂:MeOH as eluant, reduced in volume in vacuo, poured into saturated NaHCO₃, extracted with CH₂Cl₂ and dried with Na₂SO₄. Pure Example 16 was obtained following PTLC on silica gel (1×1500 μm plate) using 2:18:80 NH₄OH:MeOH:CHCl₃ as eluant. The pure Example 16 product thus obtained was characterized by ¹H NMR and MS [m/z: 667 (M⁺+1)].

### EXAMPLE 17

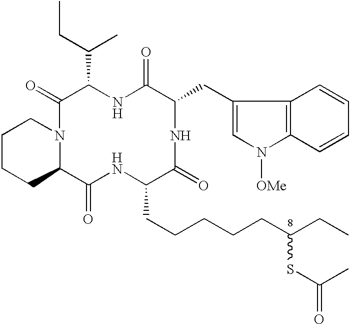

To form Example 17, 32 mg KSAc was added to 18.1 mg of the Example 10 C8-tosylate compound in 3 mL 95% EtOH. The solution was heated to 70° C. for 3 hours. The solution was then cooled to RT and saturated NH₄Cl(aq) was added. Next, the solution was extracted with EtOAc and dried with Na₂SO₄. The solution then was filtered, evaporated to dryness. PTLC on silica gel (1×1000 μm plate) using 3:7 acetone:hexanes as eluant yielded 3.4 mg of pure Example 17 product that was characterized by ¹H NMR.

### EXAMPLE 18

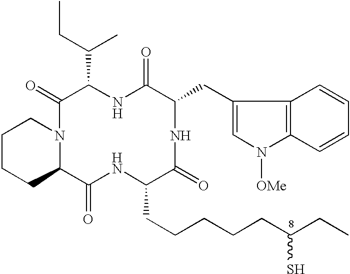

To form Example 18, 3.4 mg of the Example 17 C8-thioacetate compound was placed at RT in 0.2 mL NaOMe 2M solution in MeOH) and aged for 3 h. The solution was poured into saturated NH₄Cl(aq), extracted with CH₂Cl₂, and dried with Na₂SO₄. The solution was filtered, concentrated to dryness, and pure Example 18 was obtained following RP-HPLC. Example 18 thus obtained was characterized by ¹H NMR.

### EXAMPLES 19A AND 19B

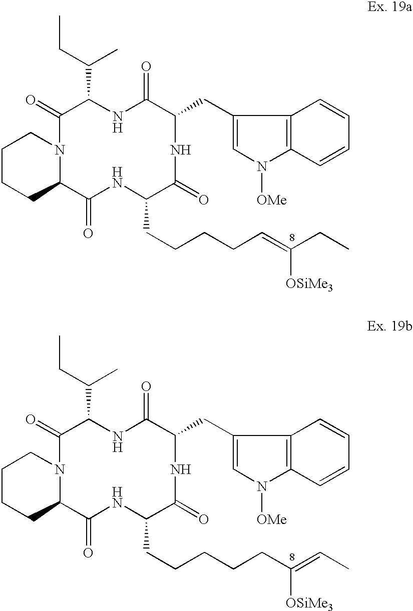

Examples 19a and 19b were prepared by the following procedure. 50 mg apicidin was heated in 5 mL THF at 50° C. until the resulting solution became homogenous. The solution was then cooled to −78° C. and immediately 800 μL 0.5M potassium hexamethyldisilazane in toluene was added. After 5 min, 40L TMSCl as a solution in 1 mL THF was added. After 10 min at −78° C. the reaction was stopped by the addition of 5 mL saturated NaHCO₃. Next, the solution was extracted, first with EtOAc, followed by CH₂Cl₂ and dried with Na₂SO₄. The crude mixture of Example 19a and Example 19b was used with no further purification in the next reaction. The crude yield was 74 mg (145%). The mixture was characterized by ¹H NMR. TLC: R_{f}=0.52 (1:2 acetone:hexanes).

### EXAMPLES 20A AND 20B

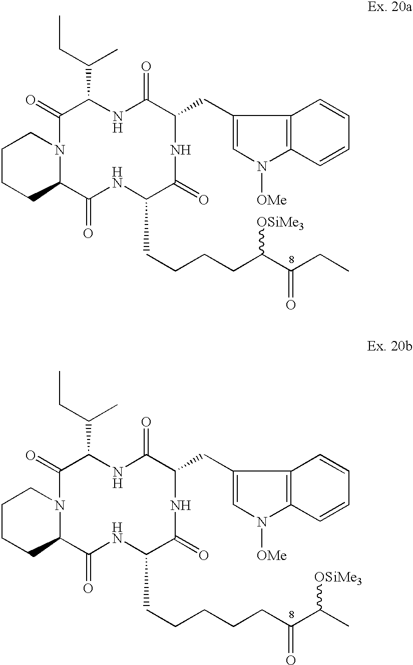

To form Examples 20a and 20b, 74 mg of the crude ˜1:1 mixture Example 19a, cyclo(N-O-methyl-L-Trp-L-Ile-D-Pip-L-2-amino-8 -trimethylsiloxy-7-ene- decanoyl), and Example 19b, cyclo(N-O-Methyl-L-Trp-L-Ile-D-Pip-L-2-amino-8-trimethylsiloxy-8-ene-decanoyl), was placed in 5 mL CH₂Cl₂ at RT to which was added 200 mg solid NaHCO₃. To this solution was added 20 mg 85% MCPBA. After 5 min, the reaction was quenched with 1:1 saturated Na₂S₂O₃:saturated NaHCO₃, extracted with CH₂Cl₂, and dried with Na₂SO₄. This yielded a 43 mg pure mixture of Example 20a, cyclo(N-O-methyl-L-Trp-L-Ile-D-Pip-L-2-amino-8-oxo-7-trimethylsiloxy-decanoyl), and Example 20b, cyclo(N-O-Methyl-L-Trp-L-Ile-D-Pip-L-2-amino-8-oxo-9-trimethylsiloxy-decanoyl) following flash chromatography on silica gel using 4:1 hexanes:acetone as eluant. The mixture was characterized by ¹H NMR. TLC: R_{f}=0.33 (1:2 acetone:hexanes).

### EXAMPLES 21A AND 21B

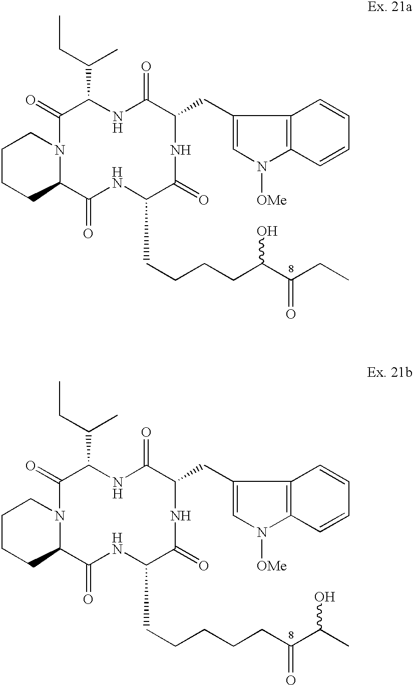

Example 21a and Example 21b were prepared by the following procedure. To 43 mg of a 1:1 mixture of Example 20a, cyclo(N-O-methyl-L-Trp-L-Ile-D-Pip-L-2-amino-8-oxo-7-trimethylsiloxy-decanoyl), and Example 20b, cyclo(N-O-Methyl-L-Trp-L-Ile-D-Pip-L-2-amino-8-oxo-9-trimethylsiloxy-decanoyl), in 4 mL THF at RT was added 120 μL 1M nBu₄NF in THF. After 20 min at RT, the solvent was evaporated under reduced pressure and the crude mixture purified by RP-HPLC without workup using 6:4 MeCN:H₂O. The resulting pure mixture of Examples 21a 1 and 21b was characterized by ¹H NMR and MS [m/z: 657.2 (M⁺+NH₄)]. TLC: R^{f}=0.14 (1:2 acetone:hexanes).

### EXAMPLES 22A AND 22B

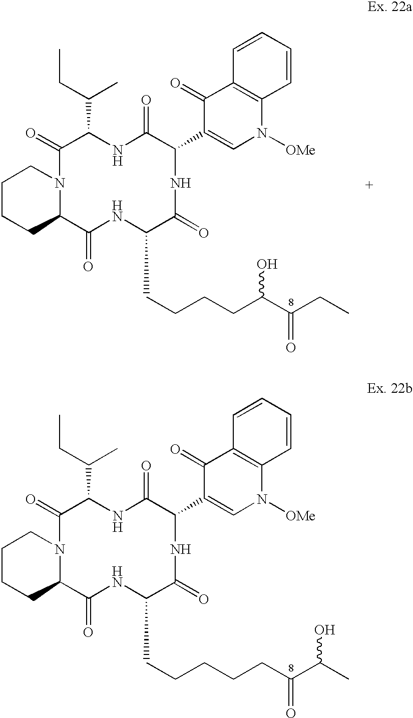

Following the general procedure of Examples 19-21, a 95% pure mixture of Examples 22a and 22b was prepared and characterized by ¹H NMR.

### EXAMPLES 23A AND 23B

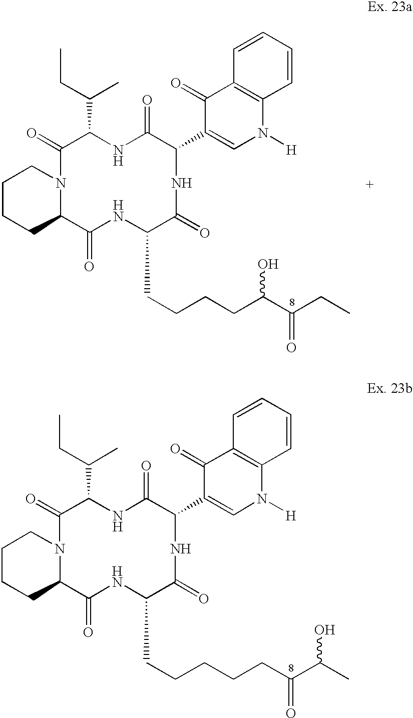

Following the general procedure of Examples 19-21, a 95% pure mixture of Example 23a and 23b was prepared and characterized by ¹H NMR.

### EXAMPLES 24A AND 24B

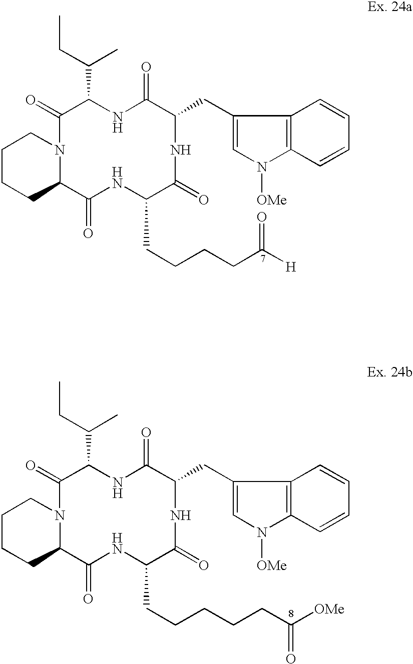

Examples 24a and 24b were prepared by adding 10 mL pyridine to 10 mg˜1:1 mixture of Examples 21a and 21b in 3 mL MeOH at 0° C., followed by the addition of 10 mg Pb(OAc)₄. After 10 min, the solution was quenched with 2 mL Na₂S₂O₃, diluted with about 2 mL brine, extracted with CH₂Cl₂ and dried with Na₂SO₄. Following preparative TLC on silica gel (500 μm plate) using 1:2 acetone:hexanes as eluant, separated pure products were obtained.

Example 24a, cyclo(N-O-Methyl-L-Trp-L-Ile-D-Pip-L-2-amino-7-oxo-heptanoyl) (5.5 mg) was characterized by ¹H NMR and MS [m/z: 582.2 (M⁺+1)]. TLC: R_{f}=0.16 (1:2 acetone:hexanes).

Example 24b, cyclo(N-O-Methyl-L-Trp-L-Ile-D-Pip-L-2-amino-7-carboxymethyl-heptanoyl) (6.5 mg) was characterized by ¹H NMR and MS [m/z: 626.3 (M⁺+1)]. TLC: R_{f}=0.23 (1:2 acetone:hexanes).

### EXAMPLES 25A-25D

Examples 25a-25d were prepared by following the general procedure of Example 24b. Starting with Examples 21a and 21b, and using an appropriate alcohol as solvent, the following derivatives were prepared and analyzed by NMR and mass spectroscopy:

TABLE 4

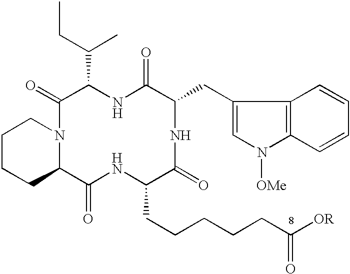

| Example | R Group | Mass Spec |
|---|---|---|
| 25a | Et | 640.5 (M⁺ + 1) |
| 25b | nPr | 654.4 (M⁺ + 1) |
| 25c | nBu | 668.3 (M⁺ + 1) |
| 25d | iPr | 654.4 (M⁺ + 1) |

### EXAMPLE 26

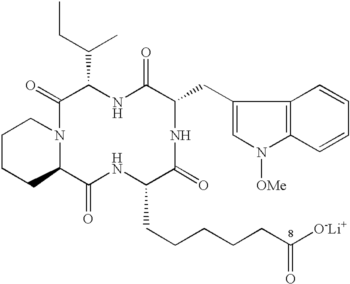

Example 26 was prepared by the following procedure. To 41 mg cyclo(N-O-methyl-L-Trp-L-Ile-D-Pip-L-2-amino-7-carbomethoxy-heptanoyl) in 4 mL 3:1:1 THF:MeOH:H₂O at 0° C. was added 100 μL 1M LiOH. The solution was stirred for 1 h and then additional 300 μL 1M LiOH was added. After 12 h, 33 mg pure Example 26 product was obtained following preparative RP-HPLC without workup using gradient elution (column equilibrated in 5:95 MeCN:H₂O, using 25:75 MeCN:H₂O for 40 min followed by a 20 min ramp to 100% MeCN, flow rate 10 mL/min). Example 26 was characterized by ¹H NMR and MS [m/z: 629.2 (M⁺+NH₄)]. HPLC: t_{R}=1.98 min 45:55 MeCN:H₂O, 1.5 mL/min, Zorbax™ RX-8).

### EXAMPLE 27

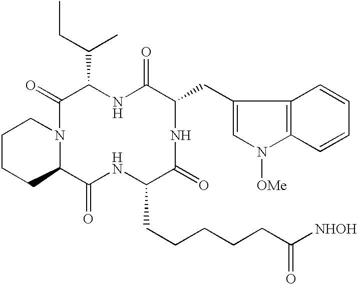

Example 27 was prepared by the following procedure. To 15 mg cyclo(N-O-methyl-L-Trp-L-Ile-D-Pip-L-2-amino-7-carboxy-heptanoyl), lithium salt in 3 mL DMF at RT was added 5.4 mg H₂NOSi(Me)₂tBu and 7 mg EDC.HCl. After 2 h at RT, 15 mg additional H₂NOSi(Me)₂tBu (15 mg) and 14 mg EDC.HCl were added and the solution allowed to stir overnight. The reaction was quenched by the addition of 5 drops glacial HOAc and 1 mL MeOH. The solution was poured into brine, extracted with CH₂Cl₂, and dried with Na₂SO₄. The crude product was chromatographed on silica gel using gradient elution (1:3:96 NH₄OH:MeOH:CHCl₃ to 1:4:95 NH₄OH:MeOH:CHCl₃, to 1:9:90 NH₄OH:MeOH:CHCl₃). To remove some contaminating EDU present in the chromatographed material, the product was dissolved in 2 mL CHCl₃ and 2 mL 10% aq. HOAc. After 5 min, the aqueous layer was decanted and the washing repeated twice more to yield 5.5 mg pure Example 27 product. The pure Example 27 stained positive (purple-orange) for a hydroxamic acid using Fe^{(III)}Cl₃ stain. The product was characterized by ¹H NMR and MS [m/z: 627.3 (M⁺+1)]. TLC: R_{f}=0.26 (then 1:9:90 NH₄OH:MeOH:CHCl₃).

### EXAMPLE 28

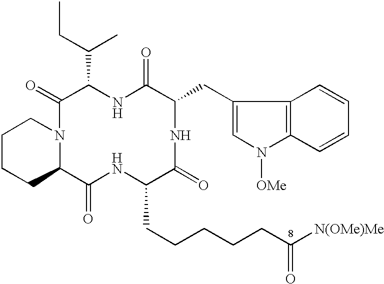

Example 28 was prepared by the following procedure. To 30 mg cyclo(N-O-methyl-L-Trp-L-Ile-D-Pip-L-2-amino-7-carboxy-heptanoyl) lithium salt in 1 mL DMF at RT was added 47 mg HCl.HN(OMe)Me, 2 mg DMAP, 7 mg HOBT (1-hydroxybenzotriazole hydrate) and 90 μL DIEA (Et₂NiPr) followed by 12 mg EDCl (1-(3-dimethylaminopropyl)-3-ethylcarbodiimide hydrochloride). After 36 h, the solution was poured into brine, acidified to pH˜4.0 with 2N HCl, extracted with CH₂Cl₂ and dried with Na₂SO₄. Following flash chromatography on silica gel using 1:3:96 NH₄OH:MeOH:CHCl₃ as eluant, 29.6 mg purified Example 28 was obtained and was characterized by ¹H NMR and MS [m/z: 655.3 (M⁺+1)]. TLC: R_{f}=0.39 (1:3:96 NH₄OH:MeOH:CHCl₃). HPLC: t_{R}=3.90 min (62:38 MeCN:H₂O, 1.5 mL/min, Zorbax™ RX-8).

### EXAMPLE 29

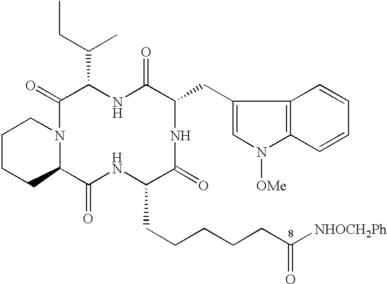

Example 29 was prepared by the following procedure. To 150 mg cyclo(N-O-methyl-L-Trp-L-Ile-D-Pip-L-2-amino-7-carboxy-heptanoyl) in 14 mL CH₂Cl₂ at 0° C. was added 78 mg HCl.H₂NOCH₂Ph, 0.13 mL DIEA, 33 mg HOBT, 2 mg DMAP, and 108 mg BOP. After 1 h at 0° C. and 12 h at RT, the solution was poured into saturated NaHCO₃, extracted with CH₂Cl₂ and dried with Na₂SO₄. Following preparative TLC on silica gel (5×1000 μm plates) using 5:95 MeOH:CHCl₃ as eluant, 137 mg pure Example 29 was obtained and was characterized by ¹H NMR. TLC: R_{f}=0.62 (5:95 MeOH:CHCl₃). HPLC: t_{R}=7.46 min (45:55 MeCN:H₂O, 1.5 mL/min, Zorbax™ RX-8).

### EXAMPLE 30

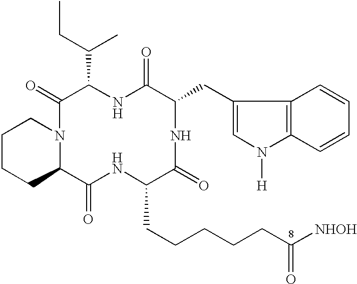

Example 30 was prepared by the following procedure. To 130 mg cyclo(N-O-methyl-L-Trp-L-Ile-D-Pip-L-2-amino-7-(N-benzyloxy-carboxamido)-heptanoyl) in 5 mL MeOH at RT was added 5% Pd/C and an H₂ atmosphere (balloon pressure) was established. After 12 h, 10 mg Pd(OH)₂ was added and the reaction continued for an additional 2 h. The catalyst was removed by filtration through Celite using MeOH as eluant and the solution concentrated under reduced pressure. Pure Example 30 product was obtained following RP-HPLC purification using gradient elution (5:95 MeCN:H₂O for 5 min then 55 min ramp to 50:50 MeCN:H₂O). The pure Example 30 was characterized by ¹H NMR and MS [m/z: 597.5 (M⁺+1)]. TLC: R_{f}=0.11 (1:9:90 NH₄OH:MeOH:CHCl₃). HPLC: t_{R}=10.65 min (2 min ramp from 5:95 MeCN:H₂O to 1:1 MeCN:H₂O, 1.0 mL/min, Zorbax™ RX-8).

### EXAMPLE 31

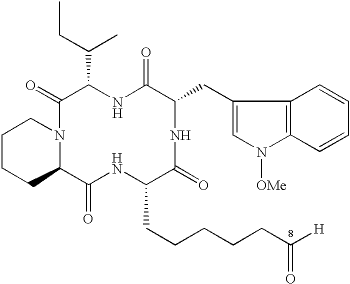

Example 31 was prepared by the following procedure. To 10 mg cyclo(N-O-methyl-L-Trp-L-Ile-D-Pip-L-2-amino-7-(N-O-methyl-N-methyl-carboxamido)-heptanoyl) in 2 mL THF at 0° C. was added 150 μL IM CH₂═CHMgBr in Et₂O. After 15 min at 0° C., the solution was cooled to −78° C. and quenched by addition of 1 mL saturated NH₄Cl. The solution was poured into brine and extracted with CH₂Cl₂ and dried with Na₂SO₄. The product was partially purified on a silica gel pipette plug using 1:2 acetone:hexanes as eluant. Following preparative TLC on silica gel (1×250 μm plate) using 1:3:96 NH₄OH:MeOH:CHCl₃ as eluant, 2.1 mg pure Example 31 was obtained and was characterized by ¹H NMR and MS [m/z: 596.3 (M⁺+1)]. TLC: R_{f}=0.57 (1:3:96 NH₄OH:MeOH:CHCl₃).

### EXAMPLE 32

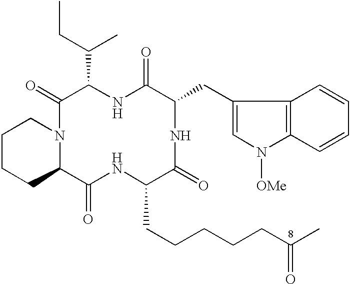

Example 32 was prepared by the following procedure. To 7 mg cyclo(N-O-methyl-L-Trp-L-Ile-D-Pip-L-2-amino-7-(N-methoxy-N-methyl-carboxamido)-heptanoyl) in 1 mL THF at 0° C. was added 55 μL 1M MeMgBr in Et₂O. After 10 min, an additional 55 μL 1M MeMgBr in Et₂O was added. The solution was poured into saturated NH₄Cl, extracted with CH₂Cl₂ and dried with Na₂SO₄. 4.3 mg pure Example 32 product was obtained following preparative TLC on silica gel (1×500 μm plate) using 4:6 acetone:hexanes as eluant. The pure Example 32 was characterized by ¹H NMR and MS [m/z: 610.3 (M⁺+1)]. TLC: R_{f}=0.22 (1:2 acetone:hexanes). HPLC: t_{R}=4.51 min (1:1 MeCN:H₂O, 1.5 mL/min, Zorbax™ RX-8).

### EXAMPLES 33A-33C

Following the general procedure illustrated in Example 26-34, the following derivatives were prepared:

TABLE 5

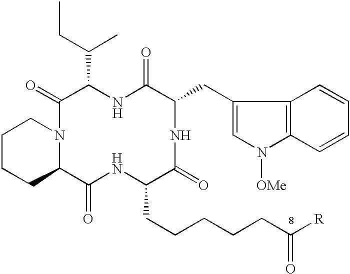

| Example | R Group | Mass Spec |
|---|---|---|
| 32 | Me | 610.3 (M⁺ + 1) |
| 33a | nPr | 638.5 (M⁺ + 1) |
| 33b | iPr | 638.5 (M⁺ + 1) |
| 33c | Ph | 672.5 (M⁺ + 1) |

### EXAMPLE 34

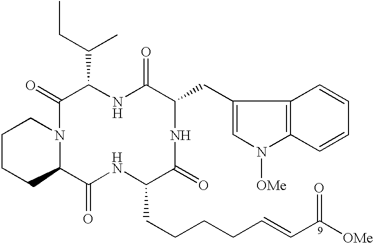

Example 34 was prepared by the following procedure. To 25 mg cyclo(N-O-methyl-L-Trp-L-Ile-D-Pip-L-2-amino-7-oxo-heptanoyl), 11 mg anhydrous LiCl, and 21 mL (MeO)₂P(O)CH₂CO₂Me in 2.5 mL MeCN at RT, was added 42 mL DIEA. After 2 h the solution was poured into saturated NaHCO₃, extracted with CH₂Cl₂ and dried with Na₂SO₄. Pure Example 34 product was obtained following flash chromatography on silica gel using 1:2 acetone:hexanes as eluant. The pure Example 34 was characterized by ¹H NMR and MS [m/z: 638.2 (M⁺+1)]. TLC: R_{f}=0.38 (1:2 acetone:hexanes). HPLC: t_{R}=5.09 min, (1:1 MeCN:H₂O, 1.5 mL/min, Zorbax™ RX-8).

### EXAMPLE 35

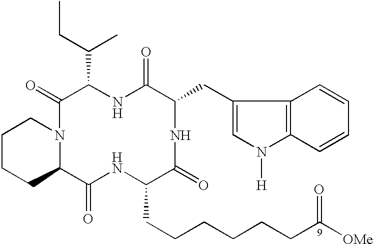

Example 35 was prepared by the following procedure. To 35 mg cyclo(N-O-methyl-L-Trp-L-Ile-D-Pip-L-2-amino-7,8-dehydro-8-carbomethoxy-octanoyl) in 4 mL 1:1 THF:MeOH was added 20 mg Pd(OH)₂ and an H₂ atmosphere (balloon pressure) was established. After 12 h, the catalyst was filtered off and 11.7 mg pure Example 35 product was obtained following flash chromatography on silica gel using 1:2 acetone:hexanes as eluant. The pure Example 35 was characterized by ¹H NMR. TLC: R_{f}=0.21 (1:2 acetone:hexanes). HPLC: t_{R}=3.84 min (55:45 MeCN:H₂O, 1.5 mL/min, Zorbax™ RX-8).

### EXAMPLE 36

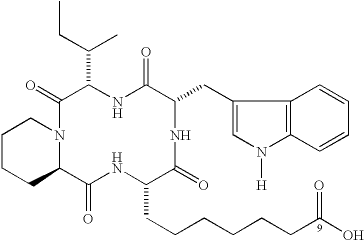

Example 36 was prepared by the following procedure. To 10.6 mg cyclo(N-O-methyl-L-Trp-L-Ile-D-Pip-L-2-amino-8-carbomethoxy-octanoyl) in 1 mL 3:1:1 THF:MeOH:H₂O at 0° C. was added 15 mL 1M LiOH. The solution was stirred for 1 h at 0° C., 6 h at RT, 3 days at 4° C. and then an additional 30 mL 1M LiOH was added. After 8 h longer, the solvents were removed using a vigorous stream of N₂ and pure Example 36 product was obtained by purification without workup using preparative RP-HPLC (gradient elution using 2:8 MeCN:H₂O for 10 min followed by a 60 min ramp to 100% MeCN). Pure product was characterized by ¹H NMR and MS [m/z: 596.3 (M⁺+1)]. HPLC: t_{R}=2.89 min (3:7 MeCN:H₂O, 1.5 mL/min, Zorbax™ RX-8).

### EXAMPLE 37

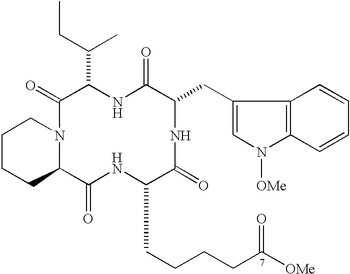

Example 37 was prepared by the following procedure. To 25 mg cyclo(N-O-methyl-L-Trp-L-Ile-D-Pip-L-2-amino-7-oxo-heptanoyl) in 1.25 mL DMF at RT was added 0.25 mL MeOH followed by 67.5 mg PDC. The solution was stirred for 3.5 h and then filtered through 1″ silica gel, with 0.5″ Celite on top of it, using MeOH as eluant. The solvents were removed under reduced pressure. Pure 9 mg Example 37 product was obtained following preparative TLC on silica gel (2×1000 μm plates) using 5:95 MeOH:CHCl₃ as eluant. The pure Example 37 was characterized by ¹H NMR and MS [m/z: 612.3 (M⁺+1)]. TLC: R_{f}=0.24 (1:2 acetone:hexanes). HPLC: t_{R}=9.41 min (45:55 MeCN:H₂O, 1.5 mL/min, Zorbax™ RX-8).

### EXAMPLE 38

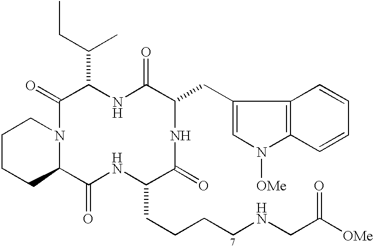

Example 38 was prepared by following the general procedure of Example 15, and Scheme III, utilizing methyl glycinate in place of ethylamine, and was characterized by ¹H NMR and MS [m/z: 655.0 (M⁺+1)].

### EXAMPLE 39

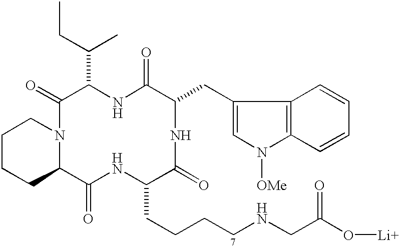

Example 39 was prepared by following the general procedure of Example 36 and starting with the methyl ester of Example 38, and was characterized by ¹H NMR and MS [m/z: 641.4 (M⁺+1)].

### EXAMPLE 40

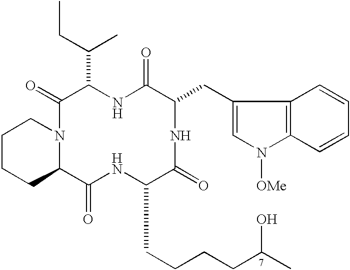

Example 40 was prepared by following the general procedure of Example 7, utilizing Example 32 as the starting material, and was characterized by ¹H NMR and MS [m/z: 598.3 (M⁺+1)].

### EXAMPLE 41

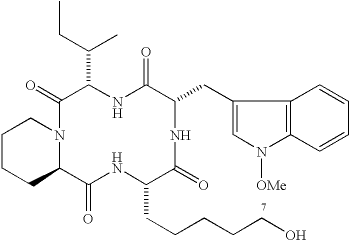

Example 41 was prepared by following the general procedure of Example 7 to convert the C7-aldehyde of Example 23 and was characterized by ¹H NMR and MS [m/z: 584.2 (M⁺+1)].

### EXAMPLE 42

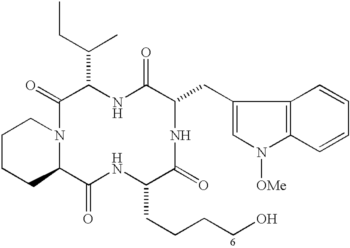

Example 42 was prepared by the following two methods.

Method A Following the general procedure of Example 7, the C₆-aldehyde of Example 58a was converted into Example 42 by adding 2.1 mg NaBH₄ to 64 mg cyclo(N-O-methyl-L-Trp-L-Ile-D-Pip-L-2-amino-6-oxo-hexanoyl) in 1 mL 1:1 THF:EtOH at 0° C. After 1 h, the resulting solution was poured into saturated NH₄Cl, extracted exhaustively with CH₂Cl₂ and 3:7 iPrOH:CHCl₃ (1×). The organic layer was dried with Na₂SO₄. Pure Example 42 was obtained following PTLC on silica gel (1×500 μm plate) using 1:1 acetone:hexanes as eluant. Example 42 was characterized by ¹H NMR and MS [m/z: 570 (M⁺+1)].

Method B

7.3 mg of a ˜1:1 mixture of 6,7- and 9,10-enones of apicidin, Example 55a and 55b, was placed in 1 mL CH₂Cl₂ at −78° C. Ozone was bubbled through the solution until a blue color persisted. A vigorous stream of nitrogen was then used to remove the excess ozone. To this solution was added 3.6 mg NaBH₄ in 120 μL 1:1 EtOH:H₂O, the cooling bath was removed and the solution was aged overnight. The solution was poured into saturated NH₄Cl(aq), extracted with CH₂Cl₂ and dried with Na₂SO₄. Pure Example 42 was obtained following PTLC purification on silica gel (1×500 μm plate) using 1:1 acetone:hexanes as eluant.

### EXAMPLE 43

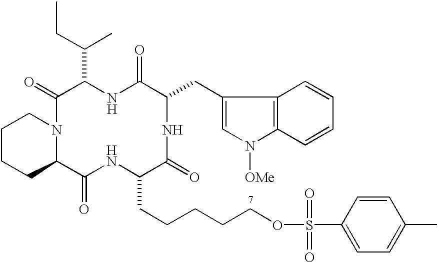

Example 43 was prepared by the following procedure. To 32 mg Example 41, cyclo(N-O-methyl-L-Trp-L-Ile-D-Pip-L-2-amino-7-hydroxy-heptanoyl), in 2.5 mL CH₂Cl₂ at 0° C., was added 27 μL DIEA, a catalytic amount of DMAP, and 36 mg toluene sulfonic anhydride. After 1 h at 0° C. and 12 h at RT, the solution was poured into saturated NaHCO₃, extracted with CH₂Cl₂ and dried with Na₂SO₄. Following preparative TLC on silica gel (2×1000 μm plates) using 1:3:96 NH₄OH:MeOH:CHCl₃ as eluant. 20 mg pure Example 43 product was obtained and was characterized by ¹H NMR and MS [m/z: 755.5 (M⁺NH₄)]. TLC: R_{f}=0.58 (1:3:96 NH₄OH:MeOH:CHCl₃).

### EXAMPLE 44

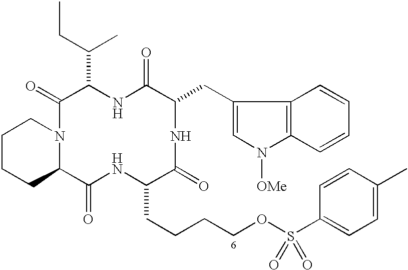

Example 44 was prepared from Example 42 cyclo(N-O-methyl-L-Trp-L-Ile-D-Pip-L-2-amino-6-hydroxy-hexanoyl) by following the general procedure of Example 43, and was characterized by ¹H NMR and MS [m/z: (M⁺+NH₄)].

### EXAMPLE 45

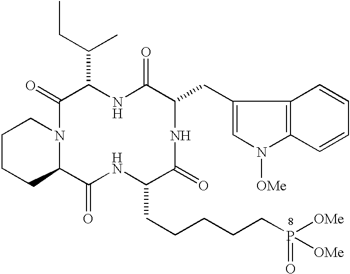

Example 45 was prepared by the following procedure. To 9 μL (MeO)₂P(O)H in 350 μL THF was added 2.5 mg 95% NaH at RT via syringe and the solution heated to reflux for 20 min. The solution was then cooled to RT and 25 mg cyclo(N-O-methyl-L-Trp-L-Ile-D-Pip-L-2-amino-7-(para-toluenesulfonyl)-heptanoyl) was added as a solution in 350 μL THF, heated to reflux for 2 h, cooled to RT and stirred for 12 h. The solution was poured into saturated NaHCO₃, extracted CH₂Cl₂ and dried with Na₂SO₄. Pure 4.1 mg Example 45 product was obtained following PTLC (1×1000 μm plate) on silica gel using 1:3:96 NH₄OH:MeOH:CHCl₃ as eluant. The pure product was characterized by ¹H NMR and MS [m/z: 676 (M⁺+1)].

### EXAMPLE 46

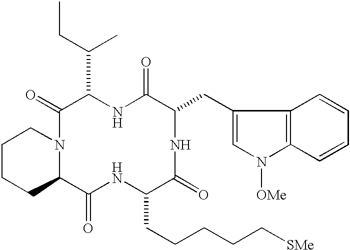

Example 46 was prepared by the following procedure. To 5 mg cyclo(N-O-methyl-L-Trp-L-Ile-D-Pip-L-2-amino-7-(para-toluenesulfonyl)-heptanoyl) in 1 mL DMF at RT was added 5 mg NaSMe. After 2 h, the solution was poured into brine, extracted with CH₂Cl₂ and dried with Na₂SO₄. The pure Example 46 product was obtained following preparative TLC on silica gel (1×500 μm plate) using 1:2 acetone:hexanes as eluant. The pure product was characterized by ¹H NMR and MS [m/z: 614.5 (M⁺+1)]. TLC: R_{f}=0.33 (1:2 acetone:hexanes).

### EXAMPLE 47

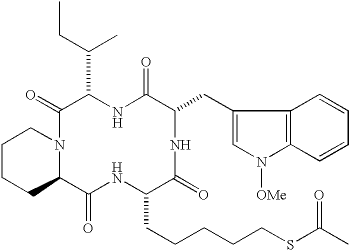

Example 47 was prepared by the following procedure. To 5 mg cyclo(N-O-methyl-L-Trp-L-Ile-D-Pip-L-2-amino-7-(para-toluenesulfonyl)-heptanoyl) in 1 mL DMF at RT was added 5 mg NaSAc. After 2 h, the solution was poured into brine, extracted with CH₂Cl₂ and dried with Na₂SO₄. Pure Example 47 product was obtained following preparative TLC on silica gel (1×500 μm plate) using 1:2 acetone:hexanes as eluant. The pure product was characterized by ¹H NMR and MS [m/z: 642.5 (M⁺+1)]. TLC: R_{f}=0.22 (1:2 acetone:hexanes).

### EXAMPLE 48

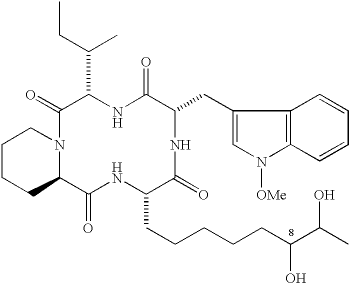

Example 48 was prepared by starting with Example 22b and following the general procedure described for Example 7. Example 22b's C8 ketone group was converted to a hydroxyl to form Example 48, which was characterized by ¹H NMR.

### EXAMPLE 49

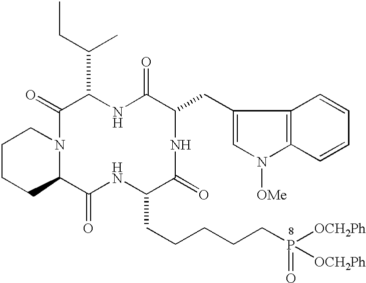

Example 49 was prepared by the following procedure. A solution of 63 μL dibenzyl phosphonate in 1 mL THF was added via syringe to 7 mg 95% NaH and the solution heated to reflux for 20 min. The mixture was cooled to RT and 70 mg cyclo(N-O-methyl-L-Trp-L-Ile-D-Pip-L-2-amino-7-(para-toluenesulfonyl)-octanoyl) was added as a solution in 1 mL THF. The resultant white, heterogeneous solution was heated to reflux for 2 h followed by 12 h at RT. The solution was added to water, extracted with CH₂Cl₂ and dried with Na₂SO₄. Pure 26 mg Example 49 was obtained following PTLC on silica gel (1×1500 μm plate) using 1:3:96 NH₄OH:MeOH:CHCl₃ as eluant. The product was characterized by ¹H NMR and MS [m/z: 828 (M⁺+1)].

### EXAMPLE 50

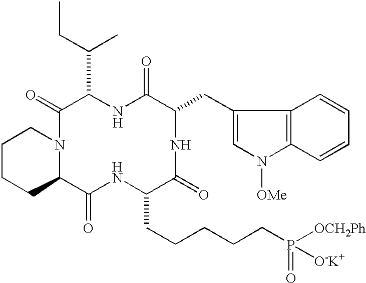

Example 50 was prepared by the following procedure. To 11 mg cyclo(N-O-methyl-L-Trp-L-Ile-D-Pip-L-2-amino-7-dibenzylphosphono-octanoyl), in 2 mL iPrOH containing 44 μL H₂O and 1.3 mg KHCO₃ at RT was added 1 mg 10% Pd/C. An atmosphere of H₂ was established (balloon pressure). After 12 h, the catalyst was removed by filtration through Celite using 1:1 MeOH:H₂O as eluant. The solution was concentrated in vacuo and the residue was washed with CHCl₃ followed by EtOAc. The remaining glassy material was lyophilized from water to yield 3 mg product. The product was characterized by ¹H NMR and MS [m/z: 738 (M⁺+1)].

### EXAMPLE 51

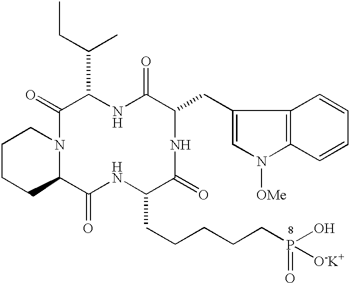

Example 51 was prepared by the following procedure. To 2 mg cyclo(N-O-methyl-L-Trp-L-Ile-D-Pip-L-2-amino-7-dibenzylphosphono-octanoyl) in 0.35 mL iPrOH was added 8 μL water, 0.25 mg KHCO₃ and 0.5 mg 10% Pd/C and a balloon atmosphere of hydrogen was established. After 7 h at RT, the catalyst was removed via filtration through Celite and washed with water. 3 mg of pure Example 51 product was characterized by ¹H NMR and MS [m/z: 648 (M⁺+1)].

### EXAMPLES 52A AND 52B

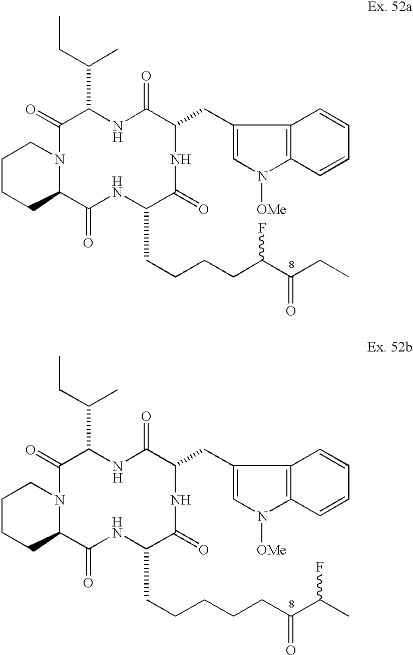

Examples 52a and 52b were prepared by the following procedure. To 3 mg˜1:1 cyclo(N-O-methyl-L-Trp-L-Ile-D-Pip-L-2-amino-7-hydroxy-8-oxo-decanoyl) and cyclo(N-O-methyl-L-Trp-L-Ile-D-Pip-L-2-amino-9- hydroxy-8-oxo-decanoyl) 3 mg) in 0.25 mL CH₂Cl₂ at −78° C. was added powdered, activated 4 Å sieves followed by 1.5 μL Et₂NSF₃. The solution was warmed to −10° C. over 1 h and then quenched by the addition of saturated NaHCO₃. The solution was extracted with CH₂Cl₂ and dried with Na₂SO₄. A pure mixture of approximately 1:1 Example 52a and 52b was obtained following PTLC on silica gel (1×500 μm plate) using 1:3:96 NH₄OH:MeOH:CHCl₃ as eluant. 2.5 mg of the mixture were characterized by ¹H NMR and MS [m/z: 641 (M⁺+1)].

### EXAMPLES 53A AND 53B

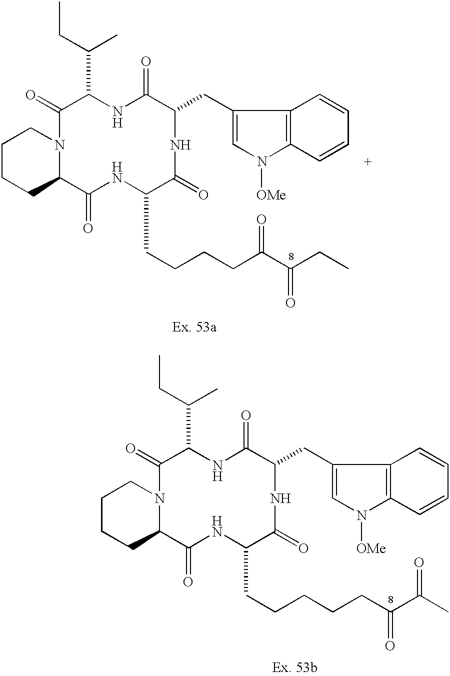

Examples 53a and 53b were prepared by the following procedure. To decanoyl), Examples 20a and 20b, at RT was added powdered, activated 4A sieves followed by 3 mg N-methylmorpholine-N-oxide and 0.3 mg TPAP. After 1 h, the mixture was diluted with CH₂Cl₂ and filtered through Celite using CH₂Cl₂ as eluant. The filtrate was extracted with 10% NaHSO₃(aq), washed with water, and dried with Na₂SO₄. Pure products were obtained following PTLC (1×500 μm plate) separation using 1:3:96 NH₄OH:MeOH:CHCl₃ as eluant. 3.5 mg of each pure Example 53a and 53b were characterized by ¹H NMR and MS [m/z: 637 (M⁺+1)].

### EXAMPLES 54A AND 54B

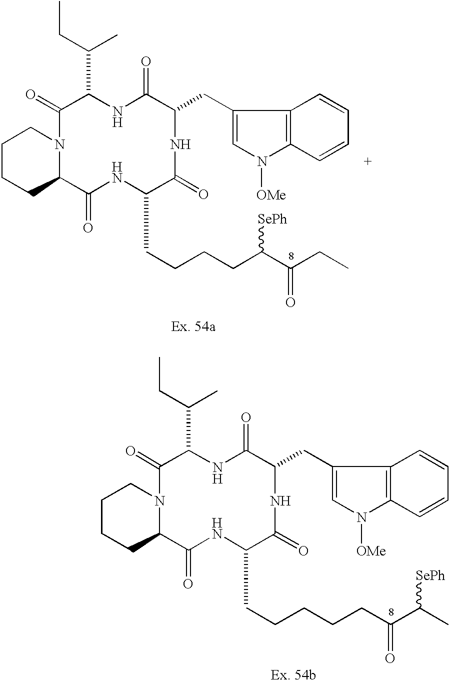

Examples 54a and 54b were prepared by the following procedure. To 2 g apicidin in 32 mL THF at 0° C. was added 14 mL 0.5M potassium hexamethyldisilazane solution in toluene. The solution was aged at 0° C. for 30 min. Next, 25.g solid PhSeCl was added and the solution was warmed to RT for 2 h. The reaction was quenched by the addition of saturated NaHCO₃(aq), was extracted with CH₂Cl₂, dried with Na₂S₄ and filtered through 8×14 cm plug of silica gel using gradient elution (hexanes to 1:1 EtOAc:hexanes to 1:1 acetone:hexanes). The product was used in the preparation of Examples 55a and 55b with no further purification. The 2.3 g mixture thus obtained was characterized by ¹H NMR and MS [m/z: 780.3 (Mg⁺+1)]. TLC: R_{f}=0.60(1:3:96 NH₄OH:MeOH:CHCl₃).

### EXAMPLES 55A AND 55B

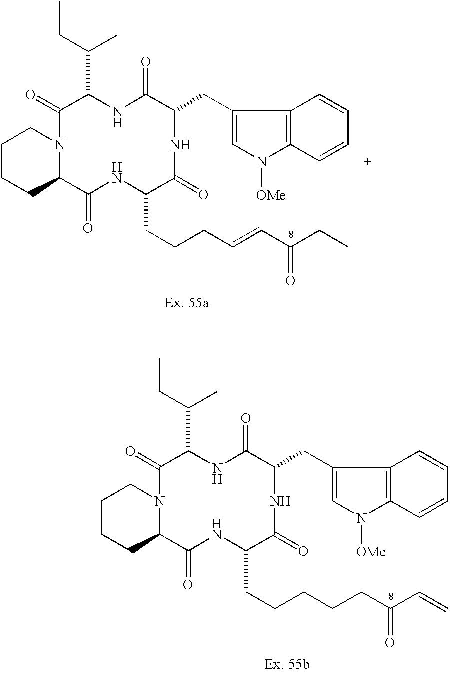

Examples 55a and 55b were prepared by the following procedure. To 2.2 g˜1:1 cyclo(N-O-methyl-L-Trp-L-Ile-D-Pip-L-2-amino-8-oxo-7-phenylselenyl-decanoyl) and cyclo(N-O-methyl-L-Trp-L-Ile-D-Pip-L-2-amino-8-oxo-9-phenylselenyl-decanoyl) in 40 mL THF at 0° C. was added 7.3 mL 30% H2O₂. The solution was warmed to 50° C. and after 10 min was cooled to 0° C., quenched with saturated Na₂S₂O₃, extracted with CH₂Cl₂ and dried with Na₂SO₄ Following purification on silica gel using 4:6 acetone:hexanes as eluant, a 230 mg pure mixture of Examples 55a and 55b was characterized by ¹H NMR and MS [m/z: 622.3 (M⁺+1)]. TLC:. R_{f}=0.38 (1:3:96 NH₄OH:MeOH:CHCl₃).

### EXAMPLES 56A AND 56B

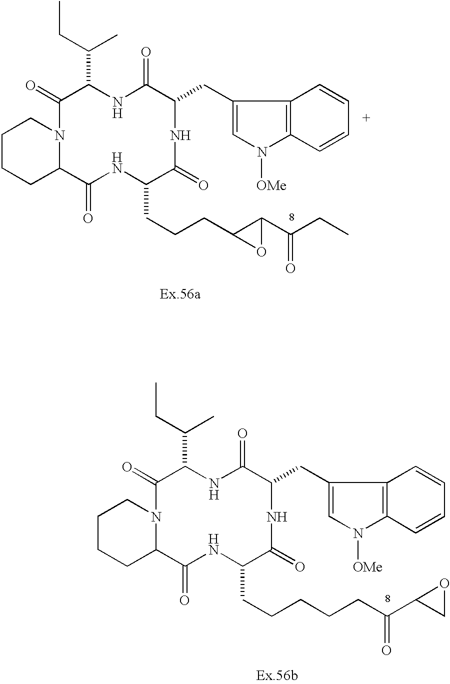

Examples 56a and 56b were prepared by the following procedure. To 5.6 mg˜1:1 cyclo(N-O-methyl-L-Trp-L-Ile-D-Pip-L-2-amino-8-oxo-6,7-dehydro-decanoyl) and cyclo(N-O-methyl-L-Trp-L-Ile-D-Pip-L-2-amino-8-oxo-9,10-dehydro-decanoyl) in 0.225 mL THF at RT was added 0.8 mL PhCH₂N(Me)₃ (40% solution in MeOH) followed by 1.5 mL 70% t-BuOOH(aq). After 4.5 h, EtOAc and minimal water were added and the aqueous phase thoroughly extracted with EtOAc. The organic layer was washed with cold 1N HCl (1×), quickly washed again with saturated NaHCO₃, and then dried with Na₂SO₄. Pure products were separated by PTLC (1×500 μm plate) using 4:6 acetone:hexanes as eluant. The pure examples 56a and 56b were characterized by ¹H NMR. The procedure yielded 1.4 mg cyclo(N-O-Methyl-L-Trp-L-Ile-D-Pip-L-2-amino-6,7-oxiranyl-decanoyl); MS [m/z: 638 (M⁺+1)]; TLC: R_{f}=0.4 (4:6 acetone:hexanes). The procedure yielded 2 mg cyclo(N-O-Methyl-L-Trp-L-Ile-D-Pip-L-2-amino-9,10-oxiranyl-decanoyl); MS [m/z: 638 (M⁺+1)]; TLC: R_{f}=0.3 (4:6 acetone:hexanes).

### EXAMPLES 57A AND 57B

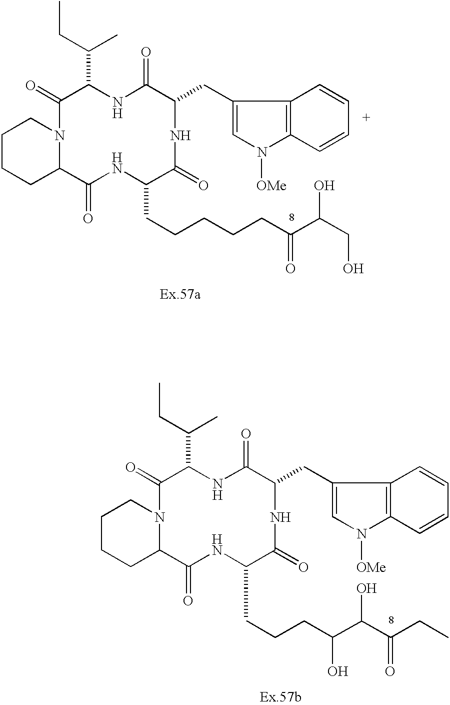

Example 57a and 57b were prepared by the following procedure. To 115 mg˜1:1 cyclo(N-O-methyl-L-Trp-L-Ile-D-Pip-L-2-amino-8-oxo-6,7-dehydro-decanoyl) and cyclo(N-O-methyl-L-Trp-L-Ile-D-Pip-L-2-amino-8-oxo-9,10-dehydro-decanoyl) in 4 mL 8:1 acetone:water at 0° C. was added 45 mg trimethylamine-N-oxide followed by 0.77 mL 0.024M OsO₄(aq). The solution was warmed to RT for 3 h and then aged at 4° C. for 12 h. The brown homogenous solution was quenched at 0° C. by the addition of 2 mL 10% NaHSO₃(aq). After 10 min, brine was added and the solution thoroughly extracted with 3:7 iPrOH:CHCl₃ (9×) and dried with Na₂SO₄. The solvent was removed in vacuo to yield 230 mg crude product (121 mg theoretical) which was used with no additional purification. A small aliquot of the regioisomeric diols were separated by PTLC on silica gel (1×1000 μm plate) using 1:1 acetone:hexanes as eluant and the products were characterized by ¹H NMR and MS. cyclo(N-O-methyl-L-Trp-L-Ile-D-Pip-L-2-amino-8-oxo-6,7-dihydroxy-decanoyl): MS [m/z: 656 (M⁺+1)]; TLC: R_{f}=0.5 (4:6 acetone:hexanes). cyclo(N-O-methyl-L-Trp-L-Ile-D-Pip-L-2-amino-8-oxo-9,10-dihydroxy-decanoyl): MS [m/z: 656 (M⁺+1)]; TLC: R_{f}=0.25 (4:6 acetone:hexanes).

### EXAMPLES 58A AND 58B

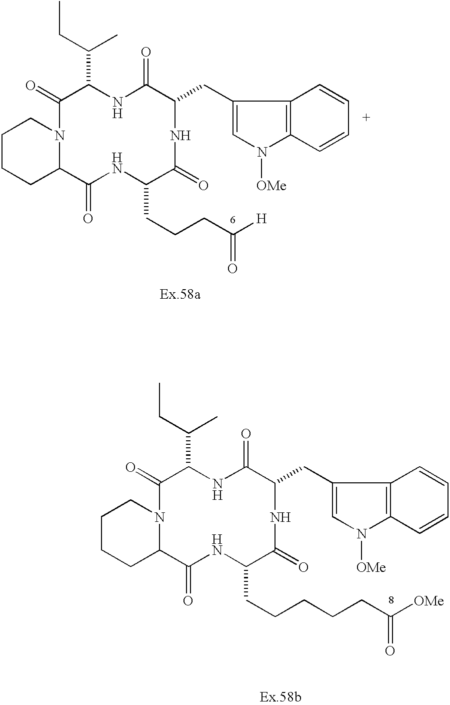

Examples 58a and 58b were prepared by the following procedure. To 121 mg˜1:1 cyclo(N-O-methyl-L-Trp-Ile-D-Pip-L-2-amino-8-oxo-6,7-dihydroxy-decanoyl) and cyclo(N-I-methyl-L-Trp-L-Ile-D-Pip-L-2-amino-8-oxo-9, 10-dihydroxy-decanoyl) in 6 mL MeOH at 0° C. was added 75 mL pyridine followed by 184 mg Pb(OAc)₄. After 40 min, the solution was poured into saturated Na₂S₂O₃, extracted with CH₂Cl₂ and dried with Na₂SO₄. Pure separated Examples 58a and 58b were obtained following PTLC (3×1500 μm plate) on silica gel using 1:3:96 NH₄OH:MeOH:CHCl₃ as eluant. The pure products were characterized by ¹H NMR and MS.

Example 58a, cyclo(N-O-Methyl-L-Trp-L-Ile-D-Pip-L-2-amino-6-oxo-hexanoyl): Yield: 30 mg. MS [m/z: 568 (M⁺+1)]; TLC: R_{f}=0.45 (1:1 acetone:hexanes).

Example 58b, cyclo(N-O-Methyl-L-Trp-L-Ile-D-Pip-L-2-amino-7-carboxymethyl-heptanoyl): Yield: 20 mg.

### EXAMPLE 59

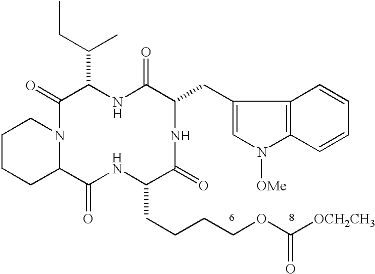

Example 59 was prepared by the following procedure. To 4 mg cyclo(N-O-methyl-L-Trp-L-Ile-D-Pip-L-2-amino-6-hydroxy-hexanoyl) in 0.14 mg CH₂Cl₂ and 1 mL pyridine at 0° C. was added 1 mL ethyl chloroformate. The solution was warmed to RT, aged for 3 h and the solvents removed in vacuo. 1.3 mg pure Example 59 was obtained following PTLC (1×500 μm plate) on silica gel using 4:6 acetone:hexanes as eluant. The pure product was characterized by ¹H NMR and MS [m/z: 659 (M⁺+NH₄)].

### EXAMPLE 60

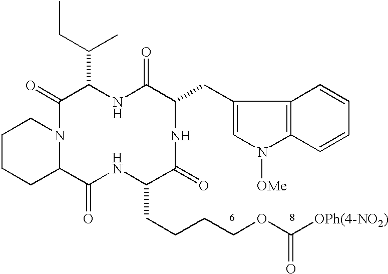

Example 60 was prepared by the following procedure. 7.5 mg of the Example 64 C₆-alcohol was placed in about 1 mL CH₂Cl₂ at 0° C. to which was added 3.2 mg (4-NO₂)PhOC(O)Cl followed by 1.3 μL pyridine. After 2 h at 0° C., the volatiles were removed under reduced pressure without workup and 9 mg pure Example 60 product was obtained following PTLC on silica gel (1×500 μm plate) using 1:1 acetone:hexanes as eluant. The pure Example 60 thus prepared was characterized by ¹H NMR and MS [m/z: 735 (M⁺+1)].

### EXAMPLE 61

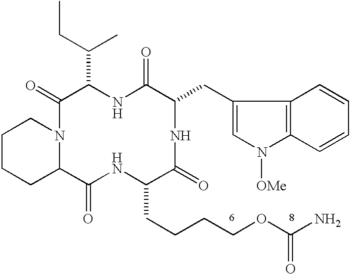

Example 61 was prepared by the following procedure. Anhydrous ammonia was bubbled into 2 mL dioxane at 0° C. to generate a ˜0.5 M solution. This solution was added to 6 mg solid cyclo(N-O-methyl-L-Trp-L-Ile-D-Pip-L-2-amino-6-para-nitrophenoxycarbonyloxy-hexanoyl) at 0° C. The ice bath was removed and the solution aged at RT for 2 h. The solution was concentrated under reduced pressure and 1.7 mg pure Example 61 was obtained following PTLC (1×500 μm plate) using 1:9:90 NH₄OH:MeOH:CHCl₃ as eluant, and was characterized by ¹H NMR and MS [m/z: 613 (M⁺+1)].

### EXAMPLE 62

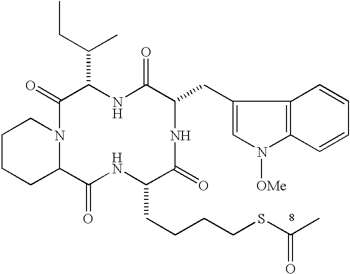

Example 62 was prepared by the following procedure. To 4 mg Ph₃P in 0.2 μL THF at 0° C. was added 2.4 mL DEAD (diethyl azodicarboxylate) and aged for 30 min. To this resulting solution was added about 4 mg solid cyclo(N-O-methyl-L-Trp-L-Ile-D-Pip-L-2-amino-6-hydroxy-hexanoyl) at 0° C. After 1 h at 0° C., the solution was warmed to RT for 1 h. Solvent was removed under reduced pressure. 2 mg of pure Example 62 product was obtained following PTLC (1×250 μm plate) using 1:1 acetone:hexanes as eluant and was characterized by ¹H NMR and MS [m/z: 628 (M⁺+1)].

### EXAMPLE 63

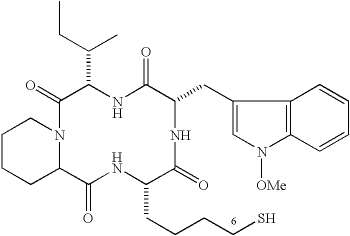

Example 63 was prepared by the following procedure. To 1.5 mg cyclo(N-O-methyl-L-Trp-L-Ile-D-Pip-L-2-amino-6-acetylthio-hexanoyl) in 0.2 mL MeOH at 0° C. was added 0.3 mL 25 wt % NaOMe in MeOH. After 5 h, water was added to quench the reaction. The solution was extracted with CH₂Cl₂ and dried with Na₂SO₄. 0.5 mg pure Example 63 product was obtained following PTLC (1×250 μm plate) using 1:1 acetone:hexanes as eluant. Example 63 was characterized by ¹H NMR and MS [m/z: 588 (M⁺+1)].

### EXAMPLE 64

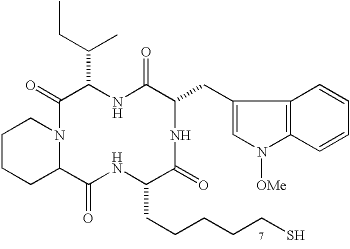

Following the general procedure of Example 63, the C7 thiol was prepared from the corresponding thioacetate of Example 47. Example 64 was characterized by ¹H NMR and MS [m/z: 599 (M⁺+1)].

### EXAMPLE 65

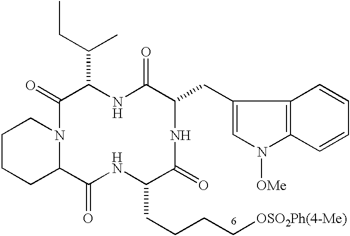

Example 65 was prepared by the following procedure. To 1.6 mg cyclo(N-O-methyl-L-Trp-L-Ile-D-Pip-L-2-amino-6-hydroxy-hexanoyl) in 0.28 mL CH₂Cl₂ at 0° C. was added 0.2 mg DMAP followed by 2 mg TsCl. After 16 h, the solution was aged at RT for 16 h. The solvent was removed under reduced pressure. Following PTLC (1×250 μm plate) using 1:3:96 NH₄OH:MeOH:CHCl₃ as eluant, 0.3 mg pure Example 65 was obtained. The pure product was characterized by ¹H NMR and MS [m/z: 724 (M⁺+1)].

### EXAMPLE 66

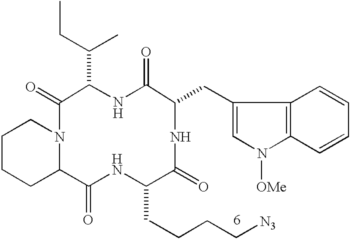

Example 66 was prepared by the following procedure. To 4 mg cyclo(N-O-methyl-L-Trp-L-Ile-D-Pip-L-2-amino-6-hydroxy-hexanoyl) in 0.35 mL CH₂Cl₂ at 0° C. was added i) 3.7 mg PPh₃, ii) 1 mg imidazole and iii) 3.2 mg Zn(N3)₂.(pyridine)₂ followed by iv) 2.2 μL DEAD. The solution was warmed to RT for 12 h. Following PTLC (1×500 μm plate) using 1:3:96 NH₄OH:MeOH:CHCl₃ as eluant, 2 mg pure Example 66 was obtained. Example 66 was characterized by ¹H NMR and MS [m/z: 595 (M⁺+1)].

### EXAMPLE 67

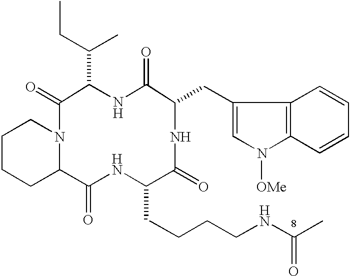

Example 67 was prepared by the following procedure. To 1 mg cyclo(N-O-methyl-L-Trp-L-Ile-D-Pip-L-2-amino-6-azido-hexanoyl) at 0° C. in 0.1 mL THF was added 0.1 mL thiolacetic acid. After 1 h, the solution was warmed to RT for 1 h. The solvents were then removed with a vigorous stream of nitrogen. The residue was dissolved in 0.2 mL neat thiolacetic acid, aged for 4 h and then concentrated with a vigorous stream of nitrogen. Pure Example 67 product was obtained following PTLC (1×250 μm plate) using 1:3:96 NH₄OH:MeOH:CHCl₃ as eluant. The pure product (0.7 mg) was characterized by ¹H NMR and MS [m/z: 611 (M⁺+1)].

### EXAMPLE 68

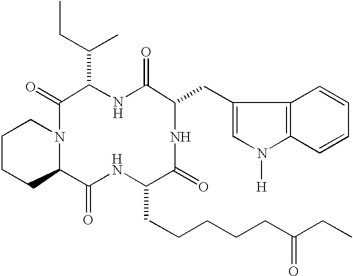

Example 68 was prepared by the following procedure. 40 mg Pd(OH)₂ was added to 500 mg apicidin in 40 mL 1:1 THF:MeOH. An H₂ atmosphere was established (balloon pressure). After 12 h, the palladium catalyst was removed by filtration through Celite using MeOH as eluant. Following flash chromatography on silica gel using 4:6 acetone:hexanes as eluant, 467 mg pure Example 68 product was obtained and was characterized by ¹H NMR. TLC: R_{f}=0.18 (1:2 acetone:hexanes). HPLC: t_{R}=7.54 min (1:1 MeCN:H₂O, 1.5 mL/min, Zorbax™ RX-8).

### EXAMPLE 69

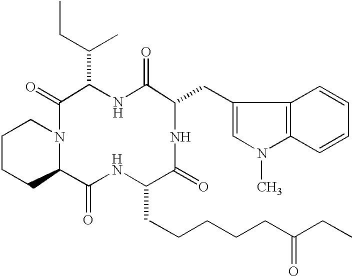

Example 69 was prepared by the following methods:

Method C

To 30 mg N-desmethoxy apicidin in 500 μL DMF at RT was added 4 drops MeI followed by the addition of 11 mg tBuOK. The solution was stirred for 2 h at RT, 12 h at 4° C. and then for another 4 h at RT. The solution was then heated at 60° C. for 1.5 h and cooled back to RT. An additional 20 mg tBuOK was added and the solution stirred for 1 h. The solution was then poured into 3 mL of 1:2 saturated NaHCO₃:saturated brine, extracted with CH₂Cl₂ and dried with Na₂SO₄. Following preparative TLC on silica gel (2×1500 μm plates) using 1:2 acetone:hexanes as eluant, 19 mg pure Example 69 was obtained and was characterized by ¹H NMR and MS [m/z: 625.3 (M⁺+NH₄)]. TLC: R_{f}=0.31(1:2 acetone:hexanes). HPLC: t_{R}=3.90 min (62:38 MeCN:H₂O, 1.5 mL/min, Zorbax™ RX-8).

Method D

1.3 mg 60% NaH was added to 20 mg N-desmethoxy apicidin in 0.35 mL DMF at RT). After 30 min, 4 μL MeI was added and the solution stirred for 10 h. The solution was then poured into saturated NH₄Cl, extracted with CH₂Cl₂ and dried with Na₂SO₄. Following preparative TLC on silica gel (1×500 μm plate) using 1:1 acetone:hexanes as eluant, and further purification by preparative RP-HPLC using a linear gradient (1:1 to 1:0 MeCN:H₂O), 5 mg pure Example 69 was obtained which was characterized by ¹H NMR and MS [m/z: 608.5 (M⁺+1)].

### EXAMPLE 70

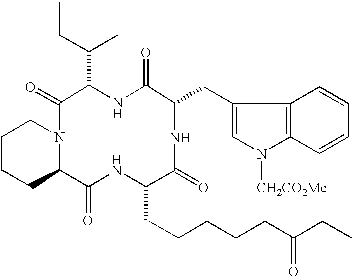

Example 70 was prepared by the following procedure. At RT, 467 mg N-Desmethoxy apicidin was placed in 16 mL DMF to which was added 63 mg 60% NaH. After 10 min, 206 μL BrCH₂CO₂Me and 871 mg nBu₄NI were added and the solution heated to 80° C. After 15 min, the solution was poured into water, extracted with CH₂Cl₂ and dried with Na₂SO₄. Following flash chromatography on silica gel using 1:1 acetone:hexanes as eluant, 401 mg pure Example 70 was obtained which was characterized by ¹H NMR and MS [m/z: 666 (M⁺+1)]. TLC: R_{f}=0.46(1:1 acetone:hexanes). HPLC: t_{R}=7.21 min 1:1 MeCN:H₂O, 1.0 mL/min, Zorbax™ RX-8).

### EXAMPLE 71

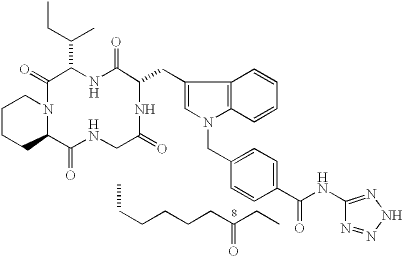

Example 71 was prepared by the following procedure. At 0° C., 0.65 mg HOBT, 1.6 mg NaHCO₃, 0.5 mg 5-aminotetrazole and 1 mg EDCI was added to 3.5 mg N-desmethoxy-N-(para-carboxyphenylmethyl) apicidin in DMF. After 12 h, the solution was poured into saturated NHCl₄, extracted with CH₂Cl₂ and dried with Na₂SO₄ Following RP-HPLC using gradient elution (4:6 to 1:0 MeCN:water), 1.6 mg pure Example 71 was obtained which was characterized by ¹H NMR and MS [m/z: 795 (M⁺+1)].

### EXAMPLE 72

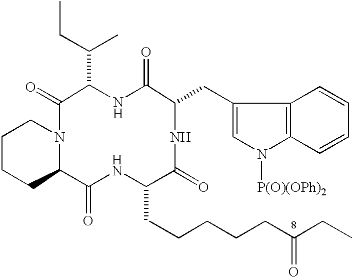

Example 72 was prepared by the following procedure. At RT, 3.4 mg 60% NaH was added to 50 mg N-desmethoxy apicidin in 0.2 mL DMF and 0.2 mL HMPA. After gas evolution ceased, 35 μL (PhO)₂P(O)Cl was added. After 24 h, the solution was poured into water, extracted with EtOAc and dried with Na₂SO₄. Following preparative chromatotron TLC (1000 μm plate) using 1:2 acetone:hexanes as eluant, 16 mg pure Example 72 was obtained which was characterized by ¹H NMR and MS [m/z: 826 (M⁺+1)].

### EXAMPLE 73

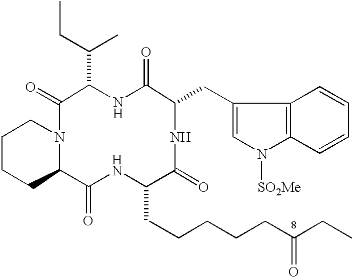

Example 73 was prepared by the following procedure. At RT, 7 mL Et₃N, and 1 mg DMAP was added to 10 mg N-desmethoxy apicidin in 0.17 mL CH₂C₂. Then 3.9 μL MeSO₂Cl was added. After 20 h, the solution was poured into water, extracted with EtOAc and dried with Na₂SO₄. Following preparative RP-HPLC using a linear gradient (4:6 to 1:0 MeCN:H₂O), 0.6 mg pure Example 73 was obtained (R_{f}=0.4, 4:6 acetone:hexanes) which was characterized by ¹H NMR and MS [m/z: 672 (M⁺+1)].

### EXAMPLES 74A-74J

Following the general procedure of Examples 69-72, utilizing an appropriate electrophile (R-X) readily determined by one in the art, the following compounds were prepared:

TABLE 6

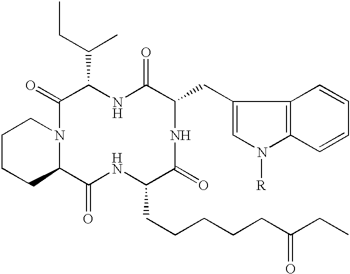

| Example | R Group | Mass Spec |
|---|---|---|
| 69 | Me | 608.5 (M⁺ + 1) |
| 70 | CH₂CO₂Me | 666 (M⁺ + 1) |
| 71 | CH₂Ph[4-C(O)NH(5-tetrazolyl)] | 795 (M⁺ + 1) |
| 72 | P(O)(OPh)₂ | 826 (M⁺ + 1) |
| 73 | SO₂Me | 672 (M⁺ + 1) |
| 74a | Et | 639.4 (M⁺ + NH₄) |
| 74b | nPr | 653.3 (M⁺ + NH₄) |
| 74c | CH₂CO₂tBu | 708 (M⁺ + 1) |
| 74d | CH₂CH₂OSi(tBu)Me₂ | 752 (M⁺ + 1) |
| 74e | CH₂Ph(4-CO₂Me) | 742 (M⁺ + 1) |
| 74f | C(O)Ph(4-Oac) | 756 (M⁺ + 1) |
| 74g | C(O)Ph | 698 (M⁺ + 1) |
| 74h | CO₂Ph(4-N0₂) | 759 (M⁺ + 1) |
| 74i | CO₂CH₂Ph | 728 (M⁺ + 1) |
| 74j | SO₂Ph(4-Me) | 748 (M⁺ + 1) |
| 75 | CO₂CH₂CH₂NMe₂ | 709 (M⁺ + 1) |

### EXAMPLE 75

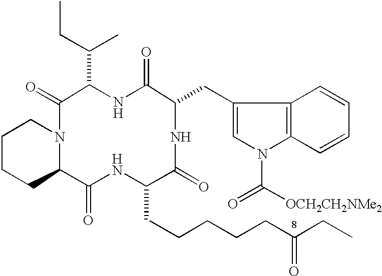

Example 75 was prepared by the following procedure. At RT, 0.1 mL pyridine was added to 9 mg N-desmethoxy-N-(para-aminophenoxycarbonyl) apicidin in 0.22 mL DMF, followed by the addition of 22 μL HOCH₂CH₂NMe₂. After 15 h, the solution was poured into saturated NaHCO₃, extracted with CH₂Cl₂ and dried with Na₂SO₄. Following preparative chromatotron TLC (1000 μm plate) using 1:2 acetone:hexanes as eluant, pure Example 75 was obtained which was characterized by ¹H NMR and MS [m/z: 709 (M⁺+1)].

### EXAMPLE 76

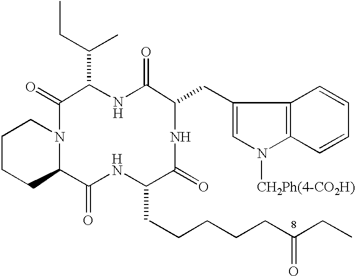

Example 76 was prepared by the following procedure. At 0° C., 7.8 μL 1N LiOH was added to 3.8 mg N-desmethoxy-N-(para-carboxymethylphenylmethyl) apicidin in 0.13 mL of a 3:1:1 mixture of THF:MeOH:H₂O. After 2 h at 0° C. and 17 h at RT, the volatiles were then removed with a vigorous stream of nitrogen. The aqueous layer was then extracted with EtOAc and the aqueous layer acidified to pH˜4 with 2N HCl. The aqueous layer was further extracted with 5 aliquots of a 3:7 mixture of iPrOH:CHCl₃ and finally dried with Na₂SO₄. Following RP-HPLC using a linear gradient (2:8 to 1:0 MeCN:H₂O), 2.5 mg pure Example 76 was obtained which was characterized by ¹H NMR and MS [m/z: 728 (M⁺+1)].

### EXAMPLE 77

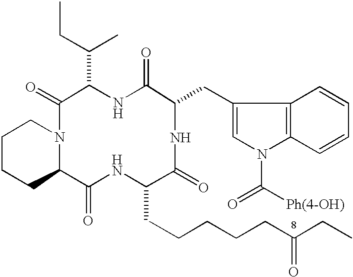

Example 77 was prepared by the following procedure. At −10° C., 6.5 μM LiOH was added to a solution of 3.3 mg N-desmethoxy-N-(para-acetoxyphenylcarbonyl) apicidin in 0.11 mL of a 3:1:1 mixture of THF:MeOH:H₂O. After 1 h, the volatiles were removed with nitrogen. Then, about 2 mL each of water and EtOAc was added. The resulting solution was carefully neutralized to pH˜7 with 2N HCl. The solution was extracted with EtOAc and dried with Na₂SO₄. Following PTLC (1×500 μm plate) using 6:4 acetone:hexanes as eluant, 1.7 mg pure Example 77 was obtained which was characterized by ¹H NMR and MS [m/z: 714 (M⁺+1)].

### EXAMPLE 78

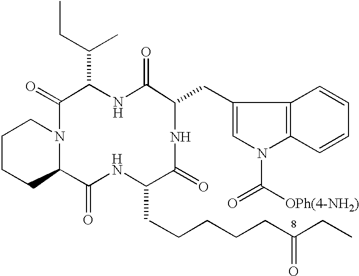

Example 78 was prepared by the following procedure. At RT, 0.5 mg 10% Pd/C catalyst was added to 2 mg N-desmethoxy-N-(para-nitrophenoxycarbonyl)-apicidin in 0.2 mL CH₂Cl₂ and an atmosphere of hydrogen established (balloon pressure). After 6.5 h, the catalyst was removed by filtration through Celite using 1:1 MeOH:CH₂Cl₂ as eluant. Without any further purification, the resulting 1.8 mg Example 78 was characterized by ¹H NMR and MS [m/z: 729 (M⁺+1)].

### EXAMPLE 79

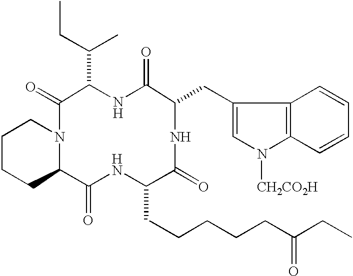

Example 79 was prepared by the following procedure. At 0° C., 200 μL 1M LiOH was added to 89 mg N-desmethoxy-N-carbomethoxymethyl apicidin in 3.5 mL of a 1:1:1 mixture of THF:MeOH:H₂O. After 45 min at 0° C., the slightly cloudy solution was warmed to RT and became homogenous. After an additional 20 min, the MeOH and THF were removed using a vigorous stream of N₂. Then, 2 mL Ethyl acetate was added to the solution and removed to dispose of residual organic soluble material. The solution was acidified to pH˜4.0 using 2N HCl, 3 mL brine was added to the aqueous layer, and then extracted with a 1:4 mixture of iPrOH:CHCl₃. The organic layer was dried with Na₂SO₄ to yield 51 mg pure Example 79, which was characterized by ¹H NMR and MS [m/z: 652.5 (M⁺+1)]. HPLC: t_{R}=1.21 min (1:1 MeCN:H₂O, 1.5 mL/min, Zorbax™ RX-8).

### EXAMPLE 80

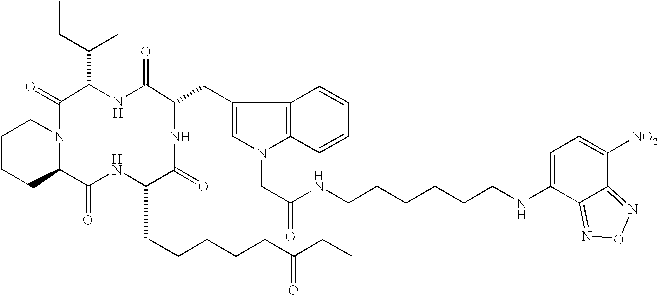

Example 80 was prepared by the following procedure. At RT, 2.6 mL TEA was added to 6 mg N-desmethoxy-N-(6-amino-hexylaminocarbonylmethyl)-apicidin in 1 mL CH₂Cl₂. Next, 4 mg NBD-Cl was added and the vial was wrapped with foil. After 3 h at RT, pure Example 80 was obtained by flash chromatography on silica gel without workup using 1:1 hexanes:acetone as eluant. The pure product was characterized by ¹H NMR. TLC: R_{f}=0.19 (1:1 acetone:hexanes).

### EXAMPLE 81

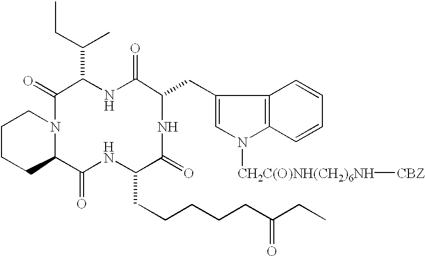

Example 81 was prepared by the following procedure. At 0° C., 19 mg EDCI was added to 50 mg N-desmethoxy-N-carboxymethyl apicidin, 29 mg CBZ-HN(CH₂)₆NH₂, 10 mg HOBT and 19 μL DIEA in 5 mL CH₂Cl₂. After 15 min at 0° C. and 1 h at RT, 3 mg DMAP was added. After an additional 2 hours, the CH₂Cl₂ was removed using a vigorous stream of N₂ and 2 mL DMF was added. After 2 h, the solution was poured into 20 μL 2:1 H₂O:brine, acidified to pH˜3.0 with 2N HCl and extracted with 5 15 mL aliquots of CH₂Cl₂. The organic layer was dried with Na₂SO₄. Without further purification, 54 mg pure Example 81 was obtained which was characterized by ¹H NMR and MS [m/z: 884.6 (M⁺+1)]. TLC: R_{f}=0.72 (1:9:90 NH₄OH:MeOH:CHCl₃). HPLC: t_{R}=5.38 min (6:4 MeCN:H₂O, 1.5 mL/min, Zorbax™ RX-8).

### EXAMPLE 82

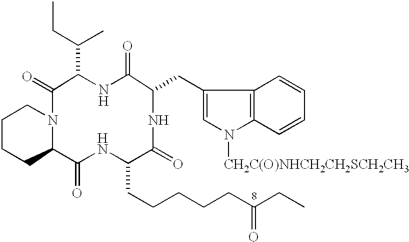

Example 82 was prepared by the following procedure. At 0° C., 1.2 mg HOBT was added to 5.7 mg N-desmethoxy-N-carboxymethyl apicidin in 0.1 mL DMF, 2.9 mg NaHCO₃, and 1.2 mg EtSCH₂CH₂NH₂.HCl. This was followed by the addition of 1.8 mg EDCI. The solution was warmed to RT and aged for 16 h. The aged solution was poured into saturated NaHCO₃, extracted with EtOAc and dried with Na₂SO₄. Following RP-HPLC using gradient elution (4:6 to 1:0 MeCN:H₂O), 3.3 mg pure Example 82 was obtained which was characterized by ¹H NMR and MS [m/z: 739 (M⁺+1)].

### EXAMPLE 83

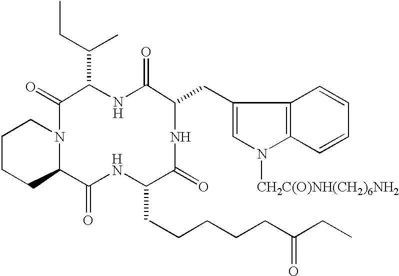

Example 83 was prepared by the following procedure. At RT, 10 mg 5% Pd/C catalyst was added to 54 mg N-desmethoxy-N-[6-(benzyloxycarbonylamino)-hexylaminocarbonylmethyl]-apicidin in 3 mL DMF and a H₂ atmosphere (balloon pressure) was established. After 2 h, an additional 40 mg 5% Pd/C catalyst was added and the solution stirred overnight. The catalyst was then filtered off and the solvents were removed under reduced pressure. Following flash chromatography on silica gel using gradient elution (using first neat CHCl₃, then three subsequent elutions of 1:3:96, then 1:4:95 and then 1:9:90 NH₄OH:MeOH:CHCl₃ as eluant), pure Example 83 was obtained which was characterized by ¹H NMR and MS [m/z: 750.4 (M⁺+1)]. TLC: R_{f}=0.12 (1:9:90 NH₄OH:MeOH:CHCl₃).

### EXAMPLE 84

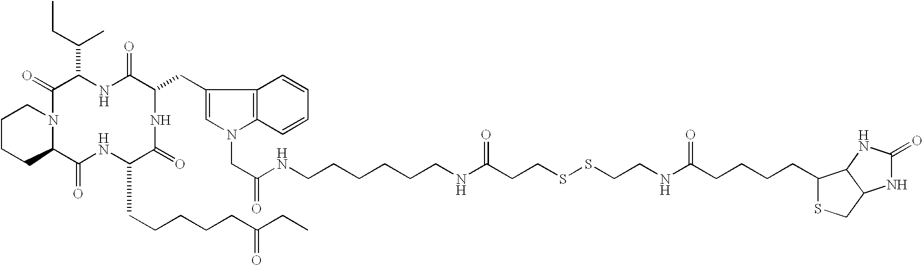

Example 84 was prepared by the following procedure. At RT, 3.2 mg NHS-SS-Biotin was added to 4 mg N-desmethoxy-N-(6-aminohexylaminocarbonylmethyl)-apicidin in 0.5 mL CH₂Cl₂ followed by 2 μL DIEA. The solution was stirred for 1 h at RT, followed by 12 h at 4° C. and 2 h at RT. Additional 3.2 mg NHS-SS-Biotin and 2 μL DIEA were added followed by 100 μL DMF. After an additional 1 hour, the solution was loaded directly onto a silica gel pipette column using gradient elution (1:3:96 to 1:9:90 NH₄OH:MeOH:CHCl₃ as eluant) to yield 4 mg pure Example 84 which was characterized by ¹H NMR. TLC: R_{f}=0.26 (1:9:90 NH₄OH:MeOH:CHCl₃).

### EXAMPLE 85

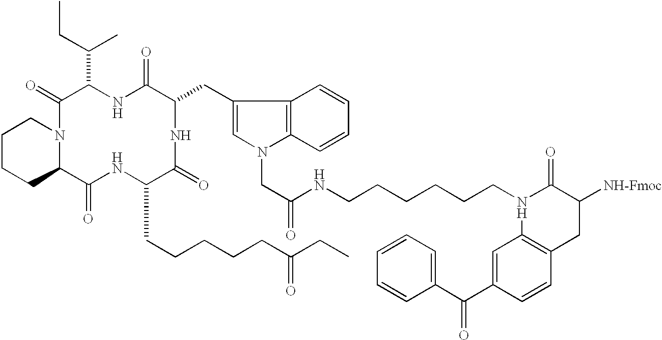

Example 85 was prepared by the following procedure. At RT, 0.5 mg HOBT, 2.6 mg Fmoc-Phe(4-Bz)-OH (Fmoc=9-fluorenylmethyl oxycarbonyl) and 1 mg EDCI was added to 2 mg N-desmethoxy-N-(6-aminohexylaminocarbonylmethyl)-apicidin in 0.5 mL CH₂Cl₂. Then, 3 μL DIEA was added. After 2 h at RT, the crude was purified without workup on a pipette flash column with silica gel using gradient elution (1:1 acetone:hexanes followed by 5:95 MeOH:CHCl₃). The partially purified Example 85 was characterized by ¹H NMR. TLC: R_{f}=0.26 (1:9:90 NH₄OH:MeOH:CHCl₃). TLC: R_{f}=0.53 (5:95 MeOH:CHCl₃).

### EXAMPLE 86

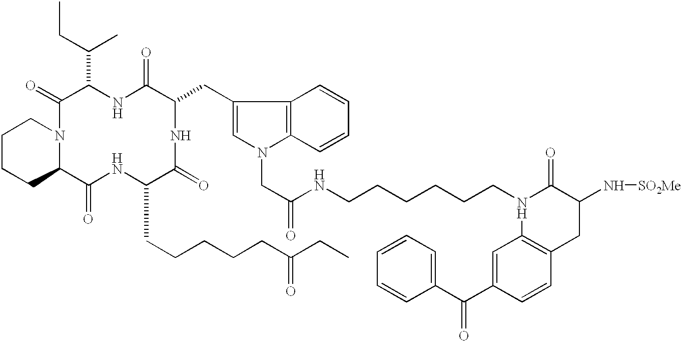

Example 86 was prepared by the following procedure. At RT, 0.2 mL piperidine was added to 15 mg of the Fmoc-protected Example 85 compound in 2 mL CH₂Cl₂. After 3 h at RT, the volatiles were removed under reduced pressure to produce Example 86. This material was used with no additional purification in Example 87.

### EXAMPLE 87

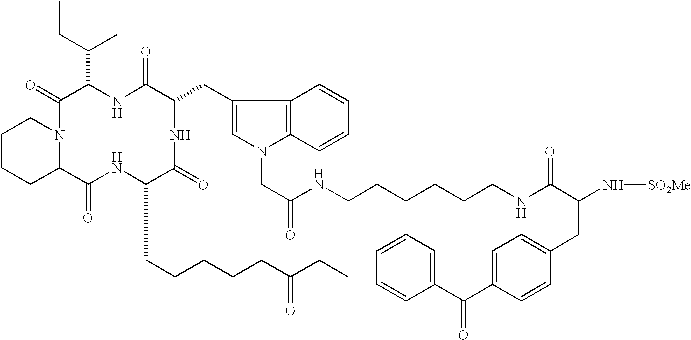

Example 87 was prepared by adding 5 μL Et₃N to 2 mg of the crude product of Example 86 in 0.2 mL CH₂Cl₂ at 0° C. followed by 2 μL MeSO₂Cl. After 30 min, the reaction was quenched by the addition of 3 drops of a 1:9:90 mixture of NH₄OH:MeOH:CHCl₃. Following flash chromatography on silica gel using 1:3:96 NH₄OH:MeOH:CHCl₃ as eluant, pure Example 87 was obtained without workup which was characterized by ¹H NMR.

### EXAMPLE 88

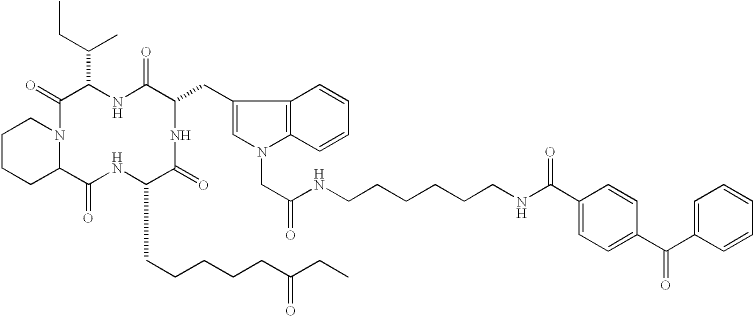

Example 88 was prepared by the following procedure. First, 6 mg HOBT, 10 mg (4-Bz)PhCO₂H, 23 μL DIEA, and 19.6 mg BOP were added to 250 μL CH₂Cl₂ at RT to generate (4-Bz)PhCO(OBT). Then, 20 μL of freshly prepared (4-Bz)PhCO(OBT) solution was added to 1 mg N-desmethoxy-N-(6-aminohexylaminocarbonylmethyl)-apicidin in 200 μL CH₂Cl₂ in a vial. The vial was wrapped in foil and allowed to stir at RT overnight. Partially purified product was obtained following preparative TLC on silica gel (1×250 μm plate) using 1:9:90 NH₄OH:MeOH:CHCl₃ as eluant. Following preparative TLC on silica gel (1×250 μm plate) using 1:3:96 NH₄OH:MeOH:CHCl₃ as eluant, pure Example 88 was obtained which was characterized by ¹H NMR. TLC: R_{f}=0.27 (1:3:96 NH₄OH:MeOH:CHCl₃).

### EXAMPLE 89

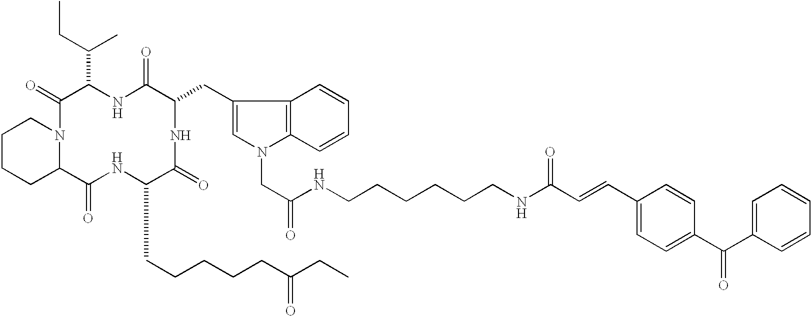

Example 89 was prepared by the following procedure. At RT, 3 mg HOBT, 6 μL Et₃N, and 4.1 mg (4-Bz)PhCH═CHCO₂H was added to 9 mg N-desmethoxy-N-(6-aminohexylaminocarbonylmethyl)-apicidin in 1 mL CH₂Cl₂ followed by 13 mg BOP. After 4 h, the crude was purified without workup by flash chromatography on silica gel using 1:3:96 NH₄OH:MeOH:CHCl₃. This yielded 13.4 mg pure Example 89, which was characterized by ¹H NMR. TLC: R_{f}=0.29 (1:3:96 NH₄OH:MeOH:CHCl₃). HPLC: T_{R}=4.90 min (7:3 MeCN:H₂O, 1.5 mL/min, Zorbax™ RX-8).

### EXAMPLE 90

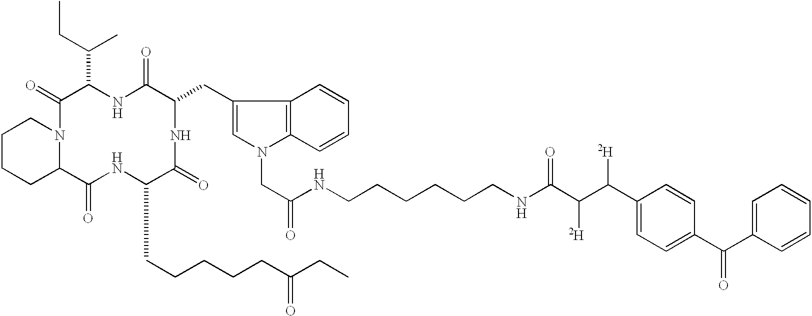

Example 90 was prepared by the following procedure. At RT, 3 mg 5% Pd/C catalyst was added to 4 mg Example 89 in 1:1 MeOH:CH₂Cl₂ and a deuterium gas atmosphere was established (balloon pressure). After 1 h, the solution was purified on a silica gel pipette column using 1:9:90 NH₄OH:MeOH:CHCl₃ as eluant to yield 2.9 mg pure Example 90, which was characterized by ¹H NMR. TLC: R_{f}=0.34 (1:3:96 NH₄OH:MeOH:CHCl₃). HPLC: t_{R}=4.66 min (7:3 MeCN:H₂O, 1.5 mL/min, Zorbax™ RX-8).

### EXAMPLE 91

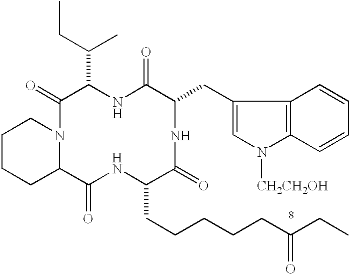

Example 91 was prepared by the following procedure. To 9 mg of the silyl ether Example 74d in 0.2 mL pyridine at 0° C. was added 0.2 mL HF.pyridine solution (prepared from 25 g HF.pyridine, 10 mL pyridine and 25 mL THF). After 1.5 h, the reaction was quenched by the addition of saturated NaHCO₃, extracted with CH₂Cl₂ and the combined organic layers were dried with Na₂SO₄. The 7.4 mg of alcohol thus obtained was used in Example 92 below with no additional purification and was characterized by ¹H NMR and MS [m/z: 638 (M⁺+1)].

### EXAMPLE 92

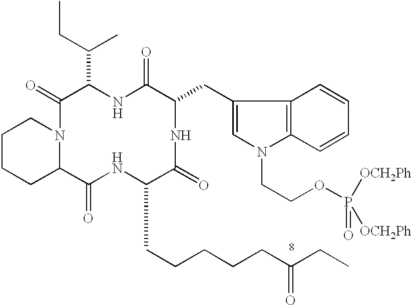

Example 92 was prepared by adding to 7.4 mg of the Example 91 alcohol in 4 mL CH₂Cl₂ at RT 422 mg 1,2,4-triazolyle followed by 610 μL (PhCH₂O)₂PNEt₂. After aging the solution for 3 h, the volatiles were removed in vacuo to form a yellow residue. Then, 7 mL THF was added to the yellow residue to form a solution, which was cooled to −40° C. To this solution was added 4.6 mL 30% H₂O₂ and warmed to RT. After aging for 30 min, the reaction was quenched by the addition of 10% Na₂S₂O₃(aq), diluted with saturated NaHCO₃(aq) and water, extracted with CH₂Cl₂ and dried with Na₂SO₄. Following chromatotron purification (1000 μm plate) using 1:2 acetone:hexanes as eluant, 255 mg pure Example 92 was obtained which was characterized by ¹H NMR and MS [m/z: 898 (M⁺+1)].

### EXAMPLE 93

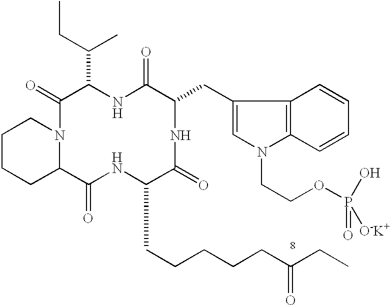

Example 93 was prepared by adding 27 mg KHCO₃ and 25 mg 10% Pd/C catalyst at RT to 245 mg of Example 92 in 40 mL iPrOH and 1 mL water. An atmosphere of hydrogen (balloon pressure) was established for 12 h. After the catalyst was removed by filtration through Celite using 1:1 MeOH:H₂O as eluant, the volatiles were removed under reduced pressure. No further purification was required and yielded 214 mg Example 93, which was characterized by ¹H NMR and MS [m/z: 718 (M⁺+1)].

### EXAMPLE 94

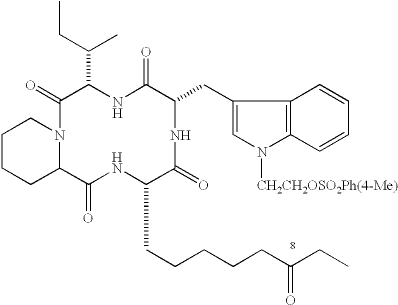

Example 94 was prepared by the following procedure. At 0° C., 2 mg DMAP was added to 20 mg apicidin alcohol in 2 mL CH₂Cl₂ followed by the addition of 26 mg Ts₂O. After 10 min the solution was warmed to RT for 3 h. Then, 10 mg TsCl was added and the solution aged for 16 h. The solvent was removed under reduced pressure and 1 mg pure Example 94 was obtained following centrifugal TLC (4:6 acetone:hexanes to 1:9:90 NH₄OH:MeOH:CHCl₃) as eluant. The product was characterized by ¹H NMR and MS [m/z: 792(M⁺+1)].

### EXAMPLE 95

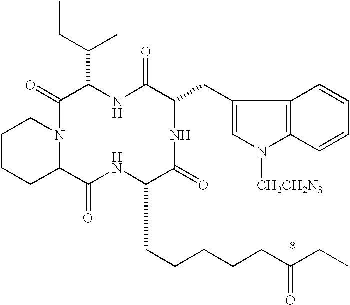

Example 95 was prepared by the following procedure. At 0° C., 247 mg Ph₃P and 217 mg Zn(N₃)₃.pyridine was added to 300 mg N-desmethoxy-N-(2-hydroxyethyl)-apicidin in 25 mL CH₂Cl₂, followed by the addition of 150 μL DEAD. The solution was then warmed to RT. After aging for 12 h, the volatiles were removed under reduced pressure. Following chromatotron TLC on silica gel (2 mm plate) using 1:3:96 NH₄OH:MeOH:CHCl₃ as eluant, 311 mg pure Example 95 (R_{f}=0.32, 1:9:90 NH₄OH:MeOH:CHCl₃) was obtained which was characterized by ¹H NMR and MS [m/z: 663 (M⁺+1)].

### EXAMPLE 96

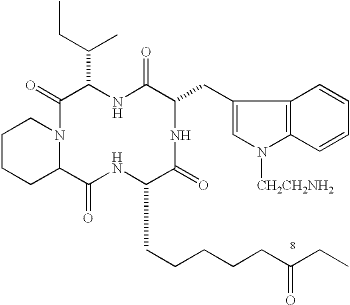

Example 96 was prepared by the following procedure. At RT, 60 mg 10% Pd/C catalyst was added to 311 mg N-desmethoxy-N-(2-azidoethyl) apicidin in CH₂Cl₂ and an atmosphere of hydrogen was established (balloon pressure). After 8 h, the catalyst was filtered through Celite using 3:7 iPrOH:CHCl₃ as eluant to yield the desired product. Following chromatotron PTLC (1×2000 μm plate) using 1:3:96 NH₄OH:MeOH:CHCl₃ as eluant, pure Example 96 (200 mg, R_{f}=0.21 (1:3:96 NH₄OH:MeOH:CHCl₃) was obtained which was characterized by ¹H NMR and MS [m/z: 637 (M⁺+1)].

### EXAMPLE 97

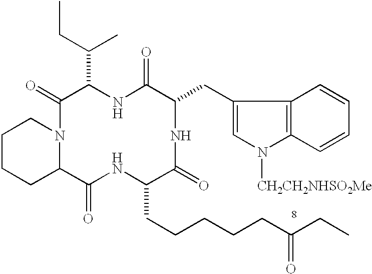

Example 97 was prepared by the following procedure. At 0° C., 9 μL Et₃N was added to 10 mg N-desmethoxy-N-(2-aminoethyl) apicidin in 0.5 mL CH₂Cl₂ followed by the addition of 3.6 μL MeSO2Cl. The solution was warmed to RT and stirred for 30 min. The solution was quenched by the addition of saturated NaHCO₃, extracted with CH₂Cl₂ and dried with Na₂SO₄. Following PTLC (1×250 μm plate) on silica gel using 1:1 acetone:hexanes as eluant, 9 mg pure Example 97 was obtained which was characterized by ¹H NMR and MS [m/z: 732.7 (M⁺+NH₄)]. TLC: R_{f}=0.26 (1:1 acetone:hexanes). HPLC: t_{R}=4.7 min (1:1 MeCN:H₂O, 1.5 ml/min, Zorbax™ RX-C8).

### EXAMPLE 98

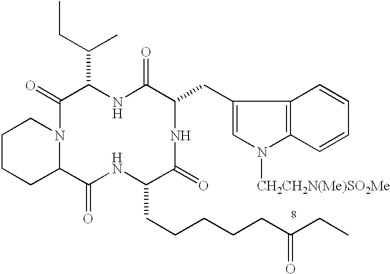

Example 98 was prepared by the following procedure. At RT, 7 μL NaN(TMS)₂ (IM in THF) was added to 4 mg N-desmethoxy-N-2-methanesulfonamidoethyl apicidin in 0.28 mL THF followed by the addition of 1.5 μL MeI. After 16 h, the solution was quenched by the addition of water, extracted with CH₂Cl₂ and dried with Na₂SO₄. Following PTLC (1×250 μm plate) on silica gel using 1:3:96 NH₄OH:MeOH:CHCl₃ as eluant, 2.2 mg pure Example 98 was obtained which was characterized by ¹H NMR and MS [m/z; 746.6 (M⁺+NH₄)]. TLC: R_{f}=0.42 (1:3:96 NH₄OH:MeOH:CHCl₃).

### EXAMPLE 99

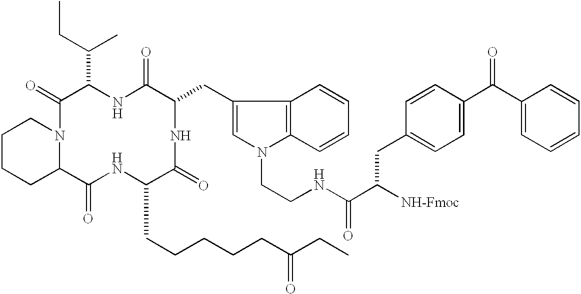

Example 99 was prepared by the following procedure. At RT, 5 mg HOBT, 7 μL TEA and 18.4 mg Fmoc-Phe(4-Bz)-OH was added to 16 mg N-desmethoxy-N-(2-aminoethyl)-apicidin in 1 mL CH₂Cl₂ followed by the addition of 16 mg BOP. After 3 h at RT, the solution was purified by flash chromatography on silica gel using 1:3:96 NH₄OH:MeOH:CHCl₃ as eluant to yield pure Example 99, which was characterized by ¹H NMR. TLC: R_{f}=0.50(1:3:96 NH₄OH:MeOH:CHCl₃).

### EXAMPLE 100

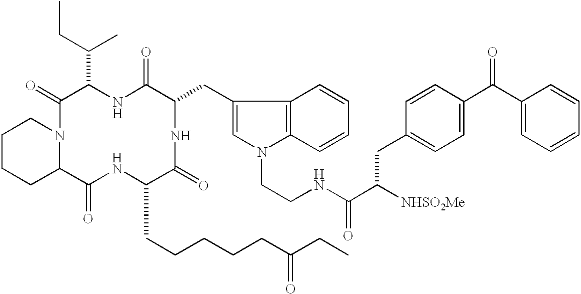

Example 100 was prepared by adding 50 μL piperidine to 15 mg of the Fmoc-protected amine of Example 99 at RT in 2 mL CH₂Cl₂. After 2 h at RT, the solution was concentrated under reduced pressure and lyophilized from dioxane to remove residual piperidine. The crude deprotected amine product was dissolved in 2 mL CH₂Cl₂ at 0° C. and 5.6 μg Et₃N was added followed by 62 μL MeSO₂Cl (0.26M in CH₂Cl₂). After 1 h, the reaction was quenched by the addition of saturated NaHCO₃(aq), extracted with CH₂Cl₂ and dried with Na₂SO₄. Following PTLC on silica gel (1×1000 μm plate) using 1:4:95 NH₄OH:MeOH:CHCl₃ as eluant, pure Example 100 was obtained which was characterized by ¹H NMR and MS [m/z: 1080 (M⁺+1)].

### EXAMPLE 101

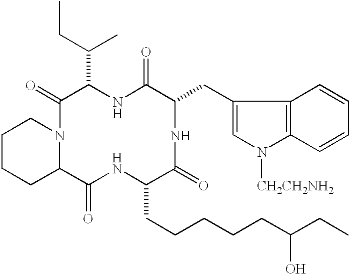

Example 101 was prepared by the following procedure. At RT, 8 mg NaBH₄ was added to 20 mg N-desmethoxy-N-(2-aminoethyl) apicidin in 2 mL MeOH. After 2 h at RT, acetone was added to the solution to quench the reaction and the solution was poured into saturated NaHCO₃, extracted with CH₂Cl₂ and dried with Na₂SO₄. Following flash chromatography on silica gel using 1:9:90 NH₄OH:MeOH:CHCl₃ as eluant, pure Example 101 was obtained which was characterized by ¹H NMR. TLC: R_{f}=0.28 (1:9:90 NH₄OH:MeOH:CHCl₃).

### EXAMPLE 102

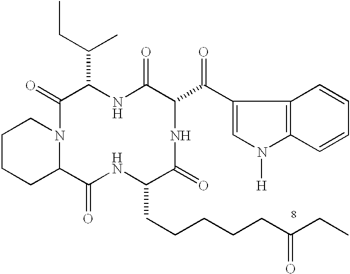

Example 102 was prepared by the following procedure. At RT, 16.1 mg DDQ was added to 20 mg N-desmethoxy apicidin in 1.1 mL 9:1 MeCN:H₂O to form a dark purple solution, which became blood-red over 30 min. The solution was aged at 0° C. for 12 h. The solution was purified without workup by RP-HPLC using 4:6 MeCN:H₂O as eluant. This yielded 15 mg of Example 102 which was characterized by ¹H NMR and MS [m/z: 608 (M⁺+1)].

### EXAMPLE 103

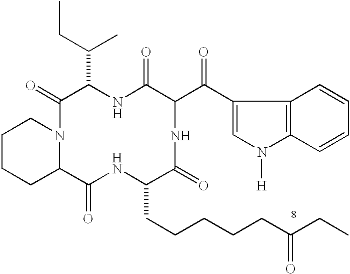

Example 103 was prepared by the following procedure. At RT, 1.5 μL Et₃N was added to 6 mg cyclo(beta-oxo-L-Trp-L-Ile-D-Pip-L-2-amino-8-oxo-decanoyl) in 0.5 mL CH₂Cl₂. After 10 min, the solution was purified without workup by RP-HPLC using 1:1 MeCN:H₂O as eluant. This yielded 3 mg pure Example 103, which was characterized by ¹H NMR and MS [m/z: 608 (M⁺+1)].

### EXAMPLES 104A AND 104B

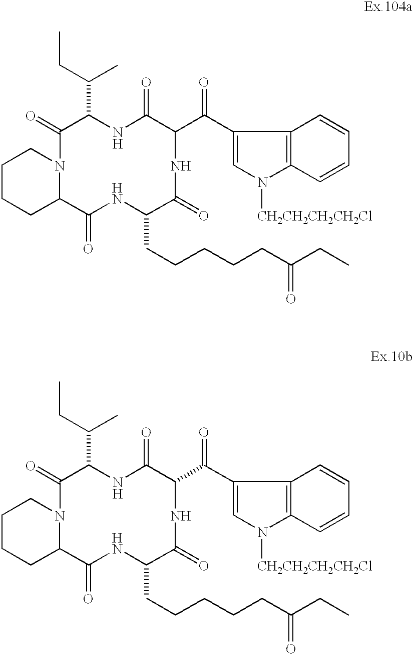

Examples 104a and 104b were prepared by the following procedure. At RT, 0.14 mL BrCH₂CH₂CH₂CH₂Cl, 0.5 g nBu₄NI and 25 mg 95% NaH were added to 300 mg beta-oxo-N-desmethoxy apicidin in 0.5 mL DMF containing 0.25 mL HMPA. The solution was degassed with bubbling N₂ for 4 min and then heated to 100° C. for 90 min. The solution was then cooled to RT, poured into saturated brine/saturated NaHCO₃, extracted with CH₂Cl₂ and dried with Na₂SO₄. Following PTLC (2×1500 μm plates) on silica gel using 1:3:96 NH₃:MeOH:CHCl₃ as eluant, a pure mixture of Example 104a and Example 104b was obtained. The pure products were characterized by ¹H NMR and MS [m/z: 698.5 (M+1) for each isomer]. The yield was 150 mg D-Trp isomer and 120 mg L-Trp isomer. TLC: R_{f}=0.42 for D-Trp isomer and 0.25 for L-Trp isomer (2:3 acetone:hexanes).

### EXAMPLES 105A AND 105B

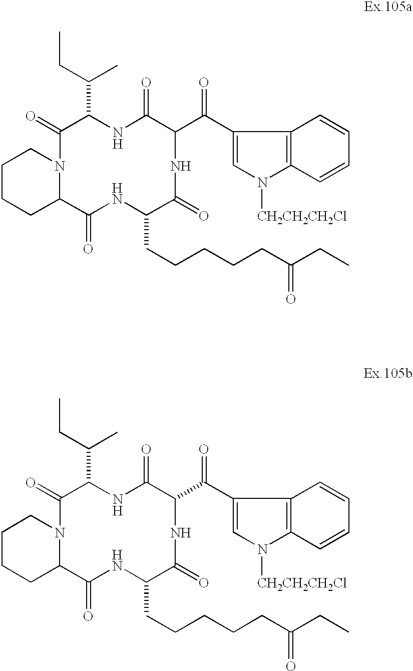

Examples 105a and 105b were prepared by adding 0.12 mL BrCH₂CH₂CH₂Cl, 0.5 g nBu₄NI and 25 mg 95% NaH at RT to 300 mg beta-oxo-N-desmethoxy apicidin in 0.5 mL DMF containing 0.25 mL HMPA. The solution was degassed with bubbling N₂ for 4 min and then heated to 100° C. for 90 min. The solution was cooled to RT, poured into 1:1 saturated brine:saturated NaHCO₃, extracted with CH₂Cl₂ and dried with Na₂SO₄. Following PTLC (2×1500 μm plates) on silica gel using 1:3:96 NH₃:MeOH:CHCl₃ as eluant, a pure mixture of Examples 105a and 105b were obtained which were characterized by ¹H NMR and MS [m/z: 684.5 (M⁺+1) for each isomer]. Yield: 120 mg D-Trp isomer and 80 mg L-Trp isomer. TLC: R_{f}=0.55 for D-Trp isomer and 0.27 for L-Trp isomer (2:3 acetone:hexanes).

### EXAMPLES 106A AND 106B

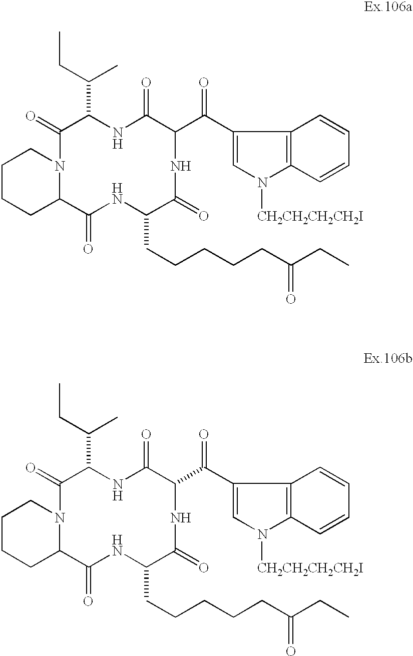

Examples 106a and 106b were prepared by adding 516 mg NaI to 120 mg cyclo(N-(4-chloro-n-butyl)-beta-oxo-D-Trp-L-Ile-D-Pip-L-2-amino-8-oxo-decanoyl) in 2.2 mL anhydrous MeCN. The resulting solution was heated to 60° C. for 12 h. The solution was cooled to RT and diluted with 1:1 brine:saturated NaHCO₃, extracted with CH₂Cl₂ and dried with Na₂SO₄. This yielded 100 mg of a mixture of Example 106a and 106b which was characterized by ¹H NMR and MS [m/z: 790.5 (M⁺+1) for each isomer] without purification. TLC: R_{f}=0.58 for D-Trp isomer and 0.41 for L-Trp isomer (1:3:96 NH₄:MeOH:CHCl₃)

### EXAMPLES 107A AND 107B

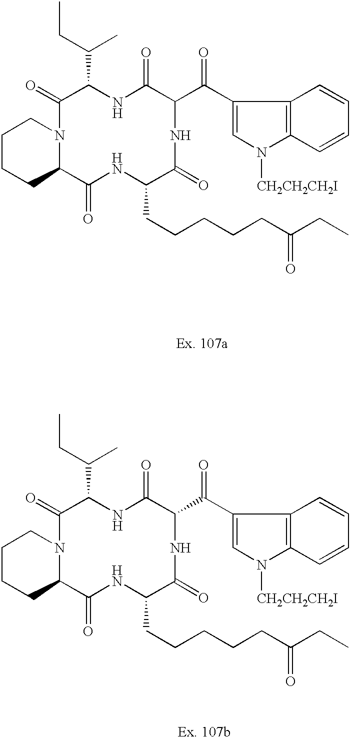

Examples 107a and 107b were prepared by adding 350 mg NaI to 80 mg cyclo(N-(4-chloro-n-propyl)-beta-oxo-D-Trp-L-Ile-D-Pip-L-2-amino-8-oxo-decanoyl) in 1.5 mL anhydrous MeCN. The resulting solution was heated to 60° C. for 12 h. The solution was cooled to RT, diluted with 1:1 brine:saturated NaHCO₃, extracted with CH₂Cl₂ and dried with Na₂SO₄. This yielded 70 mg of a mixture of Example 107a and Example 107b which were characterized by ¹H NMR and MS [m/z: 776.5 (M⁺+1) for each isomer] without purification. TLC: R_{f}=0.53 for D-Trp isomer and 0.42 for L-Trp isomer (1:3:96 NH₄OH:MeOH:CHCl₃).

### EXAMPLES 108A AND 108B

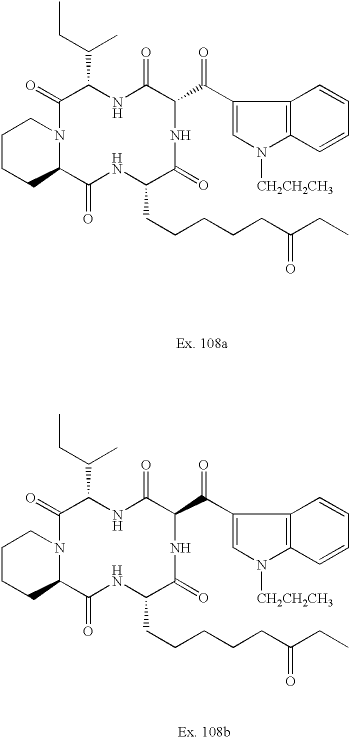

Examples 108a and 108b were prepared by adding 30 mg MgBr₂.Et₂O, and 30 μL nBu₃SnH to 40 mg of an ˜1:1 mixture of cyclo(N-(3-iodo-n-propyl)-beta-oxo-D-Trp-L-Ile-D-Pip-L-2-amino-8-oxo-decanoyl) and cyclo(N-(3-iodo-n-propyl)-beta-oxo-L-Trp-L-Ile-D-Pip-L-2-amino-8-oxo-decanoyl) in 0.5 mL CH₂Cl₂. The resulting solution was cooled to −78° C. Next, 100 μL Et3B was added followed by 500 μL oxygen gas via syringe over 2 h. The reaction was quenched by the addition of 1:1 brine:saturated NaHCO₃ at −78° C. The solution was then warmed to RT, partitioned with CH₂Cl₂ and the organic layer dried with Na₂SO₄. The solution was concentrated under reduced pressure and the residue partitioned between hexanes:MeCN (1:3). The MeCN layer was washed (3×) with hexanes and the MeCN layer concentrated under reduced pressure. Pure products were obtained following PTLC (1×1000 μm plate) on silica gel using 1:3:96 NH₄OH:MeOH:CHCl₃ as eluant. Pure products were characterized by ¹H NMR and MS [m/z: 650.6 (M⁺+1) for each isomer]. Yield: 14 mg D-Trp isomer and 14 mg L-Trp isomer. TLC: R_{f}=0.69 for D-Trp isomer and 0.51 for L-Trp isomer (1:3:96 NH₄OH:MeOH:CHCl₃).

### EXAMPLE 109

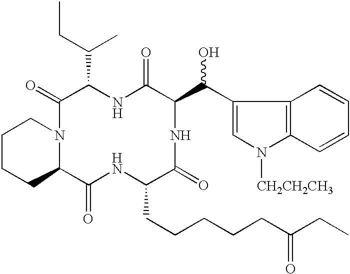

Example 109 was prepared by the following procedure. At RT, 5 mg 2,2′-azobisisobutyronitrile and 38 μL nBu₃SnH were added to a 22 mg mixture of cyclo(N-(3-iodo-n-propyl)-beta-oxo-D-(and L, ˜1:1)-Trp-L-Ile-D-Pip-L-2-amino-8-oxo-decanoyl) in 0.6 mL toluene. Nitrogen was bubbled through the solution for 5 min and it was then heated to 100° C. for 2 h. The solution was cooled to RT, concentrated under reduced pressure and the residue partitioned between hexanes:MeCN (1:3). The MeCN layer was washed (3×) with hexanes and the MeCN layer concentrated under reduced pressure. Following PTLC on silica gel using 4:6 acetone:hexanes as eluant, 10 mg pure Example 109 was obtained which was characterized by ¹H NMR and MS [m/z: 652.7 (M⁺+1)]. TLC: R_{f}=0.50 and 0.43 (mixture of beta-hydroxy isomers) (1:3:96 NH₄OH:MeOH:CHCl₃).

### EXAMPLE 110

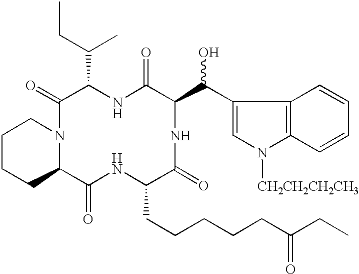

Example 110 was prepared by the following procedure. At RT, 6 mg 2,2′-azobisisobutyronitrile and 52 μL nBu₃SnH was added to 31 mg of a ˜1:1 mixture of cyclo(N-(4-iodo-n-butyl)-beta-oxo-D-Trp-L-Ile-D-Pip-L-2-amino-8-oxo-decanoyl) and cyclo(N-(4-iodo-n-butyl)-beta-oxo-L-Trp-L-Ile-D-Pip-L-2-amino-8-oxo-decanoyl) in 0.8 mL toluene. Nitrogen was bubbled through the solution for 5 min and it was then heated to 100° C. for 2 h. The solution was cooled to RT, concentrated under reduced pressure and the residue partitioned between hexanes:MeCN (1:3). The MeCN layer was washed (3×) with hexanes and the MeCN layer concentrated under reduced pressure. Example 110 was characterized by ¹H NMR without purification. TLC: R_{f}=0.66 (1:3:96 NH₄OH:MeOH:CHCl₃).

### EXAMPLES 111A AND 111B

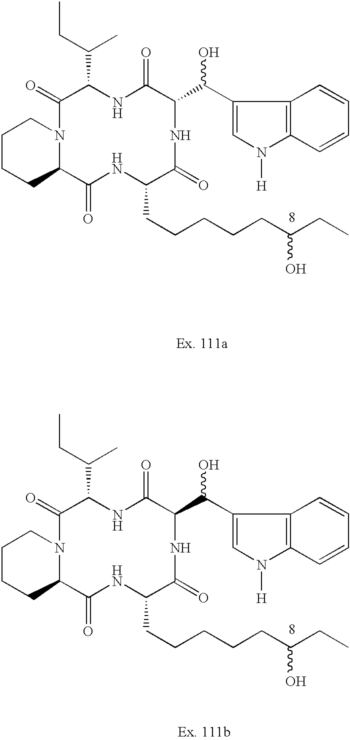

Examples 111a and 111b were prepared by adding 1 mg NaBH₄ to 3 mg beta-oxo-N-desmethoxy apicidin in 0.25 mL EtOH at 0° C. After 2.5 h at RT and 10 h at 0° C., the solution was poured into saturated NH₄Cl, extracted with 3:1 EtOAc:iPrOH and dried with Na₂SO₄. Following RP-HPLC using gradient elution (2:3 to 1:1 MeCN:H₂O), a mixture of pure Example 111a and 111b were obtained which was characterized by ¹H NMR and MS [m/z: 594 (M⁺−H₂O) for both isomers]. TLC: R_{f}=0.50 for D-Trp isomer and 0.28 for L-Trp isomer (1:9:90 NH₄OH:MeOH:CHCl₃). HPLC: t_{R}=3.9 min for D-Trp isomer and 3.5 min for L-Trp isomer (1:1 MeCH:H₂O, 1.5 mL/min, Zorbax™ RX-C8).

### EXAMPLES 112A AND 112B

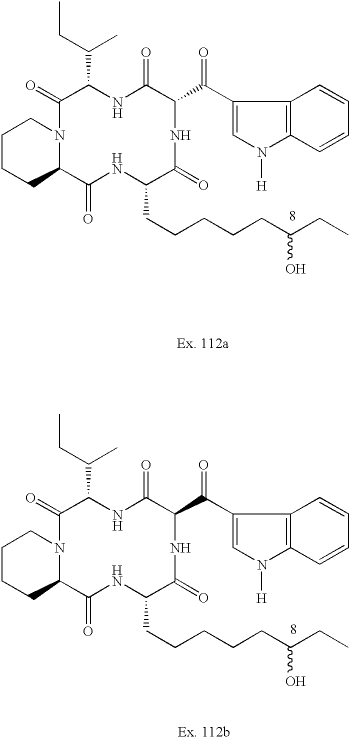

Examples 112a and 112b were prepared by adding 5.8 mg CeCl₃-6H₂O to 10 mg cyclo(beta-oxo-L-Trp-L-Ile-D-Pip-L-2-amino-8-oxo-decanoyl) at RT in 0.2 mL MeOH. After 5 min, the solution was cooled to 0° C. and 0.6 mg NaBH₄ was added. The solution was poured into saturated NH₄Cl, extracted with EtOAc and dried with Na₂SO₄. Following RP-HPLC using 1:1 MeCN:H₂O as eluant, a pure mixture of 0.7 mg Example 112a and 1.3 mg Example 112b was obtained which was characterized by ¹H NMR and MS [m/z: 609 (M⁺+1) for each isomer].

### EXAMPLES 113A AND 113B

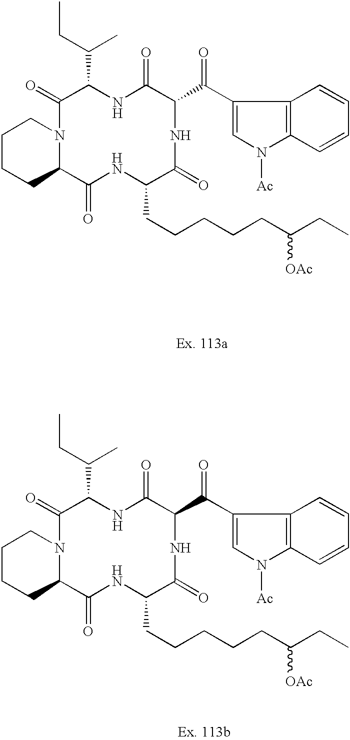

Examples 113a and 113b were prepared by adding 14 mg DMAP and 0.533 mL Ac₂O to 700 mg cyclo(beta-oxo-L-Trp-L-Ile-D-Pip-L-2-amino-8-hydroxy-decanoyl) in 115 mL dichloroethane at RT. After 8 h, the mixture was poured into saturated NH₄Cl, extracted with CH₂Cl₂ and dried with Na₂SO₄. Following preparative chromatotron (4 μm plate) on silica gel using 2:8 to 4:6 acetone:hexanes gradient elution as eluant, 8 mg of a mixture of Examples 113a and 113b was obtained. Pure epimeric products were characterized by ¹H NMR and MS. D-Trp isomer: yield: 140 mg; TLC: R_{f}=0.71 (1:1 acetone:hexanes); MS [m/z: 694.4 (M⁺+1)]. L-Trp isomer: yield: 110 mg; TLC: R_{f}=0.57 (1:1 acetone:hexanes); MS [m/z: 694.5 (M⁺+1)].

### EXAMPLE 114

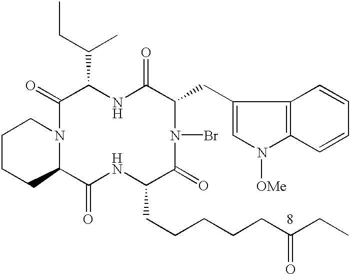

Example 114 was prepared by the following procedure. At RT, 28.5 mg N-bromosuccinamide and 1.2 mg benzoyl peroxide was added to 100 mg apicidin in 5.3 mL CCl₄. Nitrogen then was bubbled through the solution for 5 min. The solution was refluxed for 15 min and then cooled to RT. Following PTLC on silica gel (3×1000 μm plates) using 1:3:96 NH₄OH:MeOH:CHCl₃ (one development) followed by 4:6 acetone:hexanes (two developments) as eluant, 62 mg pure Example 114 was obtained which was characterized by ¹H NMR and MS [m/z: 704 (M⁺+1)]. RP-HPLC: t_{R}=5.02 min (apicidin: t_{R}=4.82 min), 6:4 MeCN:H₂O, 1.5 mL/min.

### EXAMPLES 115A AND 115B

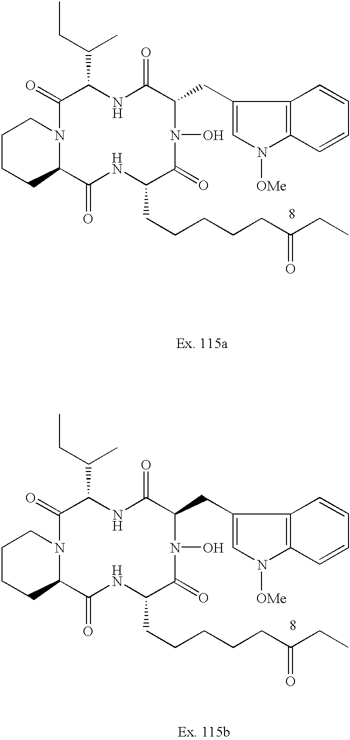

Example 115a (mobile product A) and Example 115b (polar product B) were prepared by the following methods E and F.

Method E

At 0° C., 10 mg Example 114 was added to 4 mg AgBF₄ in 250 μL 3:1 DMSO:CH₂Cl₂. After aging for 10 min (at this point, TLC showed the disappearance of the starting bromide), 10 μL Et₃N was added and the solution aged for an additional hour. The reaction was quenched by the addition of water. The mixture was then extracted with CH₂Cl₂ and dried with Na₂SO₄. Following PTLC on silica gel (1×250 μm plate) using 1:1 acetone:hexanes as eluant, a pure mixture of Examples 115a and 115b was obtained which were characterized by ¹H NMR and MS [m/z: 640 (M⁺+1) for both isomers]. TLC: R_{f}=0.48 Example 115a (mobile product A) and 0.41 Example 115b (polar product B), 1:1 acetone:hexanes.

Method F

At RT, 12 mg NaHCO₃ was added to 43 mg apicidin in CH₂Cl₂, followed by 18 mg 85% MCPBA. The resulting solution was vigorously stirred for 12 h. The solution was then poured into saturated NaHCO₃(aq), extracted with CH₂Cl₂ and dried with Na₂SO₄. Following PTLC on silica gel (1×250 μm plate) using 1:1 acetone:hexanes, pure Example 115a was obtained which was identical in all respects to Example 115a, mobile product A, from Method E above.

### EXAMPLE 116

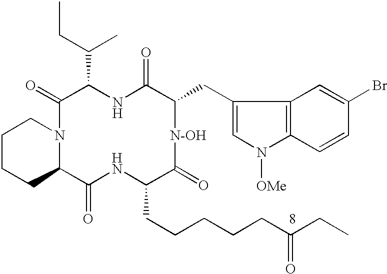

Example 116 was prepared by following the general procedure of Example 115a, method E. Starting with 10 mg of Example 114, 4 mg of Example 116 was prepared which was characterized by ¹H NMR and MS [m/z: 718.6 (M⁺+1)].

### EXAMPLE 117

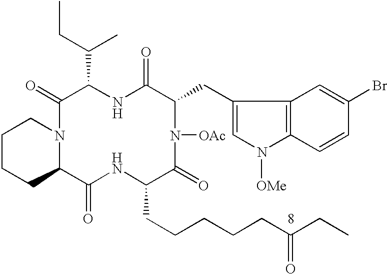

Example 117 was prepared by adding 4 μL Ac₂O to 5.4 mg of Example 115b at RT in 375 μL ClCH₂CH₂Cl, followed by the addition of 0.3 mg DMAP. After 1.5 h, the volatiles were removed under a stream of nitrogen. Following PTLC on silica gel (1×250 μm plate) using 1:1 acetone:hexanes as eluant, 6 mg pure Example 117 which was characterized by ¹H NMR and MS [m/z: 762 (M⁺+1)].

### EXAMPLE 118

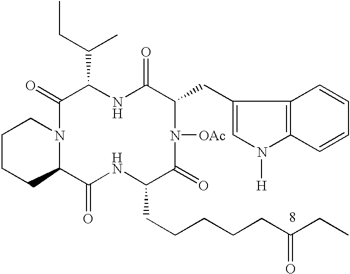

Example 118 was prepared by the following procedure. To 5 mg of Example 117 at RT in 1 mL, CH₂Cl₂ was added 1 mg Pd(OH)₂ and a hydrogen atmosphere was established (balloon pressure). After aging for 2 h, the solution was filtered and concentrated under reduced pressure. Following flash chromatography on silica gel using 1:9:90 NH₄:MeOH:CHCl₃, 3.5 mg pure Example 118 was obtained which was characterized by ¹H NMR and MS [m/z: 652 (M⁺+1)].

### EXAMPLE 119

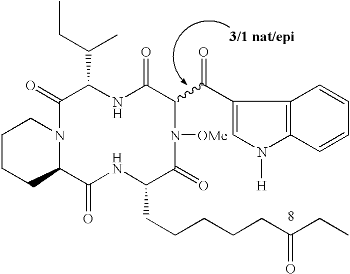

Example 119 was prepared by the following procedure. To 25 mg beta-oxo-N-desmethoxy-apicidin at RT in 1.5 mL MeOH was added 15 μL pyridine followed by the addition of 126 mg Pb(OAc)₄. After aging for 48 h, the solution was cooled to 0° C. and saturated Na₂S₂O₃(aq) was added. The solution was poured into saturated NH₄Cl(aq):brine (1:1), extracted with iPrOH:CHCl₃ (3:7) and dried with Na₂SO₄. The solution was filtered and concentrated under reduced pressure. Following flash chromatography on silica gel using 1:9:90 NH₄:MeOH:CHCl₃, 36 mg pure Example 119 was obtained which was characterized by ¹H NMR and MS [m/z: 638 (M⁺+1)].

### EXAMPLE 120

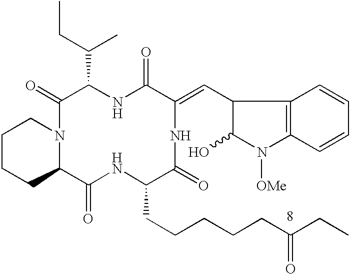

Example 120 was prepared by the following procedure. To 81 mg of Example 114 in 6 mL THF:H₂O at RT was added 141 mg basic Al₂O₃ and 191 mg Ag₂CO₃. The solution was warmed to 50° C. for 5 h, then cooled to RT. The mixture was partitioned between water and CH₂Cl₂, the layers separated, the organic layer dried with Na₂SO₄ and then filtered through Celite. Following PTLC (1×500 μm plate) using 1:3:96 NH₄OH:MeOH:CHCl₃ as eluant, pure Example 120 was obtained which was characterized by ¹H NMR and MS [m/z: 640.5 (M⁺+1)].

### EXAMPLE 121

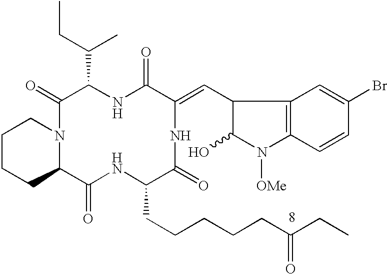

Example 121 was prepared by following the general procedure of Example 120, and utilizing the dibromide Example 126 as the starting material. The product thus obtained was characterized by ¹H NMR and MS [m/z: 720 (M⁺+1)].

### EXAMPLE 122

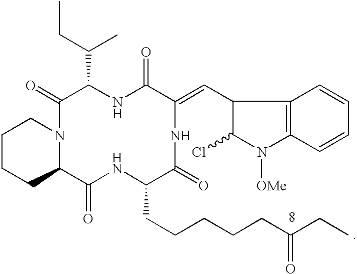

Example 122 was prepared by the following procedure. To 200 μL CH₂Cl₂ at −78° C. was added 6 μL oxallyl chloride (2M solution in CH₂Cl₂) followed by the addition of 2 μL DMSO. After 5 min, 3.3 mg of Example 120 (as a solution in 50 μL CH₂Cl₂) was added to the DMSO/oxallyl chloride solution. After aging for 15 min, 14 μL Et₃N was added and the solution as warmed to 0° C. The reaction was then quenched by the addition of water, extracted with CH₂Cl₂ and dried with Na₂SO₄. Following PTLC on silica gel (1×500 μm plate) using 1:3:96 NH₄OH:MeOH:CHCl₃ as eluant, pure Example 122 was obtained which was characterized by ¹H NMR and MS [m/z: 658 (M⁺+1)].

### EXAMPLE 123

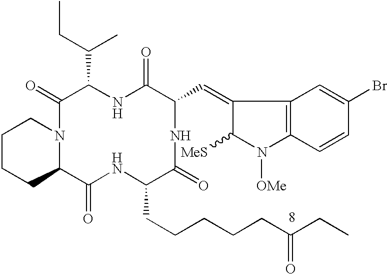

Example 123 was prepared by mixing 28 mg of Example 126 in 1.5 mL DMF at RT with 13 mg NaSMe. The mixture was then warmed to 50° C. After 1 h, the solution was poured into water, extracted with CH₂Cl₂ and dried with Na₂SO₄. Following PTLC purification on silica gel (1×25 μm plate) using 4:6 acetone:hexanes as eluant (two developments). Pure Example 123 was obtained which was characterized by ¹H NMR and MS [m/z: 748 (M⁺+1)].

### EXAMPLE 124

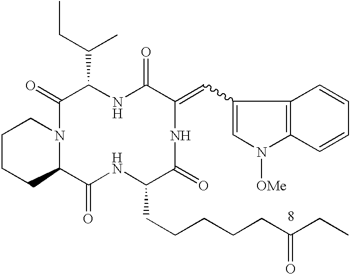

Example 124 was prepared by adding 5.4 mg KSAc to 11 mg of Example 114 in 260 μL DMF at 0° C. After aging the solution for 48 h, it was warmed to RT and aged an additional 20 h. The solution was poured into water, extracted with CH₂Cl₂ and dried with Na₂SO₄. Following PTLC on silica gel using 4:6 acetone:hexanes as eluant, the product thus obtained was characterized by ¹H NMR and MS [m/z: 622 (M⁺+1)].

### EXAMPLE 125

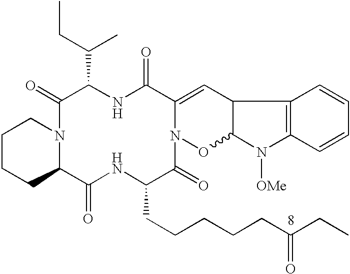

Example 125 was prepared by adding 5 mg DDQ to 5 mg of Example 115a (mobile product A) at RT in 200 μL THF. The resulting solution was warmed to 65° C. After aging for 20 h, an additional 5 mg DDQ was added. After an additional 6 h, the volatiles were removed at ambient temperature under reduced pressure. Methylene chloride was added, the solution was filtered and the filtrate was loaded onto a preparative TLC plate (1×250 μm plate, silica gel). Following PTLC purification using 4:6 acetone:hexanes as eluant, the pure Example 125 thus obtained was characterized by ¹H NMR and MS [m/z: 608.6 (M⁺+1)].

### EXAMPLE 126

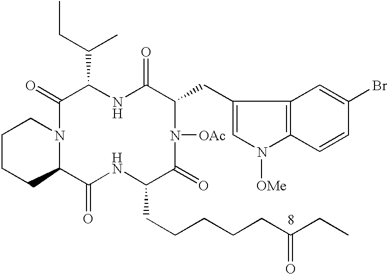

Example 126 was prepared by the following procedure. At RT, 86 mg N-bromosuccinamide was added to 100 mg apicidin in 5.3 mL CCl₄, followed by the addition of 1.2 mg benzoyl peroxide. The resulting solution was purged with vigorous nitrogen bubbling for 5 min. The solution was heated to reflux for 45 min and then cooled to RT. The volatiles were removed under reduced pressure and pure Example 126 was obtained following PTLC purification on silica gel (1500 μm plate) using 1:3:96 NH₄OH:MeOH:CHCl₃ as eluant. The dibromide Example 126 product thus obtained was characterized by ¹H NMR and MS [m/z: 780 (M⁺+1)]. TLC: R_{f}=0.49 (1:3:96 NH₄OH:MeOH:CHCl₃). HPLC: t_{R}=10.02 min, mL/min, 6:4 MeCN:H₂O, Zorbax™ RX-8).

### EXAMPLES 127A AND 127B

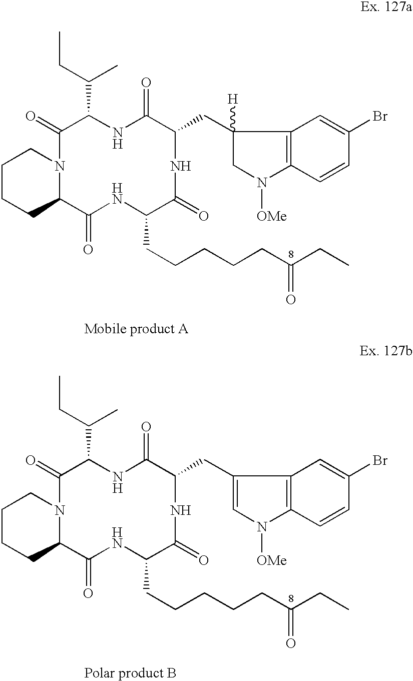

Examples 127a and 127b were prepared by adding 0.32 mL DMF, and 0.32 mL 1:1 saturated NaHCO₃:H₂O to 10 mg of Example 126, followed by the addition of 4.5 mg Na₂S₂O₄. The milky white solution thus formed was aged at RT for 24 h. Then, 2 mL Acetonitrile was added, and the solids were removed by filtration. This yielded 1 mg pure Example 127a (mobile product A) and 4 mg pure Example 127b (polar product B) following RP-HPLC using 1:1 MeCN:H₂O as eluant. Both products were characterized by ¹H NMR and MS.

Example 127a, mobile product A: MS: [m/z: 704 (M⁺+1)]; TLC: R_{f}=0.75 (1:1 acetone:hexanes); HPLC: t_{R}=8 min, 2 mL/min, 1:1 MeCN:H₂O, Zorbax™ RX-8).

Example 127b, polar product B: MS: [m/z: 702 (M⁺+1)]; TLC: R_{f}=0.60 (1:1 acetone:hexanes); HPLC: t_{R}=7 min, 2 mL/min, 1:1 MeCN:H₂O, Zorbax™ RX-8).

### EXAMPLE 128

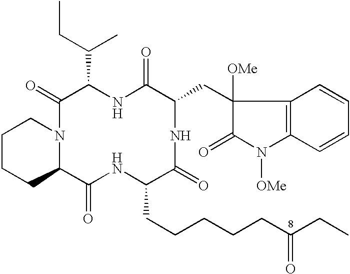

Example 128 was prepared by the following procedure. At 0° C., 6 mg N-bromosuccinamide was added to 13 mg apicidin in 1 mL CH₂Cl₂ and 0.5 mL MeOH. After 4 min, 1 mL saturated Na₂SO₃(aq) was added, followed by 1 mL brine. The solution was extracted with EtOAc and dried with Na₂SO₄. Partially purified product was obtained following PTLC on silica gel (1×1500 μm plate) using 1:2 acetone:hexanes as eluant. Pure Example 128 was subsequently obtained following flash chromatography on silica gel using 1:2 acetone:hexanes as eluant. The Example 128 thus obtained was characterized by ¹H NMR and MS [m/z: 670.4 (M⁺+1)].

### EXAMPLES 129A AND 129B

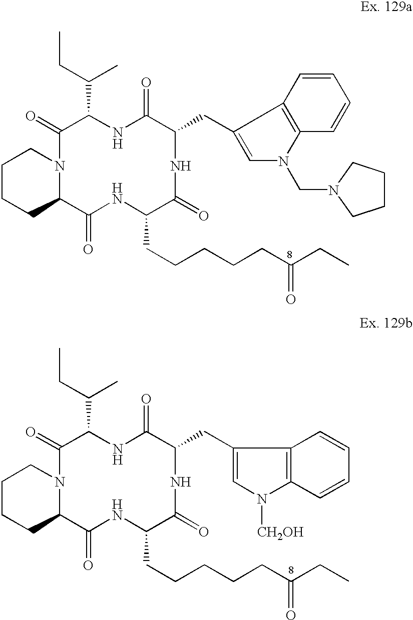

Examples 129a and 129b were prepared by the following procedure. To 10 mg N-desmethoxy apicidin at RT in 0.1 mL DMF was added 3 μL 37% formaldehyde(aq) and 3 μL pyrrolidine. After 48 h, the reaction was quenched with saturated NaHCO₃, extracted with EtOAc and dried with Na₂SO₄. Pure 2 mg of the pyrrolidino Example 129a (R_{f}=0.2) and 2 mg of the hydroxymethyl Example 129b (R_{f}=0.1) was obtained following PTLC (1:3:96 NH₄OH:MeOH:CHCl₃, R_{f}=0.2) as eluant. The pure products were characterized by ¹H NMR and MS [m/z: 624 (M⁺+1) for the pyrrolidino Example 129a and 677 (M+1) for the hydroxymethyl Example 129b].

### EXAMPLE 130

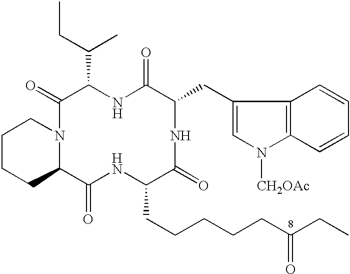

Example 130 was prepared by the following procedure. To 5 mg N-desmethoxy-N-hydroxymethyl apicidin in 0.16 mL pyridine at RT was added 0.63 mL acetyl chloride and one crystal of DMAP. After 12 h, the reaction was quenched with saturated NH₄Cl, extracted with EtOAc and dried with Na₂SO₄. Following RP-HPLC using a linear gradient of 4:6 to 1:0 MeCN:H₂O as eluant, pure Example 130 was obtained which was characterized by ¹H NMR and MS [m/z: 666 (M⁺+1)].

### EXAMPLE 131

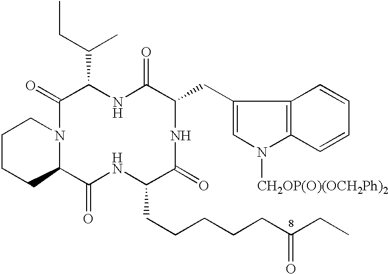

Example 131 was prepared by the following procedure. To 9 mg N-desmethoxy-N-hydroxymethyl apicidin in CH₂Cl₂ at 0° C. was added 13 μL EtN(iPr)₂ followed by the addition of 43 μL (PhCH₂O)₂P(O)Cl. After 30 min at 0° C., 0.4 mg DMAP was added and the solution was aged for 1.5 h at 0° C., followed by 2.5 h at RT. The reaction was quenched by the addition of water, extracted with CH₂Cl₂ and dried with Na₂SO₄. Following preparative RP-HPLC using a linear gradient of 4:6 [symbol] 1:0 MeCN:H₂O as eluant, 0.3 mg pure Example 131 was obtained which was characterized by ¹H NMR and MS [m/z: 884 (M+1)].

### EXAMPLES 132A AND 132B

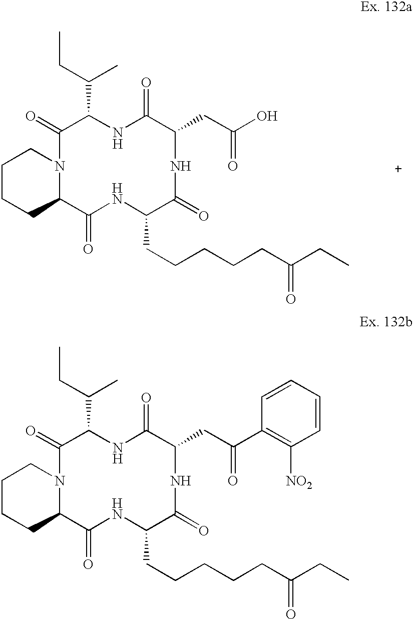

Examples 132a and 132b were prepared by the following methods G, H and I.

Method G

To 100 mg apicidin in 4 mL MeCN and 3 mL CH₂Cl₂ at RT was added 800 mg NaIO₄ in 10 mL water, followed by the addition of 10 mg RuCl₃. The solution was then aged overnight. The solution was poured into brine, acidified with glacial acetic acid and filtered to remove particulates. The solids were rinsed with CH₂Cl₂ and the solution was extracted with CH₂Cl₂. The combined organic layers were dried with Na₂SO₄, filtered, and concentrated under reduced pressure. Pure 52 mg carboxylic acid Example 132a was obtained following preparative RP-HPLC using gradient elution (1:4 to 1:1 MeCN:H₂O, 50 min linear ramp). Example 132a obtained was characterized by ¹H NMR and MS [m/z: 523.2 (M⁺+1)]. Also obtained from this reaction was the nitrophenylketone apicidin analog, Example 132b, which was characterized by ¹H NMR and MS [m/z: 628.2 (M⁺+1)].

Method H

To a solution containing 0.3 mg RuCl₃.xH₂O and 50 mg N-desmethoxy apicidin in 2 mL 1:1 MeCN:CCl₄ was added 324 mg NaIO₄ (as a solution in 1 mL H₂O). After 45 h, the resulting green solution was partitioned between 1:1 brine:saturated NH₄Cl and 3:7 iPrOH:CHCl₃. The organic layer then was dried with Na₂SO₄. The solution was concentrated under reduced pressure to yield 60 mg crude product.

Method I

To 9 mg cyclo(L-Asp-L-Ile-D-Pip-L-2-amino-8-oxo-decanoyl), methyl ester in 1 mL 3:1:1 THF:MeOH:H₂O at 0° C. was added 50 μL 1M LiOH. After 1 h at 0° C., followed by 2 days at RT, the solution was filtered through a reversed-phase plug (0.5 g C-18) with MeOH as eluant, concentrated under reduced pressure, and purified without workup by RP-HPLC using gradient elution (10 min ramp from 5:95 MeCN:H₂O to 25:75 MeCN:H₂O, then 60 min ramp to 100% MeCN).

### EXAMPLE 133

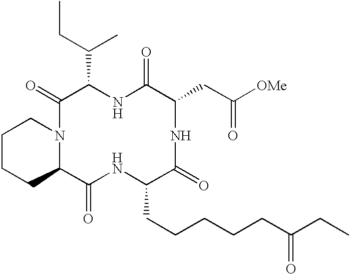

Example 133 was prepared by adding 1 mL Me₃SiCH═N₂ (0.5M solution in hexanes) to 12 mg of the carboxylic acid product of Example 132a in 4 mL 2:1 MeOH:Et₂O at RT. After 20 min, the solution became homogenous and 0.25 mL glacial acetic acid was added. The solution was poured into brine, extracted with CH₂Cl₂ and dried with Na₂SO₄. The solution was filtered and concentrated under reduced pressure. Pure Example 133 was obtained following PTLC on silica gel (1×1000 μm plate) using 1:1 acetone: hexanes as eluant. The methyl ester Example 133 thus obtained was characterized by ¹H NMR and MS [m/z: 537.5 (M⁺+1)].

### EXAMPLE 134

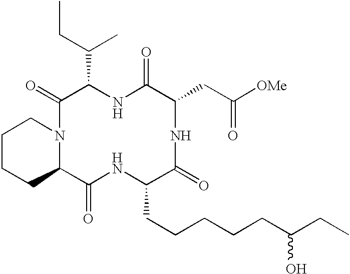

Example 134 was prepared by adding 9.6 mg NaBH₄ to 120 mg of Example 133 in 7 mL THF at 0° C. After aging for 3 h, the reaction was quenched by the addition of saturated NH₄Cl(aq), extracted with CH₂Cl₂ and dried with Na₂SO₄. Following PTLC using 4:6 acetone:hexanes (R_{f}=0.53) as eluant, 117 mg of pure Example 134 was obtained which was characterized by ¹H NMR.

### EXAMPLE 135

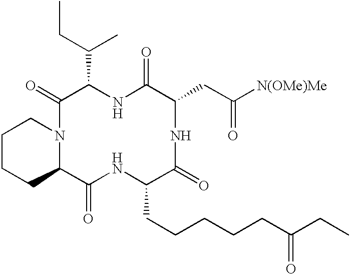

Example 135 was prepared by the following methods J and K.

Method J

To 50 mg of Example 132a in 2 mL CH₂Cl₂ at RT was added sequentially 14 μL Et₃N followed by 8 μL MeSO₂Cl. After aging for 2 h, 18 mg solid HCl.HN(OMe)Me was added. After an additional hour, the volatiles were removed under reduced pressure. Following flash chromatography on silica gel using 1:2:97 NH₄OH:MeOH:CHCl₃ as eluant, 1.9 mg pure Example 135 was obtained which was characterized by ¹H NMR and MS.

Method K

To 20 mg of Example 132a in 1 mL THF at −78° C. was added 10.6 mg HCl.HN(OMe)Me followed by the dropwise addition of 112 μL iPrMgBr (2M solution in THF). The resulting solution was slowly allowed to warm to 4° C. and was aged for 12 h. The reaction was quenched by addition of 1 mL saturated NH₄Cl(aq), extracted with CH₂Cl₂ and dried with Na₂SO₄. Following flash chromatography on silica gel using 1:2:97 NH₄OH:MeOH:CHCl₃ as eluant, 11 mg pure Example 135 was obtained which was characterized by ¹H NMR and MS.

### EXAMPLE 136

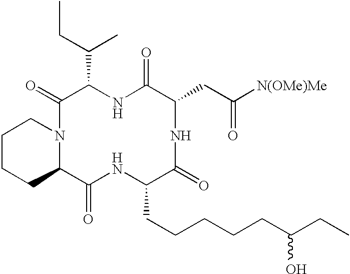

Example 136 was prepared from 117 mg of Example 134 using Method K as described in Example 135. This resulted in 76 mg of Example 136 (R_{f}=0.46, 1:9:90 NH₄OH:MeOH:CHCl₃) which was characterized by ¹H NMR and MS [m/z: 568 (M+1)].

### EXAMPLE 137

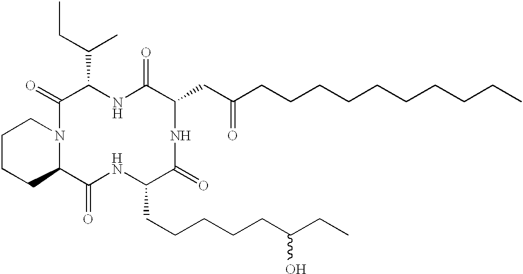

Example 137 was prepared by the following procedure. To 11 mg cyclo(N-O-methyl-N-meth yl-L-Asp-L-Ile-D-Pip-L-2-amino-8-hydroxy-decanoyl) in 0.39 mL THF and 80L HMPA at 0° C. was added 388 μL n-C₁₀H₂₁MgBr (1M in Et₂O). The solution was warmed immediately to RT and aged for 12 h. The solution was poured into saturated NH₄Cl(aq), partitioned with THF and dried with Na₂SO₄. Following PTLC (1×500 μm plate) on silica gel using 1:9:90 NH₄OH:MeOH:CHCl₃ as eluant, 2.5 mg pure Example 137 was obtained which was characterized by ¹H NMR and MS [m/z: 649 (M⁺+1)].

### EXAMPLE 138

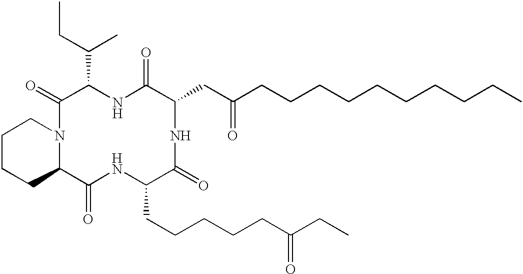

Example 138 was prepared by adding 5 μL pyridine to 2 mg of Example 137 in 0.35 μL CH₂Cl₂ at 23° C., followed by the addition of 7 mg Dess-Martin periodinane. After 1.5 h, the solution was poured into 1:1 saturated NaHCO₃: 10% NaHSO₃, aged for 10 min, then extracted with CH₂Cl₂, and dried with Na₂SO₄. Following PTLC (1×250 μm plate) on silica gel using 1:3:96 NH₄OH:MeOH:CHCl₃ as eluant, 1.5 mg pure Example 138 was obtained which was characterized by 1H NMR and MS [m/z: 647 (M⁺+1)].

### EXAMPLES 139A-139J

Following the general procedures described in Examples 137 and 138, and utilizing the appropriate starting compounds and reactants—particularly the appropriate nucleophile for the R₁ group—which would be clear to one in the art, the following compounds were prepared:

TABLE 7

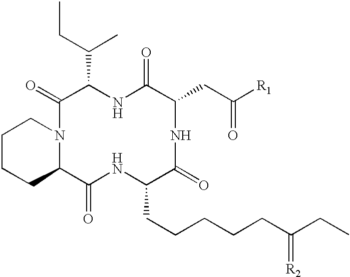

| Example | R₁ Group | R₂ Group | Mass Spec |
|---|---|---|---|
| 139a | CH₂Ph | H, OH | 599 (M⁺ + 1) |
| 139b | CH₂Ph | ═O | 597 (M⁺ + 1) |
| 139c | 1-napthyl | H, OH | 635 (M⁺ + 1) |
| 139d | 1-napthyl | ═O | 633 (M⁺ + 1) |
| 139g | 5-(N-methyl-indolyl) | H, OH | 638 (M⁺ + 1) |
| 139h | 5-(N-methyl-indolyl) | ═O | 636 (M⁺ + 1) |
| 139i | tBu | H, OH | 565 (M⁺ + 1) |
| 139j | tBu | ═O | 563 (M⁺ + 1) |

### EXAMPLE 140

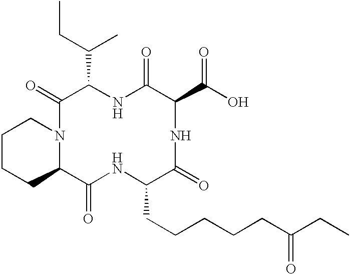

Example 140 was prepared by the following procedure. To 100 mg cyclo(beta-oxo-L-Trp-L-Ile-D-Pip-L-2-amino-8-oxo-decanoyl) at RT in 6 mL 1:1:1 MeCN:CCl₄:H₂O was added 0.7 mg RuCl₃.2H2O followed by the addition of 634 mg NaIO₄ (as a sonicated solution in 2 mL H₂O). After 30 h, the resulting tan-white heterogeneous solution was partitioned between 1:1 brine:saturated NH₄Cl and 3:7 iPrOH:CHCl₃. The organic layer was dried with Na₂SO₄ and concentrated under reduced pressure to yield 100 mg of Example 140. The crude product was characterized by ¹H NMR and MS [m/z: 526 (M⁺+NH₄)] with no additional purification.

### EXAMPLE 141

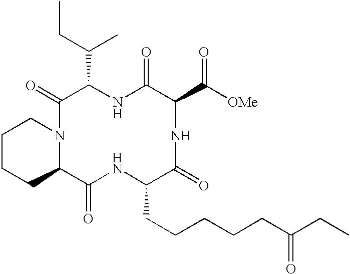

Example 141 was prepared by the following procedure. To 80 mg cyclo(D-2-amino-2-carboxy-ethanoyl-L-Ile-D-Pip-L-2-amino-8-oxo-decanoyl) in 3.3 mL 2:1 MeOH:Et₂O at RT was added 1 mL TMSCHN₂ (2M in hexanes). After 1.5 h, glacial HOAc was added dropwise until foaming ceased and the solution was partitioned between 1:1 brine:saturated NH₄Cl and CH₂Cl₂. The organic layer was dried with Na₂SO₄. Following PTLC (1×1500 μm plate) on silica gel using 3:97 HOAc:EtOAc as eluant, 28 mg pure Example 141 was obtained which was characterized by ¹H NMR and MS [m/z: 540 (M⁺+NH₄)].

### EXAMPLE 142

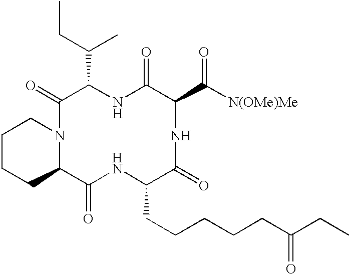

Example 142 was prepared by the following procedure. To 26.5 mg of Example 140 in 2 mL CH₂Cl₂ at RT was added 51 mg HCl.HN(OMe)Me and 13 mg DMAP, followed by the addition of 46 mg BOP. After aging for 8 h at RT, the solution was warmed to 40° C. for 12 h. Following removal of volatiles, 2 mg pure Example 142 was obtained following PTLC on silica gel using 1:1 acetone:hexanes as eluant. The product was characterized by ¹H NMR and MS [m/z: 552 (M⁺+1)].

### EXAMPLE 143

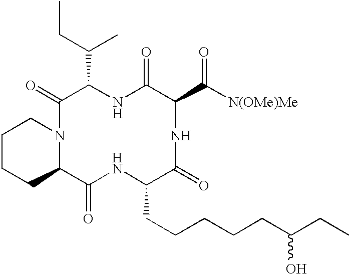

Example 143 was prepared by starting with Example 141. First, the side chain carbonyl of Example 141 was reduced as described in Example 134. The resulting intermediate compound was then treated by the procedure described in Example 135. The pure Example 143 thus obtained was characterized by ¹H NMR.

### EXAMPLES 144A-144G

Following the general procedures described above in Examples 142 and 143 (procedure of Example 142 was utilized for Example 144g, while the procedure of Example 143 was utilized for the other Examples 144a-144f), and using the appropriate materials which would be clear to one in the art, the following compounds were prepared:

TABLE 8

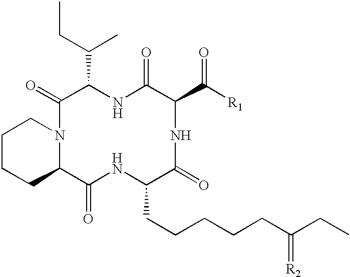

| Example | R₁ Group | R₂ Group | Mass Spec |
|---|---|---|---|
| 144a | CH₂Ph | H, OH | 602 (M⁺ + NH₄) |
| 144b | CH₂Ph | ═O | 600 (M⁺ + NH₄) |
| 144c | iPr | H, OH | 537 (M⁺ + 1) |
| 144d | iPr | ═O | 552 (M⁺ + NH₄) |
| 144e | 5-(N-methylindolyl) | H, OH | 624 (M⁺ + 1) |
| 144f | 5-(N-methylindolyl) | ═O | 622 (M⁺ + 1) |
| 144g | CH₂Ph | PhCH₂—, OH | 689 (M⁺ + 1) |

### EXAMPLE 145

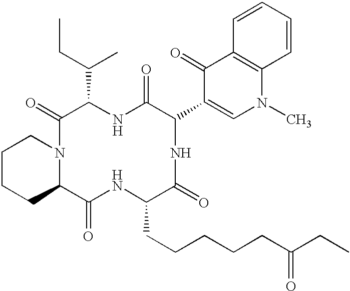

Example 145 was prepared by the following procedure. To 10 mg N-desmethoxy-N-methyl apicidin in 2.5 mL CH₂Cl₂ at −78° C. was bubbled O₃ until the solution turned light blue. The resulting solution was stirred for 10 min and then N₂ was bubbled through the solution for 5 min. Next, 250 μL Dimethylsulfide was added, the solution then warmed slowly to RT and concentrated under reduced pressure. The resulting residue was dissolved in 1:1 THF:tBuOH at 0° C., followed by the addition of 3.7 mg tBuOK. After 2 h at 0° C., the solution was poured into 1:1 water:saturated NaHCO₃, extracted with CH₂Cl₂ and dried with Na₂SO₄. Following preparative TLC on silica gel (1×500 μm plate) using 1:3:96 NH₄OH:MeOH:CHCl₃ as eluant, pure Example 145 was obtained which was characterized by ¹H NMR and MS [m/z: 622.7 (M⁺+1)]. TLC: R_{f}=0.15 (1:3:96 NH₄OH:MeOH:CHCl₃).

### EXAMPLES 146A-146F

Following the general ozonalysis procedure described for Example 145, the following compounds were prepared:

TABLE 9

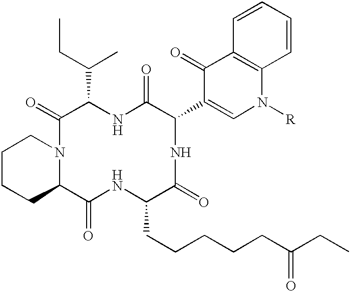

| Example | R Group | Starting Compound | Mass Spec |
|---|---|---|---|
| 145 | Me | Ex. 69 | 622.7 (M⁺ + 1) |
| 146a | H | Apicidin | 608.3 (M⁺ + 1) |
| 146b | OMe | Apicidin | 638.3 (M⁺ + 1) |
| 146c | Et | Ex. 74a | 636.8 (M⁺ + 1) |
| 146d | nPr | Ex. 74b | 650.3 (M⁺ + 1) |
| 146e | CH₂CO₂Me | Ex. 70 | 680.7 (M⁺ + 1) |
| 146f | CH₂CO₂H | Ex. 79 | 666.6 (M⁺ + 1) |

### EXAMPLE 147

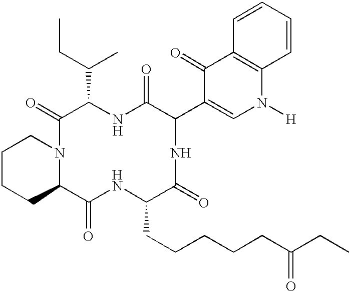

Example 147 was prepared by the following procedure. To 10 mg cyclo(L-2-amino-2-(3′-(quinol-4′-onyl))-ethanoyl-L-Ile-D-Pip-L-2-amino-8-oxo-decanoyl) in 1 mL ClCH₂CH₂Cl at RT was added 4 mg DMAP and 19 μL TEA, followed by the addition of 5 μL MeSO₂Cl. After 15 min at RT, the solution was poured into saturated NaHCO₃, extracted with CH₂Cl₂ and dried with Na₂SO₄. Following preparative TLC on silica gel (1×500 μm plate) using 1:1 acetone:hexanes as eluant, pure Example 147 was obtained which was characterized by ¹H NMR and MS [m/z: 608.5 (M⁺+1)]. TLC: R_{f}=0.43 (1:1 acetone:hexanes).

### EXAMPLES 148A AND 148B

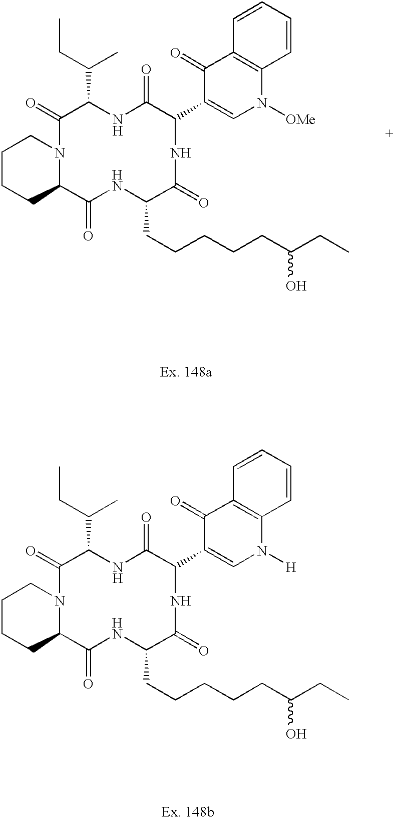

Examples 148a and 148b were prepared by adding 3 mg NaBH₄ to 20 mg cyclo(L-2-amino-2-(3′-(N-O-methyl-quinol-4′-onyl))-ethanoyl-L-Ile-D-Pip-L-2-amino-8-oxo-decanoyl) in 5 mL MeOH at 0° C. Then the cooling bath was removed promptly. After 20 min, acetone was added to quench the reaction and the solution was poured into saturated NaHCO₃, extracted with CH₂Cl₂ and dried with Na₂SO₄. Initial purification was accomplished following flash chromatography on silica gel using 1:1 acetone:hexanes as eluant. At this juncture, it was noted that the resulting product was approximately 1:1 mixture of two compounds with similar TLC R_{f} values (product A: 0.39 and Product B: 0.28 in 1:1 acetone:hexanes). Repurification by preparative TLC on silica gel (1×500 μm plate) yielded two pure products which were characterized by ¹H NMR and MS [m/z: 640.6 (M⁺+1) for Example 148a and 610.5 (M⁺+1) for Example 148b].

Example 148a: cyclo(L-2-amino-2-(3 ′-(N-O-methyl-quinol-4′-onyl))-ethanoyl-L-Ile-D-Pip-L-2-amino-8-hydroxy-decanoyl); TLC: R_{f}=0.55 (1:3:96 NH₄OH:MeOH:CHCl₃); HPLC: t_{R}=7.17 min (1:1 MeCN:H₂O, 1.0 mL/min, Zorbax™ RX-8).

Example 148b: cyclo(L-2-amino-2-(3′-quinol-4′-onyl)-ethanoyl-L-Ile-D-Pip-L-2-amino-8-hydroxy-decanoyl); TLC: R_{f}=0.18 (1:3:96 NH₄OH:MeOH:CHCl₃); HPLC: t_{R}=5.86 min (1:1 MeCN:H₂O, 1.0 mL/min, Zorbax™ RX-8).

### EXAMPLE 149

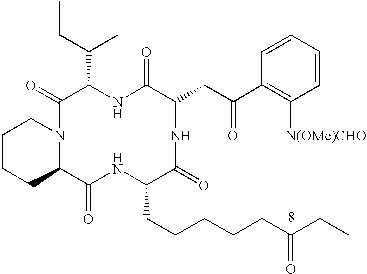

Example 149 was prepared by the following procedure. Ozone was bubbled through 25 mg apicidin in 2.5 mL CH₂Cl₂ at −78° C. until the resulting solution remained pale blue. After 10 min, the solution was purged with a vigorous stream of nitrogen, followed by the addition of 1 mL Me₂S. Then the solution was warmed to RT. The volatiles were removed under reduced pressure and pure Example 149 was obtained following PTLC on silica gel (1×2000 μm plate) using 1:2 acetone:hexanes as eluant. The pure Example 149 thus obtained was characterized by ¹H NMR and MS [m/z: 662.5 (M⁺+Li)].

### EXAMPLE 150

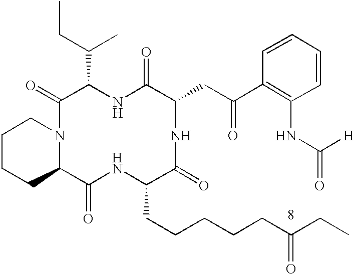

Example 150 was prepared by the following procedure. Ozone was bubbled through a solution of 470 mg N-desmethoxy-apicidin in 40 mL CH₂Cl₂ at −78° C. for about 10 min until a blue color persisted. Then the solution was purged with a vigorous stream on nitrogen, followed by the addition of 1 mL Dimethylsulfide. The resulting solution was allowed to warm to RT and the volatiles were removed under reduced pressure. Following flash chromatography on silica gel using gradient elution (2:3 to 1:1 acetone:hexanes), 320 mg pure Example 150 was obtained which was characterized by ¹H NMR and MS [m/z: 626 (M⁺+1)].

### EXAMPLE 151

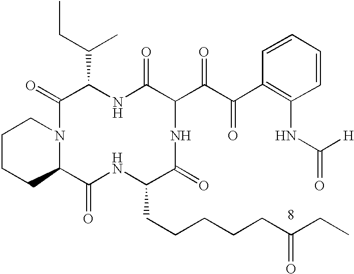

Example 151 was prepared similarly to the procedure described for Example 150 utilizing beta-oxo-N-desmethoxy-apicidin as the starting material. Example 151 thus obtained was characterized by ¹H NMR and MS [m/z: 640 (M⁺+1)].

### EXAMPLES 152A AND 152B

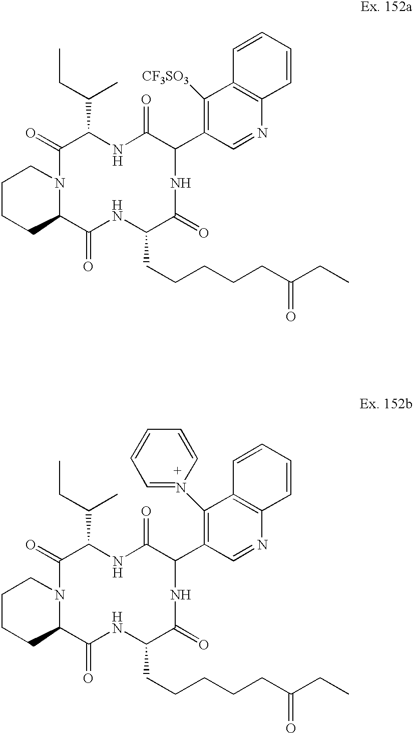

Examples 152a and 152b were prepared by adding 30 μL pyridine to 43 mg cyclo(L-2-amino-2-(3′-quinol-4′-onyl)-ethanoyl-L-Ile-D-Pip-L-2-amino-8-oxo-decanoyl) in 1.2 mL CH₂Cl₂. The mixture was cooled to 0° C. To the resulting solution was added 14 μL (CF₃SO₂)₂O. After 40 min, the solvent was removed in vacuo. The crude pyridinium salt thus obtained was characterized by ¹H NMR and MS [m/z: 740 (M⁺+1)].

### EXAMPLE 153

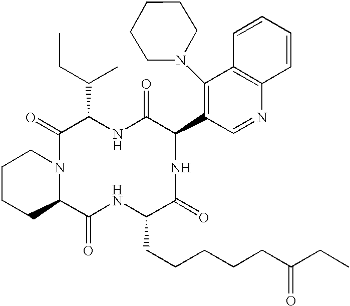

Example 153 was prepared by the following methods L and M.

Method L

To 52 mg crude cyclo(L-2-amino-2-(3′-(4′-pyridium-quinolyl))-ethanoyl-L-Ile-D-Pip-L-2-amino-8-oxo-decanoyl) in 4 mL CH₂Cl₂ at RT was added 1 mg 20% Pd(OH)₂ Degussa catalyst. A hydrogen atmosphere (balloon pressure) was established. After 12 h, the catalyst was removed by filtration through Celite using acetone as eluant. Following PTLC (1×1000 μm plate) on silica gel using 1:3:96 NH₄OH:MeOH:CHCl₃ as eluant, 28 mg pure Example 153 was obtained which was characterized by ¹H NMR and MS [m/z: 675 (M⁺+1)].

Method M

To 20 mg cyclo(L-2-amino-2-(3′-quinol-4′-onyl)-ethanoyl-L-Ile-D-Pip-L-2-amino-8-oxo-decanoyl) in 0.6 mL CH₂Cl₂ at 0° C. was added 8 mg 2,6-di-t-butyl-4-methyl-pyridine followed by 7 μL (CF₃SO₂)₂O. After 3.5 h, 7 μL piperidine was added, the solution was aged for 2.5 h and then was warmed to RT for 12 h. Following PTLC without workup (1×500 μm plate) on silica gel using 1:3:96 NH₄OH:MeOH:CHCl₃ as eluant, 6 mg pure Example 153 was obtained which was characterized by ¹H NMR and MS [m/z: 675 (M⁺+1)].

### EXAMPLE 154

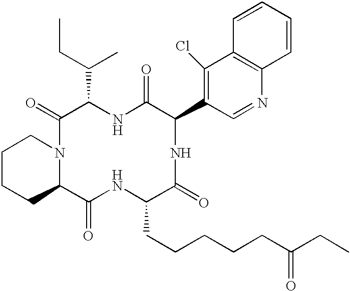

Example 154 was prepared by the following procedure. At 23° C., 13 mg of Example 146a was placed in 360 μL DMF. Then 5.3 mg 2,6-di-tert-butyl-4-methyl-pyridine was added followed by 6.9 mg 2,4-dinitrobenzenesulfonyl chloride. After aging for 6 h, 2.7 mg LiCl was added and the solution was warmed to 60° C. for 12 h. The reaction was cooled to RT, quenched by the addition of water, extracted with CH₂Cl₂ and dried with Na₂SO₄. Following PTLC on silica gel (1×500 μm plate) using 1:9:90 NH₄OH:MeOH:CHCl₃ as eluant, 5 mg pure Example 154 was obtained which was characterized by ¹H NMR and MS [m/z: 626 (M⁺+1)].

### EXAMPLE 155

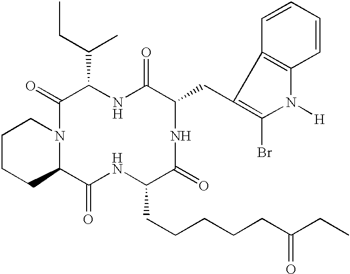

Example 155 was prepared by mixing 1.2 g N-Desmethoxy-apicidin, 360 mg N-bromosuccinamide and 15 mg benzoyl peroxide in 70 mL CCl₄. The resulting mixture was heated to 80° C. for 15 min. The solvent was then removed under reduced pressure and the crude product was purified in two batches by RP-HPLC using 4:6 MeCN:H₂O as eluant to yield 400 mg pure Example 155 which was characterized by ¹H NMR and MS [m/z: 674 (M⁺+1)].

### EXAMPLE 156

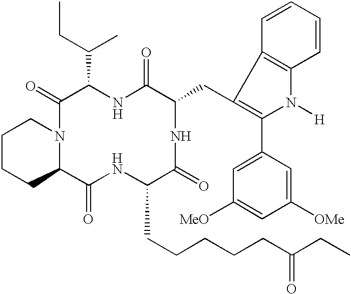

Example 156 was prepared by dissolving 100 mg cyclo(2-bromo-L-Trp-L-Ile-D-Pip-L-2-amino-8-oxo-decanoyl) in 3 mL dioxane and 3 mL EtOH. Then 63 mg LiCl, 270 mg (3,5-diMeO)PhB(OH)₂ and 1.5 mL 1M NaHCO₃ was added. To the resulting mixture was added 17 mg Pd(PPh₃)₄ and the resulting solution was heated in sequence to 90° C. for 90 min, 100° C. for 15 min and 80° C. for 12 h. The solution was poured into 1:1 saturated NaHCO₃:brine, extracted with CH₂Cl₂ and dried with Na₂SO₄. Following preparative TLC on silica gel (1×500 μm plate) using 1:3:96 NH₄OH:MeOH:CHCl₃ as eluant (four developments), 67 pure Example 156 was obtained which was characterized by ¹H NMR and MS [m/z: 730 (M⁺+1)].

### EXAMPLES 157A-157D

Examples 157a-157d were prepared following the procedure described in Example 156.

TABLE 10

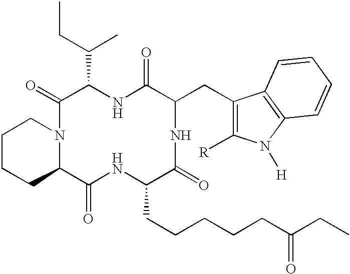

| Example | R Group | Mass Spec |
|---|---|---|
| 156 | Ph(3,5-OMe) | 730 (M⁺ + 1) |
| 157a | 2-napthyl | 720 (M⁺ + 1) |
| 157b | 5-(N-methylindolyl) | 723 (M⁺ + 1) |
| 157c | 1-napthyl | 720 (M⁺ + 1) |
| 157d | Ph | 687 (M⁺ + NH₄) |

### EXAMPLE 158

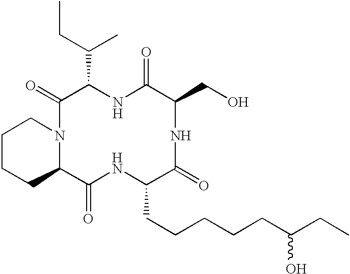

Example 158 was prepared by adding 9 mg NaBH₄ to 100 mg of Example 141 in 6 mL THF at 0° C. After 2 h, the reaction was quenched by the addition of acetone followed by the addition of saturated NaHCO₃(aq), extracted with CH₂Cl₂ and dried with Na₂SO₄. This yielded 10 mg pure diol Example 158 (R_{f}=0.37) following PTLC on silica gel using 3:7 acetone:hexanes as eluant. The product thus obtained was characterized by ¹H NMR.

### EXAMPLE 159

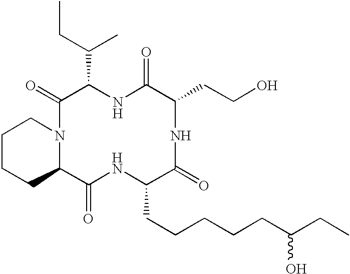

Example 159 was prepared by the following methods N and O.

Method N:

To 100 mg of Example 133 in 3.5 mL THF at 0° C. was added 11.6 mg LiBH₄. After aging for 4 h at 0° C., the reaction was warmed to RT. After an additional 2 h, the reaction was quenched by the addition of acetone followed by the addition of saturated brine(aq), extracted with 3:7 iPrOH:CHCl₃ and dried with Na₂SO₄. Following PTLC on silica gel using 1:3:96 NH₄OH:MeOH:CHCl₃ as eluant, 60 mg pure diol Example 159 was obtained which was characterized by ¹H NMR and MS [m/z: 511 (M⁺+1)].

Method O

To 250 mg of Example 133 in 11 mL THF at 0° C. was added 2.65 mL DIBAL-H (1M solution in toluene). After aging for 4 h at 0° C., the reaction was quenched by the addition of acetone followed by the addition of saturated brine, extracted with 3:7 iPrOH:CHCl₃ and dried with Na₂SO₄. Following flash chromatography on silica gel using 1:3:96 to 1:9:90 NH₄OH:MeOH:CHCl₃ gradient elution, 100 mg pure diol Example 159 (R_{f}=0.41, 1:9:90 NH₄OH:MeOH:CHCl₃) was obtained which was characterized by ¹H NMR.

### EXAMPLE 160

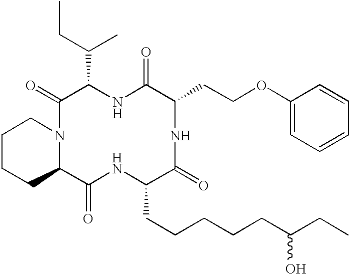

Example 160 was prepared by the following procedure. To 27 mg Ph₃Bi dissolved in 1 mL CH₂Cl₂ at RT was added 0.5 μL CH₃CO₃H. After 10 min, a Ph₃Bi/CH₃CO₃H solution resulted. To the solution, 22 mg of Example 159 was added as a solution in 1 mL CH₂Cl₂, followed by the addition of 3.5 mg Cu(OAc)₂. The resulting solution was then warmed to 60° C. for 3 h. After cooling to RT, the reaction was quenched by the addition of saturated NaHCO₃(aq), extracted with 3:7 iPrOH:CHCl₃ and dried with Na₂SO₄. Following PTLC on silica gel using 4:6 acetone:hexanes (R_{f}=0.66) as eluant, 4 mg pure Example 160 was obtained which was characterized by ¹H NMR and MS [m/z: 587 (M⁺+1)].

### EXAMPLE 161

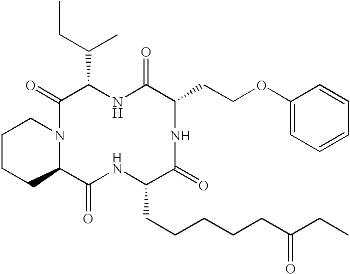

Example 161 was prepared by oxidizing 3 mg of Example 160 using Dess-Martin reagent similarly to the general procedure described in Example 138. This resulted in 2 mg Example 161, which was characterized by ¹H NMR and MS [m/z: 585 (M⁺+1)].

### EXAMPLES 162A AND 162B

Following the general procedure for Examples 160 and 161, the following Examples 162a and 162b were prepared and characterized by NMR and MS:

TABLE 11

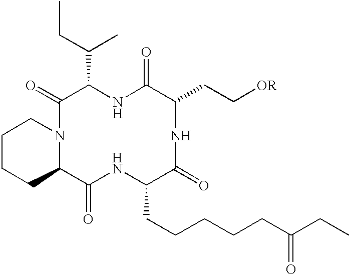

| Example | R Group | Mass Spec |
|---|---|---|
| 161 | Ph | 585 (M⁺ + 1) |
| 162a | Ph(4-OPh) | — |
| 162b | Ph(4-F) | — |

### EXAMPLE 163

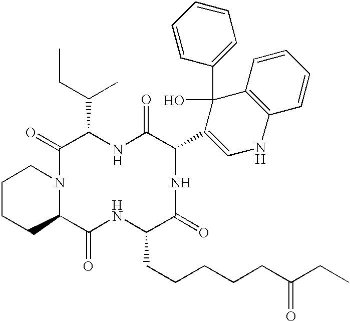

Example 163 was prepared by the following procedure. To 68 mg of Example 148a in 6 mL THF at RT was added 2 mL PhMgBr (2M solution in THF). After aging at RT for 20 h, the reaction was quenched by the addition of saturated NH₄Cl(aq), extracted with CH₂Cl₂ and dried with Na₂SO₄. Following PTLC on silica gel (1×1500 μm plate) using 1:9:90 NH₄OH:MeOH:CHCl₃ as eluant, 18.9 mg pure intermediate compound having a side chain alcohol (R_{f}=0.49, 1:9:90 NH₄OH:MeOH:CHCl₃) was obtained which was characterized by ¹H NMR. The side chain alcohol of the intermediate was then oxidized using Dess-Martin reagent as described in Example 138. Following PTLC on silica gel (1×500 μm plate) using 1:9:90 NH₄OH:MeOH:CHCl₃ as eluant, 13 mg pure Example 163 was obtained (R_{f}=0.66, 1:9:90 NH₄OH:MeOH:CHCl₃) which was characterized by ¹H NMR and MS [m/z: 684 (M⁺+1)].

### EXAMPLES 164A AND 164B

Following the general procedure for Example 163, the following Examples 164a and 164b were prepared:

TABLE 12

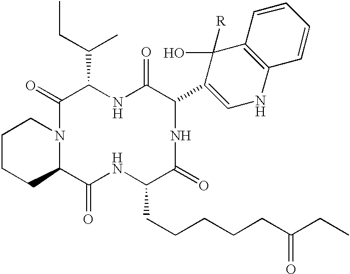

| Example | R Group | Mass Spec |
|---|---|---|
| 163 | Ph | 684 (M⁺ + 1) |
| 164a | Ph(4-tBu) | 757.8 (M⁺ + NH₄) |
| 164b | CH₂Ph | 698 (M⁺ + 1) |

### EXAMPLE 165

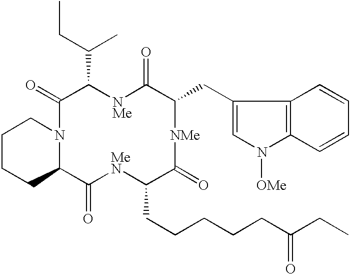

Example 165 was prepared by the following procedure. To 20 mg apicidin in 321 μL DMF at RT was added 16 μL MeI followed by the addition of 3.8 mg NaH (60% suspension in mineral oil). After 20 h, water was added and the solution extracted with EtOAc and dried with Na₂SO₄. Following PTLC on silica gel (1×1000 μm plate) using 1:3:96 NH₄OH:MeOH:CHCl₃ as eluant, 9.9 mg pure Example 165 was obtained which was characterized by ¹H NMR and MS [m/z: 666 (M⁺+1)].

### EXAMPLES 166A-166C

Examples 166a-166c were prepared similarly to the procedure described in Example 165. Apicidin was treated with benzyl bromide, in place of the methyl iodide in Example 165, to yield a mixture of mono-, di- and tri-benzylated derivatives. The three compounds, Examples 166a-166c, thus obtained were characterized by ¹H NMR and MS. The regiochemistry of the mono- and di-benzylated derivatives was not established.

TABLE 13

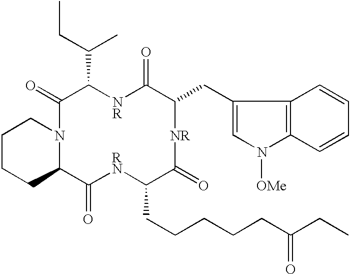

| Example | R Groups | Mass Spec |
|---|---|---|
| 166a | Mono-benzylated | 714 (M⁺ + 1) |
| 166b | Di-benzylated | 804 (M⁺ + 1) |
| 166c | Tri-benzylated | 894 (M⁺ + 1) |

### EXAMPLES 167A-167D

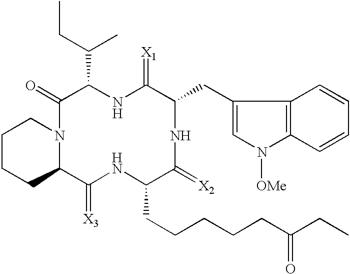

Examples 167a-167d were prepared by the following procedure. To 10 mg apicidin in 2 mL toluene was added 13 mg Lawesson's reagent. The resulting solution was heated at 80° C. for 25 min and then cooled to RT. The entire solution was loaded directly onto a silica gel flash chromatography column and purified by gradient elution (100 % CHCl₃, one column, followed by 1:3:96 NH₄OH:MeOH:CHCl₃ elution) to yield two fractions: monothiono Example 167a (Fraction One—Product A, R_{f}=0.83, 1:3:96 NH₄OH:MeOH:CHCl₃) and impure bis- and tris-thiono Examples 167b-167d (Fraction Two—Products B, C, and D, R_{f}=0.68, 1:3:96 NH₄OH:MeOH:CHCl₃). Fraction Two was further purified by preparative RP-HPLC using gradient elution (2:3 MeCN:H₂O to 100% MeCN, 70 min linear gradient). The products thus obtained were characterized by ¹H NMR and MS. The following retention times were obtained for the four products during the preparative RP-HPLC run:

t_{R}=34.2 min (product A—Example 167a); 39.9 min (product B—Example 167b); 45.6 min (product C—Example 167c); 48.8 min (product D—Example 167d); (2:3 MeCN:H₂O to 100% MeCN, 70 min linear gradient).

TABLE 14

| Example | Product | X₁ | X₂ | X₃ | Mass Spec |
|---|---|---|---|---|---|
| 167a | Product A | S | O | O | 640.3 (M⁺ + 1) |
| 167b | Product B | S | S | O | 656.3 (M⁺ + 1) |
| 167c | Product C | S | O | S | 656.3 (M⁺ + 1) |
| 167d | Product D | S | S | S | 672.3 (M⁺ + 1) |

### EXAMPLE 168

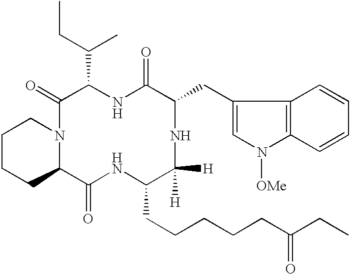

Example 168 was prepared by adding 0.160 mL BH3.THF (1M solution in THF) to 10 mg apicidin in 2 mL THF at 0° C. After 30 min, the resulting solution was warmed to RT and aged for 12 h. At this point, after 12.5 h total, the solution was heated to 60° C. for 30 min, then cooled to RT. Then, 1 mL methanol was added, followed by the addition of 0.15 mL Me₂NCH₂CH₂OH, and the solution was stirred for 2 h. The stirred solution was poured into saturated brine, extracted with EtOAc, and dried with Na₂SO₄. The volatiles were removed under reduced pressure and the crude product was filtered through a 1.5 inch pad of silica gel using 1:3:96 NH₄OH/MeOH/CHCl₃ as eluant to remove baseline contaminants. The filtered solution was concentrated under reduced pressure and pure product was obtained following preparative RP-HPLC using 1/3 MeCN/H₂O isocratic for 20 min, followed by a 60 min linear gradient to 100% MeCN. The pure Example 168 thus obtained was characterized by ¹H NMR and MS [m/z: 612.4 (M⁺+1)]. HPLC: t_{R}=6.69 min, 1/1 MeCN:H₂O, 1.5 ml/min, Zorbax™ RX-8 column. TLC: R_{f}=0.50, 1:3:96 NH₄OH:MeOH:CHCl₃.

### EXAMPLES 169 AND 170

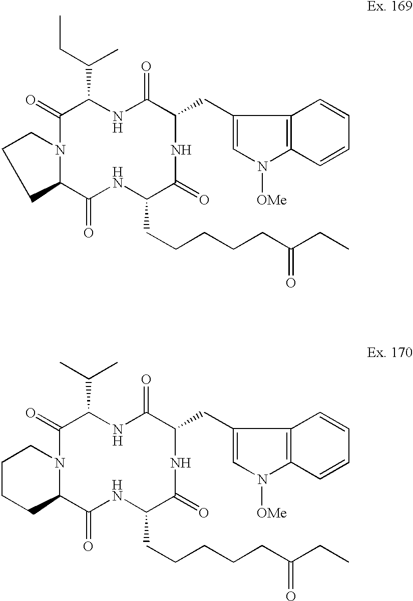

What is claimed is:

1. A compound having a Formula I: 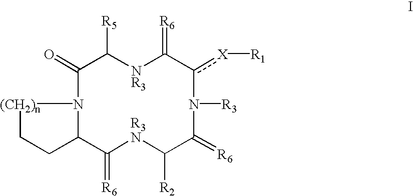
or a pharmaceutically acceptable salt thereof, wherein
X is
(1) —CH₂—,
(2) —C(O)13
(3) —CH(OR^{a})—,
(4) ═CH—, or
(5) not present;
n is
(1) one, or
(2) two;
R₁ is
(1) R₇,
(2) C(O)R₇,
(3) CN,
(4) CO₂R^{b},
(5) C(O)N(OR^{b})R^{c},
(6) C(O)NR^{c}R^{d},
(7) NHCO₂R^{b},
(8) NHC(O)NR^{c}R^{d},
(9) (C₀-C₄alkyl)OR^{a},
(10) (C₀-C₄alkyl)OCO₂R^{b},
(11) (C₀-C₄alkyl)OC(O)NR^{c}R^{d},
(12) C(O)NR^{c}NR^{c}R^{d},
(13) C(O)NR^{c}SO₂R^{b},
(14) OS(O)ₙᵢR₇,
(15) NR^{b}S(O)ₙᵢR₇,
(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a}, C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(17) a benzene ring fused to a 4- to 8-membered heterocyclic ring with from 1 to 4 heteroatoms, optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent;
R₂ is
(1) optionally substituted C₂-C₁₂alkyl,
(2) optionally substituted C₂-C₁₂alkenyl,
(3) optionally substituted C₂-C₁₂alkynyl, or
(4) (CH₂)ₙᵢᵢ—O—(CH₂)ₘᵢᵢ—CH₃,
wherein the optional substituents on the C₂-C₁₂alkyl, C₂-C₁₂alkenyl, and C₂-C₁₂alkynyl are 1 to 8 groups and each group independently is
(a) CO₂R^{a},
(b) C(O)R^{b},
(c) C(O)N(OR^{b})R^{c},
(d) C(O)NR^{c}R^{d},
(e) C(O)NR^{c}NR^{c}R^{d},
(f) C(O)NR^{c}SO₂R₇,
(g) C₃-C₈cycloalkyl,
(h) C₂-C₅alkenyl,
(i) cyano,
(j) ═NOR^{a},
(k) ═NNR^{b}R^{c},
(l) ═NNR^{b}S(O)ₙᵢR₇,
(m) N(OR^{b})C(O)NR^{b}R^{c},
(n) N(OR^{b})C(O)R₇,
(o) NHC(O)N(OR^{b})R^{c},
(p) NR^{c}CO₂R^{b},
(q) NR^{c}C(O)NR^{c}R^{d},
(r) NR^{c}C(S)NR^{c}R^{d},
(s) NR^{c}C(O)R₇,
(t) NR^{b}S(O)ₙᵢR₇,
(u) NR^{c}CH₂CO₂R^{a},
(v) NR^{c}C(S)R₇,
(x) NR^{c}C(O)CH₂OH,
(y) NR^{c}C(O)CH₂SH,
(z) NR^{c}CH₂CH(OH)R₇,
(aa) NR^{c}P(O)(OR^{a})R₇,
(bb) NY¹Y², wherein Y¹ and Y² are independently H or C₁-C₁₀alkyl,
(cc) NO₂,
(dd) N(OR^{b})C(O)R^{b},
(ee) C₁-C₁₀alkanoylamino,
(ff) OR^{a},
(gg) OS(O)ₙᵢR₇,
(hh) oxo,
(ii) OCO₂R^{b},
(jj) OC(O)NR^{c}R^{d},
(kk) P(O)(OR^{a})₂,
(ll) P(O)(OR^{a})R₇,
(mm) SC(O)R₇,
(nn) S(O)ₙᵢR₇,
(oo) SR₇,
(pp) S(O)ₙᵢNR^{c}R^{d},
(qq) diazo,
(rr) C₁-C₅ perfluoroalkyl,
(ss) B(O)(OR^{a})OR^{a},
(tt) halogen,
(uu) aryl(C₀-C₅alkyl), wherein the aryl is optionally substituted with 1 to 3 groups, wherein each group independently is R^{f}, or
(vv) a 3- to 8-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle is saturated or partly unsaturated;
R₃ each independently is
(1) hydrogen,
(2) halogen,
(3) OR^{a},
(4) C₁-C₄alkyl, or
(5) aryl;
R₅ is
(1) isopropyl, or
(2) sec-butyl;
R₆ each independently is
(1) O,
(2) S, or
(3) H;
R₇ is
(1) hydrogen,
(2) optionally substituted C₂-C₁₀alkyl,
(3) optionally substituted C₂-C₁₀alkenyl,
(4) optionally substituted C₂-C₁₀alkynyl,
(5) optionally substituted C₃-C₈cycloalkyl,
(6) optionally substituted C₅-C₈cycloalkenyl,
(7) optionally substituted aryl,
wherein the optional substituents on the C₂-C₁₀alkyl, C₂-C₁₀alkenyl, C₂-C₁₀alkynyl, C₃-C₈cycloalkyl, C₅-C₈cycloalkenyl and aryl are 1 to 4 groups, and each group independently is
(a) C₁-C₅alkyl,
(b) X¹-C₁-C₁₀alkyl, wherein X¹ is O or S(O)ₙᵢ,
(c) C₃-C₈cycloalkyl,
(d) hydroxy,
(e) halogen,
(f) cyano,
(g) carboxy,
(h) NY¹Y², wherein Y¹ and Y² are independently H or C₁-C₁₀alkyl,
(i) nitro,
(j) C₁-C₁₀alkanoylamino,
(k) aroyl amino wherein the aroyl is optionally substituted with 1 to 3 groups wherein each group independently is R^{f1}, wherein R^{f1} is defined by any of the definitions below for R^{f} except for (14), (26), (27), and (32),
(l) oxo,
(m) aryl C₀-C₅alkyl wherein the aryl is optionally substituted with 1 to 3 groups, wherein each group independently is R^{f1},
(q) C₁-C₅perfluoroalkyl,
(r) N(OR^{b})C(O)R_{7′}, wherein R₇′ is any of the above definitions of R₇ from (1) to (7)(m), and below of R₇ from (8) to (12), or
(s) NR^{c}C(O)R_{7′},
(8) a 5- to 10-membered heterocycle containing from 1 to 4 heteroatoms, each heteroatom independently is oxygen, sulfur or nitrogen and the heterocycle is optionally substituted by 1 to 3 groups, each group independently is R^{f1}, and the heterocycle is saturated or partly unsaturated,
(9) a benzene ring fused to a 5- to 10-membered heterocyclic ring containing from 1 to 4 heteroatoms, each heteroatom independently is oxygen, sulfur or nitrogen and the heterocycle is optionally substituted by 1 to 3 groups, each group independently is R^{f1}, and the heterocycle is saturated or partly unsaturated,
(10) a 5- to 10-membered heterocyclic ring containing from 1 to 4 heteroatoms fused to a second 5- to 10-membered heterocyclic ring containing from 1 to 4 heteroatoms, each heteroatom in either heterocyclic ring independently is oxygen, sulfur or nitrogen and the second heterocyclic ring is optionally substituted by 1 to 3 groups, each group independently is R^{f1}, and each heterocycle independently is saturated or partly unsaturated,
(11) a benzene ring fused to a C₃-C₈cycloalkyl ring, wherein the cycloalkyl is optionally substituted by 1 to 3 groups each independently being R^{f1}, and the cycloalkyl ring is saturated or partly unsaturated, or
(12) a 5- to 10-membered heterocyclic ring containing from 1 to 4 heteroatoms, each heteroatom independently is oxygen, sulfur or nitrogen, the heterocyclic ring is fused to a C₃-C₈cycloalkyl ring, wherein the cycloalkyl ring is optionally substituted by 1 to 3 groups each independently being R^{f1}, and the cycloalkyl ring is saturated or partly unsaturated,
R^{a} is
(1) hydrogen,
(2) optionally substituted C₁-C₁₀alkyl,
(3) optionally substituted C₃-C₁₀alkenyl,
(4) optionally substituted C₃-C₁₀alkynyl,
(5) optionally substituted C₁-C₁₀alkanoyl,
(6) optionally substituted C₃-C₁₀alkenoyl,
(7) optionally substituted C₃-C₁₀alkynoyl,
(8) optionally substituted aroyl,
(9) optionally substituted aryl,
(10) optionally substituted C₃-C₇cycloalkanoyl,
(10) optionally substituted C₅-C₇cycloalkenoyl,
(12) optionally substituted C₁-C₁₀alkylsulfonyl,
(13) optionally substituted C₃-C₈cycloalkyl,
(14) optionally substituted C₅-C₈cycloalkenyl,
wherein the optional substituents on the C₁-C₁₀alkyl, C₃-C₁₀alkenyl, C₃-C₁₀alkynyl, C₁-C₁₀alkanoyl, C₃-C₁₀alkenoyl, C₃-C₁₀alkynoyl, aroyl, aryl, C₃-C₇cycloalkanoyl, C₅-C₇cycloalkenoyl, C₁-C₁₀alkylsulfonyl, C₃-C₈cycloalkyl and C₅-C₈cycloalkenyl are from 1 to 10 groups, wherein each group independently is hydroxy, C₁-C₆alkoxy, C₃-C₇cycloalkyl, aryl C₁-C₃alkoxy, NR^{x}R^{x}, CO₂R^{b}, CONR^{c}R^{d}, or halogen,
(15) C₁-C₅perfluoroalkyl,
(16) arylsulfonyl optionally substituted with 1 to 3 groups, wherein each group independently is C₁-C₅alkyl, C₁-C₅perfluoroalkyl, nitro, halogen or cyano,
(17) a 5- or 6-membered heterocycle containing 1 to 4 heteroatoms, wherein each heteroatom is oxygen, sulfur or nitrogen, wherein the heterocycle is optionally substituted by 1 to 4 groups, wherein each group independently is C₁-C₅alkyl, C₂-C₅alkenyl, C₁-C₅perfluoroalkyl, amino, NMe₂, C(O)NR^{c}R^{d}, cyano, CO₂R^{b} or halogen, and wherein the heterocycle is saturated or partly unsaturated, or
(18) OP(O)(OR^{b})₂;
R^{b} is
(1) H,
(2) optionally substituted aryl,
(3) optionally substituted C₁-C₁₀alkyl,
(4) optionally substituted C₃-C₁₀alkenyl,
(5) optionally substituted C₃-C₁₀alkynyl,
(6) optionally substituted C₃-C₁₅cycloalkyl,
(7) optionally substituted C₅-C₁₀cycloalkenyl, or
(8) optionally substituted 5- to 10-membered heterocycle containing 1 to 4 heteroatoms, wherein each heteroatom independently is oxygen, sulfur, or nitrogen,
wherein the optional substituents on the aryl, C₁-C₁₀alkyl, C₃-C₁₀ alkenyl, C₃-C₁₀alkynyl, C₃-C₁₅cycloalkyl, C₅-C₁₀cycloalkenyl, or 5- to 10-membered heterocycle are from 1 to 10 groups, wherein each group independently is
(a) hydroxy,
(b) C₁-C₆alkyl,
(c) oxo,
(d) SO₂NR^{x}R^{x},
(e) aryl C₁-C₆alkoxy,
(f) hydroxy C₁-C₆alkyl,
(g) C₁-C₁₂alkoxy,
(h) hydroxy C₁-C₆alkoxy,
(i) amino C₁-C₆alkoxy,
(j) cyano,
(k) mercapto,
(l) (C₁-C₆alkyl)—S(O)ₙᵢ—(C₀-C₆alkyl),
(m) C₃-C₇cycloalkyl optionally substituted with 1 to 4 groups, wherein each group independently is R^{e},
(n) C₅-C₇cycloalkenyl,
(o) halogen,
(p) C₁-C₅alkanoyloxy,
(q) C(O)NR^{x}R^{x},
(r) CO₂Rⁱ,
(s) formyl,
(t) —NR^{x}R^{x},
(u) 5 to 9-membered heterocycle, which is saturated or partially unsaturated, containing from 1 to 4 heteroatoms, wherein each heteroatom independently is oxygen, sulfur or nitrogen, and the heterocycle is optionally substituted with 1 to 5 groups, wherein each group independently is R^{e},
(v) optionally substituted aryl, wherein the optional substituents are 1,2-methylenedioxy or 1 to 5 groups, wherein each group independently is R^{e},
(w) optionally substituted aryl C₁-C₃alkoxy, wherein the optional substituents are 1,2-methylenedioxy or 1 to 5 groups, wherein each group independently is R^{e}, or
(x) C₁-C₅perfluoroalkyl;
R^{c} and R^{d} are independently selected from R^{b}; or R^{c} and R^{d} together with the N to which they are attached form a 3- to 10-membered ring containing 0 to 2 additional heteroatoms, each additional heteroatom independently being oxygen, nitrogen, or (O)ₙᵢ substituted sulfur, wherein the ring is optionally substituted with 1 to 3 groups, wherein each group independently is R^{g}, hydroxy, thioxo, or oxo;
R^{e} is
(1) halogen,
(2) C₁-C₇alkyl,
(3) C₁-C₃perfluoroalkyl,
(4) —S(O)ₘRⁱ,
(5) cyano,
(6) nitro,
(7) RⁱO(CH₂)ᵥ—,
(8) RⁱCO₂(CH₂)ᵥ—,
(9) RⁱOCO(CH₂)ᵥ,
(10) optionally substituted aryl wherein the optional substituents are from 1 to 3 groups, wherein each group independently is halogen, C₁-C₆alkyl, C₁-C₆alkoxy, or hydroxy,
(11) SO₂NR^{x}R^{x},
(12) CO₂R^{x}, or
(13) NR^{x}R^{x};
R^{f} is
(1) C₁-C₄alkyl,
(2) X¹-C₁-C₄alkyl, wherein X¹ is O or S(O)ₘ,
(3) C₂-C₄alkenyl,
(4) C₂-C₄ alkynyl,
(5) C₁-C₃perfluoroalkyl,
(6) NY³Y⁴, wherein Y³ and Y⁴ are each independently hydrogen, C₁-C₅alkyl, or SO₂R^{b},
(7) hydroxy,
(8) halogen,
(9) C₁-C₅alkanoyl amino,
(10) (C₀-C₄alkyl)CO₂R^{a},
(11) (C₀-C₄alkyl)C(O)NR^{b}R^{c},
(12) (C₀-C₄alkyl)NY⁵Y⁶ wherein Y⁵ and Y⁶ together with the N to which they are attached form a 3- to 7-membered ring containing 0 to 2 additional heteroatoms, wherein the additional heteroatoms independently are oxygen, nitrogen, or (O)ₘᵢ substituted sulfur, wherein the ring is optionally substituted with 1 to 3 groups, wherein each group independently is R^{e} or oxo,
(13) (C₀-C₄alkyl)NO₂,
(14) (C₀-C₄alkyl)C(O)R₇,
(15) (C₀-C₄alkyl)CN,
(16) oxo,
(17) (C₀-C₄alkyl)C(O)N(OR^{b})R^{c},
(18) (C₀-C₄alkyl)C(O)NR^{c}R^{d},
(19) (C₀-C₄alkyl)NHC(O)OR^{b},
(20) (C₀-C₄alkyl)NHC(O)NR^{c}R^{d},
(21) (C₀-C₄alkyl)OR^{a},
(22) (C₀-C₄alkyl)OCO₂R^{b},
(23) (C₀-C₄alkyl)OC(O)NR^{c}R^{d},
(24) (C₀-C₄alkyl)C(O)NR^{c}NR^{c}R^{d},
(25) (C₀-C₄alkyl)C(O)NR^{c}SO₂R^{b},
(26) (C₀-C₄alkyl)OS(O)ₙᵢR₇,
(27) (C₀-C₄alkyl)NR^{b}S(O)ₙᵢR₇,
(28) C₀-C₄alkyl halogen,
(29) (C₀-C₄alkyl) SR^{a},
(30) P(O)(OR^{a})₂,
(31) C₀-C₄alkyl azide,
(32) aryl substituted with from 1 to 4 groups, wherein each group independently is S(O)₂R₇, CO₂R^{b}, C(O)NR^{c}R^{d}, NO₂, halogen, OC(O)R^{a}, OR^{a} or C₁-C₄alkyl;
R^{g} is
(1) hydrogen,
(2) C₁-C₆alkyl optionally substituted with hydroxy, amino, or CO₂Rⁱ,
(3) aryl optionally substituted with halogen, 1,2-methylenedioxy, C₁-C₇alkoxy, C₁-C₇alkyl, or C₁-C₃perfluoroalkyl,
(4) aryl C₁-C₆alkyl, wherein the aryl is optionally substituted with C₁-C₃perfluoroalkyl or 1,2-methylenedioxy,
(5) C₁-C₅alkoxycarbonyl,
(6) C₁-C₅alkanoyl,
(7) C₁-C₅alkanoyl C₁-C₆alkyl,
(8) arylC₁-C₅ alkoxycarbonyl,
(9) aminocarbonyl,
(10) (C₁-C₅monoalkyl)aminocarbonyl,
(11) (C₁-C₅dialkyl)aminocarbonyl, or
(12) CO₂R^{b};
Rⁱ is
(1) hydrogen,
(2) C₁-C₃perfluoroalkyl,
(3) C₁-C₆alkyl, or
(4) optionally substituted aryl C₀-C₆alkyl, wherein the aryl optional substituents are from 1 to 3 groups, wherein each group independently is halogen, C₁-C₆alkyl, C₁-C₆alkoxy, or hydroxy;
R^{x} is a C₁-C₄alkyl;
m is 0 to 2;
mi is 0 to 2;
ni is 0 to 2;
mii is 0 to 6;
nii is 0 to 7;
v is 0 to 3; and
excluding apicidin, N-desmethoxy apicidin, chlamydocin, Cly-2, HC-Toxin, Trapoxin A, β-hydroxy-HC-toxin and compounds represented by chemical Formula IIA and chemical Formula IIB: 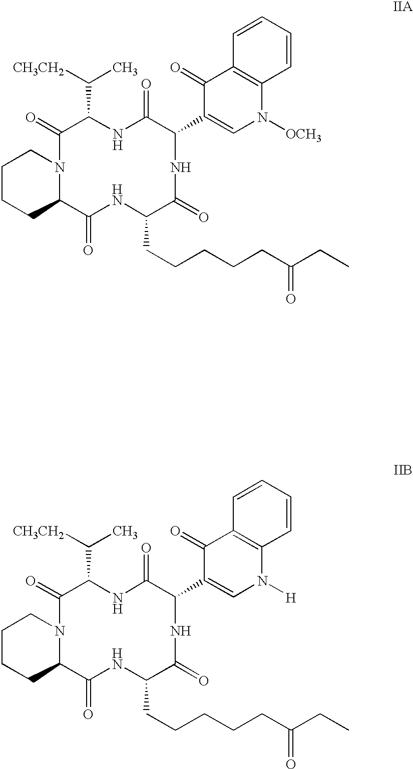
and excluding compounds having the formula IIC 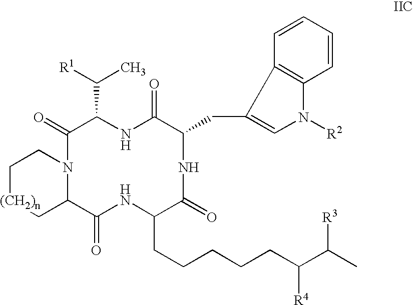
wherein R¹ is CH₃ or CH₂CH₃;
R² is H or —OCH₃;
R³ is H and R⁴ is ═O or (H, OH); or
R³ is OH and R⁴ is ═O or (H, OH); and
n is 0 or 1.

2. The compound according to claim 1, or a pharmaceutically acceptable salt thereof, wherein:
X is
(1) —CH₂—,
(2) —C(O)—,
(3) —CH(OR^{a})—, or
(4) not present.

3. The compound according to claim 1, or a pharmaceutically acceptable salt thereof, wherein:
X is
—CH(OR^{a})—.

4. The compound according to claim 3, or a pharmaceutically acceptable salt thereof, wherein n is 1.

5. The compound according to claim 2, or a pharmaceutically acceptable salt thereof, wherein:
X is
(1) —CH₂—,
(2) —C(O)—, or
(3) not present.

6. The compound according to claim 5, or a pharmaceutically acceptable salt thereof, wherein n is 1.

7. The compound according to claim 2, or a pharmaceutically acceptable salt thereof, wherein:
X is
(1) —CH₂—,
(2) —C(O)—, or
(3) not present; and
R₁ is
(1) R₇,
(2) C(O)R₇,
(15) CO₂R^{b},
(16) C(O)N(OR^{b})R^{c},
(17) C(O)NR^{c}R^{d},
(18) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(19) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(20) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

8. The compound according to claim 7, or a pharmaceutically acceptable salt thereof, wherein n is 1.

9. The compound according to claim 2, or a pharmaceutically acceptable salt thereof, wherein:
X is
(1) —CH₂—,
(2) —C(O)—, or
(3) not present;
RI is
(1) R₇,
(9) C(O)R₇,
(10) CO₂R^{b},
(11) C(O)N(OR^{b})R^{c},
(12) C(O)NR^{c}R^{d},
(13) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(14) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(15) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent; and
R₂ is
(1) optionally substituted C₂-C₁₂alkyl,
(2) optionally substituted C₂-C₁₂alkenyl,
(3) optionally substituted C₂-C₁₂alkynyl, or
(4) (CH₂)ₙᵢᵢ—O—(CH₂)ₘᵢᵢ—CH₃, wherein the optional substituents on the C₂-C₁₂alkyl, C₂-C₁₂alkenyl, and C₂-C₁₂alkynyl are 1 to 5 groups and each group independently is
(a) CO₂R^{a},
(b) C(O)R^{b},
(c) C(O)N(OR^{b})R^{c},
(d) C(O)NR^{c}R^{d},
(e) C(O)NR^{c}NR^{c}R^{d},
(f) C(O)NR^{c}SO₂R₇,
(g) C₃-C₈cycloalkyl,
(h) C₂-C₅alkenyl,
(i) cyano,
(j) ═NOR^{a},
(k) ═NNR^{b}R^{c},
(l) ═NNR^{b}S(O)ₙᵢR₇,
(m) N(OR^{b})C(O)NR^{b}R^{c},
(n) N(OR^{b})C(O)R₇,
(o) NHC(O)N(OR^{b})R^{c},
(p) NR^{c}CO₂R^{b},
(q) NR^{c}C(O)NR^{c}R^{d},
(r) NR^{c}C(S)NR^{c}R^{d},
(s) NR^{c}C(O)R₇,
(t) NR^{b}S(O)ₙᵢR₇,
(u) NR^{c}CH₂CO₂R^{a},
(v) NR^{c}C(S)R₇,
(x) NR^{c}C(O)CH₂OH,
(y) NR^{c}C(O)CH₂SH,
(z) NR^{c}CH₂CH(OH)R₇,
(aa) NR^{c}P(O)(OR^{a})R₇,
(bb) NY¹Y², wherein Y¹ and Y² are independently H or methyl,
(cc) NO₂,
(dd) N(OR^{b})C(O)R^{b},
(ee) C₁-C₃alkanoylamino,
(ff) OR^{a},
(gg) OS(O)ₙᵢR₇,
(hh) oxo,
(ii) OCO₂R^{b},
(jj) OC(O)NR^{c}R^{d},
(kk) P(O)(OR^{a})₂,
(l) P(O)(OR^{a})R₇,
(mm) SC(O)R₇,
(nn) S(O)ₙᵢR₇,
(oo) SR₇,
(pp) S(O)ₙᵢNR^{c}R^{d},
(qq) diazo,
(rr) C₁-C₅ perfluoroalkyl,
(ss) B(O)(OR^{a})OR^{a},
(tt) halogen,
(uu) aryl(C₀-C₅alkyl), wherein the aryl is optionally substituted with 1 to 3 groups, wherein each group independently is R^{f}, or
(xxii) a 3- to 6-membered heterocycle containing from 1 to 4 heteroatoms, each heteroatom independently is oxygen, sulfur or nitrogen, wherein the heterocycle is optionally substituted by 1 to 3 groups, wherein each group independently is R^{f}, and the heterocycle is saturated or partly unsaturated.

10. The compound according to claim 9, or a pharmaceutically acceptable salt thereof, wherein n is 1.

11. The compound according to claim 1, or a pharmaceutically acceptable salt thereof, wherein:
R₃ each independently is
(1) hydrogen,
(2) halogen,
(3) OR^{a},
(4) C₁-C₄alkyl, or
(5) aryl; and
R^{a} is
(1) hydrogen,
(2) optionally substituted C₁-C₆alkyl,
(3) optionally substituted C₃-C₆alkenyl,
(4) optionally substituted C₂-C₄alkanoyl,
(5) optionally substituted C₃-C₄alkenoyl,
(6) optionally substituted aroyl,
(7) optionally substituted aryl,
(8) optionally substituted C₅-C₆cycloalkanoyl,
(9) optionally substituted C₁-C₄alkylsulfonyl,
(10) optionally substituted C₅-C₆cycloalkyl,
(11) optionally substituted C₅-C₆cycloalkenyl, wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,
(12) CF₃,
(13) arylsulfonyl optionally substituted with 1 to 3 groups, wherein each group independently is methyl, CF₃, nitro, halogen or cyano, or
(14) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle is saturated or partly unsaturated.

12. The compound according to claim 11, or a pharmaceutically acceptable salt thereof, wherein:
R₃ each independently is
(1) hydrogen,
(2) halogen,
(3) OR^{a},
(4) C₁-C₄alkyl, or
(5) aryl;
R^{a} is
(1) hydrogen,
(2) optionally substituted C₁-C₆alkyl,
(3) optionally substituted C₃-C₆alkenyl,
(5) optionally substituted C₂-C₄alkanoyl,
(6) optionally substituted C₃-C₄alkenoyl,
(8) optionally substituted aroyl,
(9) optionally substituted aryl,
(10) optionally substituted C₅-C₆cycloalkanoyl,
(12) optionally substituted C₁-C₄alkylsulfonyl,
(13) optionally substituted C₅-C₆cycloalkyl,
(14) optionally substituted C₅-C₆cycloalkenyl,
wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,
(15) CF₃,
(16) arylsulfonyl optionally substituted with 1 to 3 groups, wherein each group independently is methyl, CF₃, nitro, halogen or cyano, or
(17) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle is saturated or partly unsaturated;
X is
(1) —CH₂—,
(2) —C(O)—,
(3) ═CH—, or
(4) not present; and
R₁ is
(1) R₇,
(2) C(O)R₇,
(3) CN,
(4) CO₂R^{b},
(5) C(O)N(OR^{b})R^{c},
(6) C(O)NR^{c}R^{d},
(7) NHCO₂R^{b},
(8) NHC(O)NR^{c}R^{d},
(9) (C₀-C₄alkyl)OR^{a},
(10) (C₀-C₄alkyl)OCO₂R^{b},
(11) (C₀-C₄alkyl)OC(O)NR^{c}R^{d},
(12) C(O)NR^{c}NR^{c}R^{d},
(13) C(O)NR^{c}SO₂R^{b},
(14) OS(O)ₙᵢR₇,
(15) NR^{b}S(O)ₙᵢR₇,
(16) a 3- to 8-membered heterocycle containing 1 to 4 heteroatoms, optionally substituted by 1 to 4 groups, each group independently is C₁-C₆alkyl, C₂-C₅alkenyl, C₁-C₅perfluoroalkyl, NR^{c}R^{d}, oxo, thiono, OR^{a}, S(O)ₙᵢR^{a}, C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

13. The compound according to 12, or a pharmaceutically acceptable salt thereof, wherein n is 1.

14. The compound according to claim 11, or a pharmaceutically acceptable salt thereof, wherein:
R₃ each independently is
(1) hydrogen,
(2) halogen,
(3) OR^{a},
(4) C₁-C₄alkyl, or
(5) aryl;
R^{a} is
(1) hydrogen,
(2) optionally substituted C₁-C₆alkyl,
(3) optionally substituted C₃-C₆alkenyl,
(5) optionally substituted C₂-C₄alkanoyl,
(6) optionally substituted C₃-C₄alkenoyl,
(8) optionally substituted aroyl,
(9) optionally substituted aryl,
(10) optionally substituted C₅-C₆cycloalkanoyl,
(12) optionally substituted C₁-C₄alkylsulfonyl,
(13) optionally substituted C₅-C₆cycloalkyl,
(14) optionally substituted C₅-C₆cycloalkenyl,
wherein the optional substituents on the C₁-C₆alkyl, C₃-C₆alkenyl, C₂-C₄alkanoyl, C₃-C₄alkenoyl, aroyl, aryl, C₅-C₆cycloalkanoyl, C₁-C₄alkylsulfonyl, C₅-C₆cycloalkyl and C₅-C₆cycloalkenyl are from 1 to 10 groups, wherein each group independently is hydroxy, methoxy, aryl methoxy, NR^{x}R^{x}, CO₂R^{b}, CONR^{c}R^{d}, or halogen,
(15) CF₃,
(16) arylsulfonyl optionally substituted with 1 to 3 groups, wherein each group independently is methyl, CF₃, nitro, halogen or cyano, or
(17) a 5- or 6-membered heterocycle containing 1 to 3 heteroatoms, wherein each heteroatom is oxygen, sulfur or nitrogen, wherein the heterocycle is optionally substituted by 1 to 3 groups, wherein each group independently is methyl, CF₃, NMe₂, C(O)NR^{c}R^{d}, cyano, CO₂R^{b} or halogen, and wherein the heterocycle is saturated or partly unsaturated;
X is
(1) —CH₂—,
(2) —C(O)—,
(3) ═CH—, or
(4) not present; and
R₁ is
(1) R₇,
(2) C(O)R₇,
(4) CO₂R^{b},
(5) C(O)N(OR^{b})R^{c},
(6) C(O)NR^{c}R^{d},
(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a}, C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

15. The compound according to claim 14, or a pharmaceutically acceptable salt thereof, wherein n is 1.

16. The compound according to claim 1, or a pharmaceutically acceptable salt thereof, wherein:
R₆ each independently is
(1) O,
(2) S, or
(3) H;
X is
(1) —CH₂—,
(2) —C(O)—,
(3) ═CH—, or
(4) not present; and
R₁ is
(1) R₇,
(2) C(O)R₇,
(3) CN,
(4) CO₂R^{b},
(5) C(O)N(OR^{b})R^{c},
(6) C(O)NR^{c}R^{d},
(7) NHCO₂R^{b},
(8) NHC(O)NR^{c}R^{d},
(9) (C₀-C₄alkyl)OR^{a},
(10) (C₀-C₄alkyl)OCO₂R^{b},
(11) (C₀-C₄alkyl)OC(O)NR^{c}R^{d},
(12) C(O)NR^{c}NR^{c}R^{d},
(13) C(O)NR^{c}SO₂R^{b},
(14) OS(O)ₙᵢR₇,
(15) NR^{b}S(O)ₙᵢR₇,
(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a}, C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

17. The compound according to claim 16, or a pharmaceutically acceptable salt thereof, wherein n is 1.

18. The compound according to claim 16, or a pharmaceutically acceptable salt thereof wherein:
R₃ each independently is
(1) hydrogen,
(2) halogen,
(3) OR^{a},
(4) C₁-C₄alkyl, or
(5) C₁-C₄aryl;
R₆ each independently is
(1) O,
(2) S, or
(3) H;
X is
(1) —CH₂—,
(2) —C(O)—,
(3) ═CH—, or
(4) not present; and
R₁ is
(1) R₇,
(2) C(O)R₇,
(3) CN,
(4) CO₂R^{b},
(5) C(O)N(OR^{b})R^{c},
(6) C(O)NR^{c}R^{d},
(7) NHCO₂R^{b},
(8) NHC(O)NR^{c}R^{d},
(9) (C₀-C₄alkyl)OR^{a},
(10) (C₀-C₄alkyl)OCO₂R^{b},
(11) (C₀-C₄alkyl)OC(O)NR^{c}R^{d},
(12) C(O)NR^{c}NR^{c}R^{d},
(13) C(O)NR^{c}SO₂R^{b},
(14) OS(O)ₙᵢR₇,
(15) NR^{b}S(O)ₙᵢR₇, wherein ni is from 0 to 2,
(16) a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent,
(17) a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide, or
(18) a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

19. The compound according to claim 18, or a pharmaceutically acceptable salt thereof, wherein n is 1 or 2.

20. The compound according to claim 1, or a pharmaceutically acceptable salt thereof, wherein X is —CH₂—.

21. The compound according to claim 1, or a pharmaceutically acceptable salt thereof, wherein X is —C(O)—.

22. The compound according to claim 1, or a pharmaceutically acceptable salt thereof, wherein X is not present.

23. The compound according to claim 1, or a pharmaceutically acceptable salt thereof, wherein R₁ is a 3- to 8-membered heterocycle containing 1 to 4 heteroatoms, optionally substituted by 1 to 4 groups, each group independently is C₁-C₅alkyl, C₂-C₅alkenyl, C₁-C₅perfluoroalkyl, NR^{c}R^{d}, oxo, thiono, OR^{a}, S(O)ₙᵢR^{a} (where ni=0, 1 or 2), C(O)R^{a}, C(O)NR^{c}R^{d}, cyano, (C₀-C₆alkyl)aryl, CO₂R^{b}, or halogen, and each group is saturated, partly unsaturated or fully unsaturated, wherein the heteroatoms are each independently oxygen, sulfur, or nitrogen, in which the nitrogen optionally has an R^{c} substituent.

24. The compound according to claim 1, or a pharmaceutically acceptable salt thereof, wherein RI is a benzene ring fused to a 4- to 8-membered heterocyclic ring with from 1 to 4 heteroatoms, optionally substituted by 1 to 4 groups each independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, each group is saturated, partly unsaturated, or fully unsaturated, wherein the heteroatoms are each independently oxygen, sulfur, or nitrogen, in which the nitrogen optionally has an R^{c} substituent, and wherein the benzene/heterocycle fused ring is attached at any site to X or to the tetrapeptide.

25. The compound according to claim 1, or a pharmaceutically acceptable salt thereof, wherein R₁ is a 4- to 8-membered heterocyclic ring with from 1 to 4 heteroatoms fused to a second 4- to 8-membered heterocyclic ring with from 1 to 4 heteroatoms, each heterocyclic ring independently optionally substituted by 1 to 4 groups, each group independently is C₁-C₅alkyl, C₂-C₅alkenyl, C₁-C₅perfluoroalkyl, amino, oxo, thiono, C(O)NR^{c}R^{d}, cyano, CO₂R^{b} or halogen, wherein each heterocycle is saturated, partly unsaturated or fully unsaturated, and wherein each heteroatom independently is oxygen, sulfur, or nitrogen, and the nitrogen optionally has an R^{c} substituent.

26. A pharmaceutical composition comprising a therapeutically effective amount of a compound according to claim 1 and a pharmaceutically acceptable carrier.

27. A method for the treatment of protozoal infections comprising the step of administering, to a host in need of such treatment, a non-toxic amount of a compound according to claim 1, or a salt thereof, effective to inhibit a histone deacetylase activity of the infecting protozoa.

28. A method for the prevention of protozoal infections comprising the step of administering to a host a non-toxic effective preventative amount of a compound according to claim 1, or a salt thereof.

29. The compound according to claim 3, or a pharmaceutically acceptable salt thereof, wherein n is 2.

30. The compound according to claim 5, or a pharmaceutically acceptable salt thereof, wherein n is 2.

31. The compound according to claim 7, or a pharmaceutically acceptable salt thereof, wherein n is 2.

32. The compound according to claim 9, or a pharmaceutically acceptable salt thereof, wherein n is 2.

33. The compound according to 12, or a pharmaceutically acceptable salt thereof, wherein n is 2.

34. The compound according to claim 14, or a pharmaceutically acceptable salt thereof, wherein n is 2.

35. The compound according to claim 16, or a pharmaceutically acceptable salt thereof, wherein n is 2.
